(19) United States
(12) Patent Application Publication
(10) Pub. No.: US 2023/0286983 A1
(43) Pub. Date: Sep. 14, 2023

(54) PYRIDINE-1,5-DIONES EXHIBITING MNK INHIBITION AND THEIR METHOD OF USE

(71) Applicant: 4E Therapeutics, Inc., Austin, TX (US)

(72) Inventors: Theodore J. Price, Austin, TX (US); James J. Sahn, Austin, TX (US)

(21) Appl. No.: 18/003,829

(22) PCT Filed: Jun. 30, 2021
(86) PCT No.: PCT/US2021/039982

§ 371 Date: Dec. 29, 2022

Related U.S. Application Data
(60) Provisional application No. 63/046,325, filed on Jun. 30, 2020.

(51) Int. Cl. C07D 471/14; C07D 491/147; A61P 25/00

(52) U.S. Cl. CPC C07D 471/14; C07D 491/147; A61P 25/00

(57) ABSTRACT

Compounds having activity as inhibitors of MNK are provided. One embodiment provides compounds having Structure (II): Formula (II) or a pharmaceutically acceptable salt, stereoisomer, tautomer, or prodrug thereof, wherein R^{1a}, R^{1b}, R², X, Y, and L are as defined herein. Methods associated with preparation and use of such compounds, pharmaceutical compositions comprising such compounds and methods to modulate the activity of MNK are also provided.

### CROSS-REFERENCE TO RELATED APPLICATIONS

This application claims the benefit of priority to U.S. Provisional Application No. 63/046,325, filed Jun. 30, 2020, which application is hereby incorporated by reference in its entirety.

### BACKGROUND

### Technical Field

The present disclosure describes compounds and methods useful as MNK inhibitors, useful for the treatment of neuropathic pain, Lupus, viral infection-induced pain, COVID-19 related acute respiratory distress syndrome (ARDS), nonalcoholic fatty liver disease (NAFLD), high fat diet induced obesity, Alzheimer's disease, Fragile X syndrome and related conditions. The present invention disclosure further describes a novel chemotype useful for the treatment of other disease types and other diseases that involve aberrant MNK activity.

### Description of the Related Art

The inadequate treatment of pain is a devastating health problem in the United States. One third of all Americans suffer from some form of chronic pain, and a third of these have pain that is resistant to current medical therapies. The economic impact of pain is equally large at approximately $100 billion annually. Opioid or narcotic analgesics, typified by morphine, are the most effective treatments for acute and chronic severe pain. However, their clinical utility is often hampered by the development of analgesic tolerance which requires escalating doses to achieve equivalent pain relief. Furthermore, these drugs are often ineffective for neuropathic pain treatment. This complex pathophysiological cycle represents a critical barrier to the quality of life of these patients due to the resulting drug-induced sedation, reduced physical activity, constipation, respiratory depression, high potential for addiction, and other side-effects.

Accordingly, there is a need to develop compounds that are effective for treating neuropathic pain. Embodiments of the present disclosure fulfill this need and provide further related advantages.

### BRIEF SUMMARY

In brief, embodiments of the present disclosure provide compounds, including pharmaceutically acceptable salts, stereoisomers, tautomers, and prodrugs thereof.

In one aspect, the disclosure provides compounds of Structure (I):

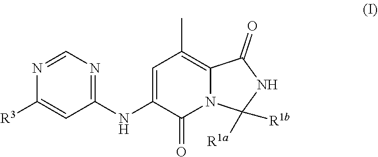

or a pharmaceutically acceptable salt, stereoisomer, tautomer, or prodrug thereof, wherein each of R^{1a}, R^{1b}, and R³ are as defined herein.

In another aspect, the disclosure provides compounds of Structure (II):

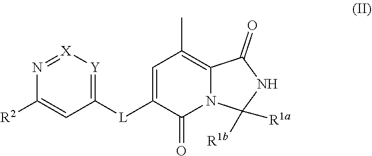

or a pharmaceutically acceptable salt, stereoisomer, tautomer, or prodrug thereof, wherein, R^{1a}, R^{1b}, R², X, Y, and L are as defined herein.

In another aspect, pharmaceutical compositions comprising the disclosed compounds, and methods of use of the same for treatment of diseases and disorders (e.g., neuropathic pain) are also provided.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is a graph of mean serum concentration over time of 4ET-03-009 in mice dosed orally with 10 mg/kg of an MNK inhibitor according to the present disclosure.

FIG. 2, left panel, is a Western blot showing eIF4E and phosphorylated eIF4E (p-eIF4E) in tissues harvested from the sciatic nerve, liver, brain, and dorsal root ganglion (CRD) of mice administered an MNK inhibitor (4-ET-03-009) or a control (vehicle).

FIG. 2, right panel, is a series of graphs quantifying mean peIF4E levels in the tissues in test and control mice.

FIG. 3 is a graph of mean serum concentration over time of 4ET-03-009 in mice dosed orally with 20 mg/kg.

FIG. 4 shows evaluation of compounds in the IL-6 evoked grimace test.

FIG. 5 depicts a comparison of effect size in the IL-6 evoked grimace test.

FIG. 6 is a graph showing the effect size in the IL-6 evoked grimace test vs. dose of 4ET-01-021.

FIG. 7A through 7P shows Western blot analysis in tissues from mice dosed with 4ET-01-021.

### DETAILED DESCRIPTION

The compounds of the present disclosure are capable of treating and preventing diseases associated with aberrant MNK activity, for example neuropathic pain, Lupus, viral infection-induced pain, COVID-19 related acute respiratory distress syndrome (ARDS), nonalcoholic fatty liver disease (NAFLD), high fat diet induced obesity, Alzheimer's disease, Fragile X syndrome. It has been discovered that MNK plays a key role in pain signaling. As a result, MNK is a potential drug target for the treatment of pain related disorders including neuropathic pain, as well as Lupus, viral infection-induced pain, COVID-19 related acute respiratory distress syndrome (ARDS), nonalcoholic fatty liver disease (NAFLD), high fat diet induced obesity, Alzheimer's disease, Fragile X syndrome.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The present disclosure is directed to MNK inhibitors and the treatment of diseases and disorders, including neuropathic pain, using the MNK inhibitors. The MNK inhibitors include the eFT508 derivatives described herein. These MNK inhibitors of the present disclosure have a different structure than eFT508 and may show comparative improvements in treatment of neuropathic pain, such as greater decrease in pain than a similar dose of eFT508, equal efficacy at a lower dose or at less frequent doses as compared to eFT508, or lower toxicity and better side-effect profile than eFT508. These comparative improvements in treatment of neuropathic pain may be measured directly, or by an assay indicative of the likelihood of such improvements. Many such suitable assays are disclosed herein. The MNK inhibitors may also have other improvements as compared to eFT508 that render them more clinically suitable for treatment of neuropathic pain, such as less blood brain barrier penetration, reducing central nervous system side effects. Similar improvements may be observed as compared to eFT508 with respect to other diseases and disorders, particularly those disclosed herein.

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean ±20%, ±10%, ±5% or ±1% of the indicated range, value, or structure, unless otherwise indicated. The use of the alternative (e.g., “or”) should be understood to mean either one, both, or any combination thereof of the alternatives.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Nitro” refers to the —NO₂ radical.

“Oxo” refers to the ═O substituent.

“Thiol” refers to the —SH substituent.

“Thioxo” refers to the ═S substituent.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching, but does not include carbon atoms belonging to any substituents. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds, having from two to twelve carbon atoms (C₂-C₁₂ alkenyl), two to eight carbon atoms (C₂-C₈ alkenyl) or two to six carbon atoms (C₂-C₆ alkenyl), or any value within these ranges, and which is attached to the rest of the molecule by a single bond, e.g., ethenyl, prop-1-enyl, but-1-enyl, pent-1-enyl, penta-1,4-dienyl, and the like. The number of carbons referred to relates to the carbon backbone and carbon branching, but does not include carbon atoms belonging to any substituents. Unless stated otherwise specifically in the specification, an alkenyl group is optionally substituted.

The term “alkynyl” refers to unsaturated straight or branched hydrocarbon radical, having 2 to 12 carbon atoms (C₂-C₁₂ alkynyl), two to nine carbon atoms (C₂-C₉ alkynyl), or two to six carbon atoms (C₂-C₆ alkynyl), or any value witin these ranges, and having at least one carbon-carbon triple bond. Examples of alkynyl groups may be selected from the group consisting of ethynyl, propargyl, but-1-ynyl, but-2-ynyl and the like. The number of carbons referred to relates to the carbon backbone and carbon branching, but does not include carbon atoms belonging to any substituents. Unless stated otherwise specifically in the specification, an alkynyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Aminyl” refers to a radical of the formula —NRₐR_{b}, where Rₐ is H or C₁-C₆ alkyl and R_{b} is C₁-C₆ alkyl as defined above. The C₁-C₆ alkyl portion of an aminyl group is optionally substituted unless stated otherwise.

“Aminylalkylcycloalkyl” refers to a radical of the formula —RₐR_{b}NR_{c}R_{d} where Rₐ is cycloalkyl as defined herein, R_{b} is C₁-C₆ alkyl, R_{c} is H or C₁-C₆ alkyl and R_{d} is C₁-C₆ alkyl as defined above. The cycloalkyl and each C₁-C₆ alkyl portion of an aminylalkylcycloalkyl group are optionally substituted unless stated otherwise.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of 7-electrons (for example, alternating double and single bonds) that is even but not a multiple of 4 (i.e., 4n+2π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, and pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene.

“Aryl” as used herein, includes a fused ring system that includes non-aromatic moieties. For example, in some embodiments, aryl may have one of the following structures:

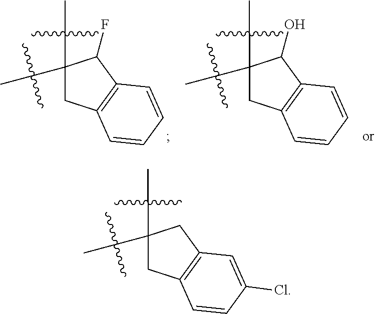 [SMILES: *C1(*)Cc2ccccc2C1F ; *C1(*)Cc2ccccc2C1O ; *C1(*)Cc2ccc(Cl)cc2C1]

Unless stated otherwise specifically in the specification, an aryl group is optionally substituted.

The term “arylalkyl” or “aralkyl” refers to the group -alkyl-aryl, where the alkyl and aryl groups are as defined herein. Aralkyl groups of the present disclosure are optionally substituted. Examples of arylalkyl groups include, for example, benzyl, 1-phenylethyl, 2-phenylethyl, 3-phenylpropyl, 2-phenylpropyl, fluorenylmethyl and the like.

“Cyanoalkyl” refers to an alkyl group comprising at least one cyano substituent. The —CN substituent may be on a primary, secondary or tertiary carbon. Unless stated otherwise specifically in the specification, a cyanoalkyl group is optionally substituted.

“Carbocyclic” or “carbocycle” refers to a ring system, wherein each of the ring atoms are carbon.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Alkylcycloalkyl” refers to a radical group of the formula —RₐR_{b} where Rₐ is a cycloalkyl group and R_{b} is an alkyl group as defined above. Unless otherwise stated specifically in the specification, an alkylcycloalkyl group is optionally substituted.

“Fused” refers to any ring structure described herein which is fused to another ring structure.

“Halo” refers to bromo, chloro, fluoro or iodo. “Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Halocycloalkyl” refers to a cycloalkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a halocycloalkyl group is optionally substituted.

“Haloalkylcycloalkyl” refers to a radical group of the formula —RₐR_{b} where Rₐ is a cycloalkyl group and R_{b} is a haloalkyl group as defined above. Unless otherwise stated specifically in the specification, a haloalkylcycloalkyl group is optionally substituted.

“Halocycloalkylalkyl” refers to a radical group of the formula —RₐR_{b} where Rₐ is an alkyl group and R_{b} is a halocycloalkyl group as defined above. Unless otherwise stated specifically in the specification, a halocycloalkylalkyl group is optionally substituted.

“Heterocyclylcycloalkyl” refers to a radical group of the formula —RₐR_{b} where Rₐ is a cycloalkyl group and R_{b} is a heterocyclyl group as defined herein. Unless otherwise stated specifically in the specification, a heterocyclylcycloalkyl group is optionally substituted.

“Hydroxylalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxylalkyl group is optionally substituted.

“Heterocyclyl,” “heterocyclic,” or “heterocycle” refer to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen and sulfur. Unless stated otherwise specifically in the specification, the heterocyclyl radical is partially or fully saturated and is a monocyclic, bicyclic, tricyclic or tetracyclic ring system, which may include fused, spirocyclic, and/or bridged ring systems. Nitrogen, carbon, and sulfur atoms in a heterocyclyl radical are optionally oxidized, and nitrogen atoms may be optionally quaternized. Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl. “Heterocyclyl” as used herein, includes a fused ring system that comprises additional non-heterocyclyl components. For example, in some embodiments, heterocyclyl may have one of the following structures:

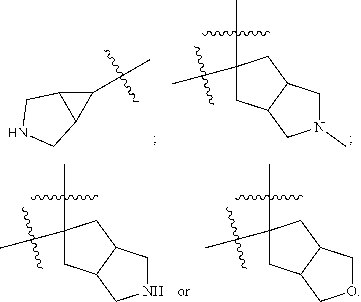 [SMILES: *C1C2CNCC12 ; *C1(*)CC2CN(C)CC2C1 ; *C1(*)CC2CNCC2C1 ; *C1(*)CC2COCC2C1]

Unless stated otherwise specifically in the specification, a heterocyclyl group is optionally substituted.

“Haloheterocyclyl” refers to a heterocyclyl group comprising at least one halo substituent. The halo substituent may be on a primary, secondary or tertiary carbon. Unless stated otherwise specifically in the specification, a haloheterocyclyl group is optionally substituted

“Haloheterocyclylalkyl” refers to a radical group of the formula —RₐR_{b} where Rₐ is an alkyl group and R_{b} is a haloheterocyclyl group as defined herein. Unless otherwise stated specifically in the specification, a haloheterocyclylalkyl group is optionally substituted.

“Heterocyclylalkyl” refers to a radical group of the formula —RₐR_{b} where Rₐ is an alkyl group and R_{b} is a heterocyclyl group as defined herein. Unless otherwise stated specifically in the specification, a heterocyclylalkyl group is optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen and sulfur, and at least one aromatic ring. Heteroaryl radicals may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). “Heteroaryl” as used herein, includes a fused ring system where the heteroatom (e.g., oxygen, sulfur, nitrogen, etc.) is not part of the aryl moiety. For example, in some embodiments, heteroaryl may have the following structure:

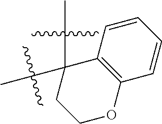 [SMILES: *C1(*)CCOc2ccccc21]

Unless stated otherwise specifically in the specification, a heteroaryl group is optionally substituted.

Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present disclosure fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

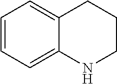

is, for the purposes of the present disclosure, considered a heterocyclic unit. 6,7-Dihydro-5H-cyclopentapyrimidine having the formula:

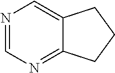

is, for the purposes of the present disclosure, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

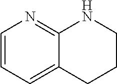

is, for the purposes of the present disclosure, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups are so indicated.

“Neuropathic pain” refers to pain that is not acutely caused by stimuli approaching or exceeding harmful intensity, such as harmful degrees of heat or cold, mechanical damage to a bodily tissue, chemical damage to a bodily tissue or exposure to a potentially harmful chemical, or, in some instances, acute inflammation (pain caused by these stimuli is referred to as nociceptive pain). Neuropathic pain results from disease or damage affecting neurons and is characterized by dysesthesia (abnormal sensations, allodynia (pain resulting from non-harmful and normally non-painful stimuli), or both. Neuropathic pain may be continuous, episodic, or both, at different times. Episodic neuropathic pain is often described as feeling like an electric shock. Neuropathic pain may also include burning or coldness, “pins and needles” sensations, numbness, itching, and any combination of these, including with or without electric shock sensations. Neuropathic pain may be acute or chronic. Acute neuropathic pain usually arises from nerve injury in trauma or chemotherapeutic or other drug treatments. Neuropathic pain is defined as chronic when it has lasted for more than 3 months.

“Patient” or “Subject” refers to an animal, such as a mammal, for example a human. The methods described herein can be useful in both human therapeutics and veterinary applications. In some embodiments, the subject is a mammal, and in some embodiments, the subject is human. Other subjects include mammals that do not tolerate opioids well or that are common pets or domesticated animals, such as dogs, cats, and horses.

“Mammal” includes humans and both domestic animals such as laboratory animals and household pets (e.g., cats, dogs, swine, cattle, sheep, goats, horses, rabbits), and non-domestic animals such as wildlife and the like.

“MNK” stands for mitogen-activated protein (MAP) kinases (MAPK) interacting kinases.

“Pharmaceutically acceptable” refers to molecular entities and compositions that do not produce adverse, allergic, or other untoward reactions when administered to an animal or a human.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“Drug” refers to a compound which is biologically active and provides a desired physiological effect following administration of a patient in need.

“Prodrug” is meant to indicate a compound that may be converted under physiological conditions or by solvolysis to a biologically active compound described herein (e.g., compounds of Structure (I) or (II)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

Additionally, embodiments of the present disclosure may provide prodrug of an MNK inhibitor. A prodrug is a compound that can be transformed to an active drug. In general, a prodrug is given to a patient and is then converted into a physiologically active form of the compound in vivo. In some instance, a prodrug may have a desired physiological effect. The prodrug of the present disclosure may include functional groups including esters, amides, phosphate ester, sulfonamide, or its combination thereof.

“Derivative” refers a compound that can be synthesized from a parent compound by replacement of one atom with another atom or group of atoms.

The term “effective amount” or “therapeutically effective amount” refers to that amount of a compound described herein that is sufficient to effect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

In some embodiments, pharmaceutically acceptable salts include quaternary ammonium salts such as quaternary amine alkyl halide salts (e.g., methyl bromide).

The term “in vivo” refers to an event that takes place in a subject's body.

Embodiments disclosed herein are also meant to encompass all pharmaceutically acceptable compounds of Structure (I) or (II).

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some aspects, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents or excipients therefor.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (i.e., compounds of Structure (I) or (II)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)-for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I) or (II) are a mixture of enantiomers or diastereomers. In other embodiments, the compounds of Structure (I) or (II) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Profesional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### Compounds

MNK inhibitors of the present disclosure are eFT508 derivatives. The structure of eFT508 is shown below:

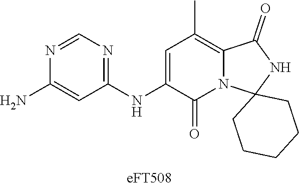 [SMILES: Cc1cc(Nc2cc(N)ncn2)c(=O)n2c1C(=O)NC21CCCCC1]

eFT508 is an orally bioavailable MNK1 and MNK2 inhibitor, with an IC₅₀ of 1-2 nM against both isoforms. Accordingly, in some embodiments MNK inhibitors of the present disclosure used as therapeutics may have an IC₅₀ of less than 1-2 nM, not inclusive, against MNK1, MNK2, or both.

In vitro, eFT508 reduces eIF4E phosphorylation dose-dependently at serine 209 with an IC₅₀ of between 2-16 nM in various tumor cell lines. Accordingly, in some embodiments, MNK inhibitors of the present disclosure for therapeutic use may exhibit lower IC₅₀ values than eFT508 in the same tumor cell lines. eFT508 has shown anti-proliferative activity against multiple diffuse large B cell lymphoma (DLBCL) cell lines. In some embodiments, MNK inhibitors of the present disclosure may also show anti-proliferative activity against these DLBCL cell lines at lower concentrations than eFT508. A cocrystal structure of eFT508 bound to MNK2 shows a key hydrogen bonding interaction with Lys161 and Met162 (J. Med. Chem. 2018, 61, 3516-3540 which is incorporated herein by reference). In certain embodiments, MNK inhibitors of the present disclosure may also show the same hydrogen bonding interaction with MNK2.

The present disclosure provides an MNK inhibitor that does not include eFT508. The MNK inhibitor may have the following Structure (Ia):

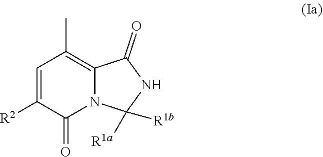

or a pharmaceutically acceptable salt, stereoisomer, tautomer, or prodrug thereof, wherein:

R^{1a} and R^{1b} are each independently alkyl. In some embodiments, R^{1a} and R^{1b} are the same. In certain embodiments, R^{1a} and R^{1b} are different. R^{1a} or R^{1b} may be alkyl groups, such as a methyl, ethyl, propyl, isopropyl, or tert-butyl group. The R^{1a} or R^{1b} substituent groups may be the same alkyl group, or different alkyl groups. For example, R^{1a} may be a methyl group, while R^{1b} may be an ethyl group. By way of another example, R^{1a} may be an isopropyl group, while R^{1b} may be a tert-butyl group. Any alkyl group combinations of substituents R^{1a} or R^{1b} may be used.

In some embodiments, R^{1a} and R^{1b} joint to form a cyclic moiety. In certain embodiments, the compound has the following Structure (Ib):

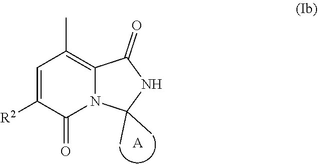

or a pharmaceutically acceptable salt, stereoisomer, tautomer, or prodrug thereof, wherein:
R^{1a} and R^{1b} may join together to form ring A.

The Structure (Ib), substituents R^{1a} or R^{1b} may together form a cyclic compound indicated as cyclic moiety A. For example, the cyclic moiety A of the Structure (Ib) may include a five-membered ring. The cyclic moiety A of the Structure (Ib) may be a non-substituted cyclic compound. For instance, the cyclic moiety A may be a non-substituted five-membered ring such as a cyclopentane. Further, the cyclic moiety A of the Structure (Ib) may have one or more alkyl substitutions. For example, the alkyl substitutions on the cyclic moiety A may include methyl, ethyl, propyl, isopropyl, cyclopropyl, or tert-butyl group. Substituted positions may be 2-, 3-, 4-, or 5-position of the cyclopentane. The degree of the substitutions may include mono-, di-, tri-, or tetra-substitutions. For instance, the cyclic moiety A may be 2,2,5,5-tetramethylcyclopentane. Synthetic routes may be used to install different substitution patterns on the cyclopentane ring. For example, the cyclic moiety A may be 3,3,4,4-tetramethylcyclopentane.

Additionally, the cyclic moiety A may have a fused ring. A part of the cyclic moiety A may include a fused benzene ring. For example, the cyclic moiety A may include the fused benzene ring with a cyclopentyl or cyclohexyl ring. For instance, the synthetic route to prepare the benzene fused cyclohexyl compound may involve the use of 1-tetralone. Further, the cyclic moiety A may include a fused cyclopentyl or cyclohexyl ring with other cyclic structures.

The cyclic moiety A may include a six-membered ring. The cyclic moiety A may be non-substituted cyclic moiety. For example, the cyclic moiety A may be a non-substituted six-membered ring such as a cyclohexane. Further, the cyclic moiety A may have one or more alkyl substitutions. For example, the alkyl substitutions on the cyclic moiety A may include methyl, ethyl, propyl, isopropyl, cyclopropyl, or tert-butyl group. The cyclic moiety A may have one or more heteroatom-containing substituents, such as alcohols, sulfonamides, or carboxylic acids. Substituted positions may be 2-, 3-, 4-, 5-, or 6-position of the cyclohexane. The degree of the substitutions may include mono-, di-, tri-, or tetra-substitutions. For instance, the cyclic moiety A may be 3,5-dimethylcyclohexane. Synthetic routes may be used to install different substitution patterns on the cyclohexane ring. For example, the cyclic moiety A may be 2,3,4,5,6-pentamethylcyclohexane.

The cyclic moiety A may include a heterocyclic compound. The heterocyclic compound is a cyclic compound that has atoms of at least two different elements such as a carbon and an oxygen atom. For example, the cyclic moiety A may be tetrahydropyran. The tetrahydropyran includes one oxygen atom and five carbon atoms in a six-membered ring. The heterocyclic compound may further be substituted with alkyl substituents or functional groups on various positions with various degrees of substitutions. It is noted that, while some of the structures shown in the present disclosure include an oxygen atom in a cyclic compound, such a structure is merely provided for illustrative purposes. Synthetic routes may be used to install different heteroatoms in the cyclic compounds. For example, the cyclic moiety A of the structure (Ib) may include piperidine (a nitrogen atom), phosphinate (a phosphorus atom), silinane (a silicon atom), or thiane (a sulfur atom).

The cyclic moiety A may be unsaturated. Unsaturated cyclic compounds may include aromatic cyclic compounds such as a benzene, pyridine, diazine, oxazine, dioxine, or thiazine. Alternatively, the cyclic moiety A may be saturated.

The cyclic moiety A may have one or more functional group substitutions. For example, the functional groups may include a hydroxyl, amine, amide, carboxylic acid, ether, or sulfonamide. Thus, the cyclic moiety A may include 4-hydroxyl cyclohexane, 4-carboxylic acid cyclohexane, 4-methoxyl cyclohexane, or 4-alkylsulfonamide cyclohexane. Substituted positions may be 2-, 3-, 4-, 5-, or 6-position of the cyclohexane. The degree of the substitutions may include mono-, di-, tri-, tetra-, or penta-substitutions. One or more functional groups may be installed on a heterocyclic compound with various substitution positions and degree.

The substituent R² of the structures (Ia) and (Ib) may include a nitrogen containing functional group. For example, the nitrogen containing functional group of the substituent R² may include amides, amidine, amines, amine oxides, azo, carbamates, carbodiimides, enamines, aromatic heterocycles, non-aromatic heterocycles, hydrazones, hydroxamic acids, imides, imines, nitriles, sulfonamide, or urea. For example, the aromatic heterocyles may include pyrrole, imidazole, pyrazole, thiazole, pyridine, pyridazine, pyrimidine, pyrazine, or triazine. The nitrogen containing functional group of substituent R² may be unsubstituted or substituted. For instance, a pyridazine may be substituted with an amine group at 3 position as shown in 4ET-004-006 hereinafter. In another instance, a pyridazine may be substituted with an amide containing a cyclopropyl ring at 3 position as shown in 4ET-004-003 hereinafter. The degree and location of substitution on the nitrogen containing functional group may differ. The nitrogen containing functional group of the substituent R² may be attached via an alkyl chain represented by —CₙH₂ₙ— where n is between zero and five. In this regard, the nitrogen containing functional groups of the substituent R² and the backbone structures (Ia) and (Ib) are separated by n carbon atoms.

Substituent R² of the structures (Ia) and (Ib) may include an aromatic heterocycle. For instance, in some embodiments, substituent R² may include 4-aminopyrimidinyl moiety. In some specific embodiments, the compound is a compound of Structure (Ic):

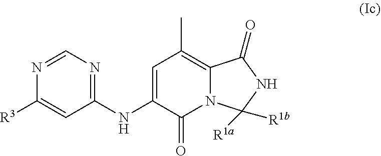

or pharmaceutically acceptable salt, stereoisomer, tautomer, or prodrug thereof, wherein:
R³ may include an amine.

In some embodiments, the amine is a primary amine. In some embodiments, R³ is —NH₂.

In some embodiments, R³ may include a secondary amine. When the amine is a secondary amine, R³ may further include a functional group at one end. For example, the functional group may include a hydroxyl, sulfonamide, carboxylic acid, ester, amine, amide, morpholine, piperazine, or thiomorpholine. The secondary amine and the functional group may be attached via an alkyl chain represented by —CₙH₂ₙ— where n is between one and five. Thus, the secondary amine of the substituent R³ and the functional group may be separated by n carbon atoms. For example, the secondary amine attached to a hydroxyl group separated by carbons atoms forms an aminoalcohol (HO—C₂H₄NH—), which is shown as examples 4ET-02-001, 4ET-03-004, 4ET-03-007, and 4ET-03-011 hereinafter. By way of another example, the secondary amine attached to a sulfonamide group separated by two carbon atoms forms amino sulfonamide (CH₃SO₂NHC₂H₄NH—), which is shown as examples 4ET-02-004, 4ET-03-012, 4ET-03-013, and 4ET-03-014 hereinafter.

The amine of substituent R³ may include a tertiary amine. The tertiary amine of the substituent R³ may be cyclic. The cyclic tertiary amine of the substituent R³ may be a part of saturated five-membered ring or six-membered ring. For example, the cyclic tertiary amine of the substituent R³ in a saturated five-membered ring may be pyrrolidine, imidazolidine, or pyrazolidine. The cyclic tertiary amine of the substituent R³ in a saturated six-membered ring may be piperidine or piperazine.

The tertiary amine may further include a functional group at one end. For example, the functional group may include a hydroxyl, sulfonamide, carboxylic acid, ester, amide, amine, morpholine, piperazine, or thiomorpholine. The tertiary amine and the functional group may be attached via an alkyl chain represented by —CₙH₂ₙ— where n is between one and five. Thus, the tertiary amine of the substituent R³ and the functional group may be separated by n carbon atoms.

The tertiary amine of the substituent R³ may be cyclic. The cyclic tertiary amine of the substituent R³ may be a part of unsaturated five-membered ring or six-membered ring. For example, the cyclic tertiary amine of the substituent R³ in an unsaturated five-membered ring may be pyrazole, imidazole, or oxazole. The cyclic tertiary amine of the substituent R³ in an unsaturated six-membered ring may be pyridine, diazine, triazine, or oxazine.

The amine of substituent R³ may also include an amide group. The amide group of substituent R³ may further include a functional group at one end. For example, the functional group may include a hydroxyl, sulfonamide, carboxylic acid, ester, amine, amide, morpholine, piperazine, or thiomorpholine.

The amide of the substituent R³ and the functional group may be attached via an alkyl chain represented by —CₙH₂ₙ— where n is between zero and five. Thus, the amide of the substituent R³ and the functional group may be separated by n carbon atoms. For example, the amide attached to morpholine group by one methylene forms morpholine amide, which is shown as examples 4ET-02-007, 4ET-03-027, and 4ET-03-028 hereinafter. The amide attached to morpholine group by two methylenes forms morpholine amide, which is shown as example 4ET-02-031 hereinafter. The amide of the substituent R³ may also be directly attached to one of the functional groups.

The amide of the substituent R³ may be directly attached to a cyclic structure. For example, the amide of the substituent R³ may be directly attached to cyclopropane. In this case, there is no carbon atom between the amide and cyclopropane. Structures with the amide group directly attached to cyclopropane as a part of the substituent R³ include 4ET-02-003, 4ET-02-009, 4ET-02-010, 4ET-02-011, 4ET-02-012, 4ET-02-016, 4ET-03-002, 4ET-03-009, 4ET-03-017, 4ET-03-019, 4ET-03-020, 4ET-03-023, 4ET-03-026, 4ET-03-034, and 4ET-04-003 hereinafter. Cyclopropanes may be unsubstituted or substituted with one or more functional groups. For instance, the substituted cyclopropanes may include fluorine, hydroxyl, hydroxylmethylene, alkyl, carboxylic acid, amine, aminomethylene, ester, ether, amide, sulfonamide, morpholine, piperazine, or thiomorpholine group attached to a cyclopropane ring. The substituted position on the cyclopropane where the functional group is attached may be the 1-, 2-, or 3-position. The functional group attached to the cyclopropane may have an additional alkyl chain (—CₙH₂ₙ—) between the functional group and the cyclopropane where n is between zero and 5. When n is equal to zero, there is no methylene between the functional group and the cyclopropane. Thus, the functional group may be directly attached to the cyclopropane on the 1-, 2-, or 3-position. Similarly, when n is equal to one, there is one methylene between the functional group and the cyclopropane. In this case, the functional group is one carbon away from the cyclopropane, which gives an extra degree of freedom to the structure. Structures with the amide group directly attached to substituted cyclopropane as a part of the substituent R³ include 4ET-02-009, 4ET-02-010, 4ET-02-011, 4ET-02-012, 4ET-02-016, 4ET-03-019, 4ET-03-020,4ET-03-023, 4ET-03-026, and 4ET-03-034 hereinafter.

The amide of the substituent R³ may be directly attached to cyclobutane. In this case, there is no carbon atom between the amide and cyclobutane. The cyclobutane may further have a functional group. For instance, the functional group may include hydroxyl, alkyl, carboxylic acid, amine, ester, ether, amide, sulfonamide, morpholine, piperazine, or thiomorpholine. The substituted position on the cyclobutane where the functional group is attached may be the 1-, 2-, 3-, or 4-position. The functional group may have an additional alkyl chain (CₙH₂ₙ) between the functional group and the cyclobutane where n is between zero and 5.

The cyclic structure that is attached to the amide via an alkyl chain or directly may include at least one heteroatom to form a heterocyclic compound. The heterocyclic compound may include a three-membered ring with one heteroatom or a four-membered ring with one heteroatom. For example, the three-membered ring with one heteroatom may include aziridines or ethylene oxide. By way of another example, the four-membered ring with one heteroatom may include azetidine or oxetane. Azetidine directly attached to the amide is shown for example in 4ET-02-017 hereinafter. As described above, functional groups may be attached to the heterocyclic compound. In the case of ethylene oxide (epoxide), Sharpless epoxidation may be used to generate chiral epoxides.

While the examples herein only have a monosubstitution on the cyclic structure, such a configuration is merely provided for illustrative purposes. Embodiments of the present disclosure include disubstituted cyclic structures as well. For example, a total of two amine groups may be attached to the cyclopropane; a first amine group may be attached to 1-position of cyclopropane, while a second amine group is attached to 2-position of cyclopropane.

The amide of the substituent R³ may be a reverse amide. Instead of a nitrogen atom of the amide of the substituent R³ being directly attached to the structure (Ic), a carbon atom of the amide of the substituent R³ may be attached to the structure (Ic). The reverse amide attached to the structure (Ic) is shown for example in 4ET-03-024 hereinafter. Embodiments of the present disclosure described above including the amide in the substituent R³ may also be replaced with a reverse amide. For instance, the amide group of examples such as 4ET-02-003, 4ET-02-009, 4ET-02-010, 4ET-02-011, 4ET-02-012, 4ET-02-016, 4ET-03-002, 4ET-03-009, 4ET-03-017, 4ET-03-019, 4ET-03-020, 4ET-03-023, 4ET-03-026, 4ET-03-034, 4ET-04-003, 4ET-02-007, 4ET-03-027, 4ET-03-028, and 4ET-02-031 may be replaced with a reverse amide.

The structure (Ic) may be equipped with an amide analog of the substituent R³. For example, a thioamide group may be used instead of the amide group shown in 4ET-02-013 hereinafter. Similar to the amide substituent, the thioamide group may be replaced with a reverse thioamide. In this regard, instead of a nitrogen atom of the thiamide of the substituent R³ being directly attached to the structure (Ic), a carbon atom of the thioamide of the substituent R³ may be attached to the structure (Ic).

Additionally, other amide analogs of the substituent R³ may be used for the structure (Ic). For example, a urea group may be used instead of the amide group shown in 4ET-02-015 hereinafter. By way of another example, a thiourea group may be used instead of the amide group. An amide, a reverse amide, a thioamide, a reverse thioamide, a urea, and a thiourea as a part of the substituent R³ are interchangeable in the structure (Ic).

In some MNK inhibitors of the present disclosure, the 4-aminopyrimidine moiety in structure (Ic) may be modified. The pyrimidine moiety and the parent structure as shown in the structure (Ia) or (Ib) are connected via the amine linker (—NH—) in the structure (Ic). The amine linker may be extended. For example, the amine linker may include additional alkyl chain (—CₙH₂ₙ—) between the amine and pyrimidine moiety where n is between one and five. For instance, one extra carbon atom (n=1) may be added, such that the amine linker and pyrimidine are one carbon away from the parent structure, as shown in 4ET-04-004, which gives the structure (Ic) more structural flexibility via an extra degree of freedom. One carbon extension, which is an insertion of a methylene unit, between the amine and the pyrimidine moiety provides a benzylpyrimidine moiety. By way of another example, the amine linker may include additional alkyl chain (—CₙH₂ₙ—) between the amine and the parent structure shown as the structure (Ia) or (Ib) where n is between one and five. For instance, one extra carbon atom (n=1) may be added, as shown in 4ET-04-015, such that the amine linker and the parent structure shown as the structure (Ia) or (Ib) are one carbon away from the parent structure. One carbon extension, which is an insertion of a methylene unit, between the amine and the structure (Ia) or (Ib) provides a methylaminopyrimidine moiety. In this regard, methylene units may be added both sides of the amine linker of the structure (Ic). Amine linker extension with extra methylene units may be used in conjunction with any of the other variations of structures (Ia), (Ib), and (Ic) disclosed herein.

Additionally, the pyrimidine moiety in the structure (Ic) may be modified to substitute a different unsaturated six-membered ring with two nitrogen atoms isomer, such as 1,2-diazine (pyridazine) or 1,4-diazine (pyrazine). For example, 1,2-diazine (pyridazine) may be used instead of 1,3-diazine (pyrimidine) in the structure (Ic) shown in example 4ET-04-003 and 4ET-04-006 hereinafter. These modifications may be used in conjunction with any of the other variations of structures (Ia), (Ib), and (Ic) disclosed herein.

Pyrimidine in the structure (Ic) may be replaced with a five-membered heterocyclic compound. Pyrimidine is a six-membered heterocyclic compound with two nitrogen atoms. In general, five-membered heterocyclic compounds have different chemical and physical properties than the six-membered heterocyclic compounds. Some MNK inhibitors of the present disclosure may take advantage of such differences between five- and six-membered heterocyclic compounds. For example, the five-membered heterocyclic compound may include nitrogen and sulfur atoms. For instance, the five-membered heterocyclic compound with N and S may include thiazole as shown in example 4ET-04-001 hereinafter. By way of another example, the five-membered heterocyclic compound with S may include thiophene. The five-membered heterocyclic compound may include nitrogen and oxygen atoms. For instance, the five-membered heterocyclic compound with N and O may include oxazole or isoxazole. Yet in another example, the five-membered heterocyclic compound may include two nitrogen atoms. For instance, the five-membered heterocyclic compound with two nitrogen atoms may include imidazole or pyrazole. These modifications may be used on conjunction with any of the other variations of structures (Ia), (Ib), and (Ic) disclosed herein.

As examples of how various modifications disclosed herein may be used in combination with one another, the amine linker with additional carbon atom may be attached to a pyridazine moiety and the pyridazine moiety may be connected to the pyridone scaffold with an amine or sulfonamide. By way of another example, the amine linker with additional carbon atom may be attached to a pyridazine moiety and the pyridazine moiety may be directly connected to an amino group.

In some MNK inhibitors of the present disclosure, the 4-aminopyrimidine moiety and a parent structure, for example, a pyridone moiety in structure (Ic) may be attached via other nitrogen containing linkers. The pyrimidine moiety and the parent structure as shown in the structure (Ia) or (Ib) are connected via the amine linker (—NH—) in the structure (Ic). Embodiments of the present disclosure may be configured to install an amide group between the 4-aminopyrimidine moiety and the parent structure. This can be synthesized by using an amide containing starting material in Buchwald-Hartwig amination described in Example 1—MNK inhibitor synthesis. For example, the resulting MNK inhibitor may include an amide as shown in example 4ET-04-013 or a reverse amide as shown in example 4ET-04-014 hereinbelow between the 4-aminopyrimidine moiety and the parent structure.

Further, embodiments of the present disclosure may be configured to install a sulfonamide group between the 4-aminopyrimidine moiety and the parent structure. This can be synthesized by using a sulfonamide containing starting material in Buchwald-Hartwig amination described in Example 1—MNK inhibitor synthesis. Another approach involves the use of a sulfonyl chloride reagent or intermediate. For example, the resulting MNK inhibitor may include a sulfonamide as shown in examples 4ET-04-010 and 4ET-04-011 or a reverse sulfonamide as shown in example 4ET-04-012 hereinbelow between the 4-aminopyrimidine moiety and the parent structure.

Additionally, embodiments of the present disclosure may be configured to install an ether group between the 4-aminopyrimidine moiety and the parent structure. This can be synthesized by using an alcohol containing starting material in Buchwald-Hartwig amination described in Example 1—MNK inhibitor synthesis. Another approach involves using an alcohol containing starting material in an Ullmann-type coupling reaction.

The substituents R^{1a} or R^{1b} of the structure (Ic) may be alkyl groups, as discussed hereinabove in the structure (Ia). Alternatively, the substituents R^{1a} or R^{1b} of the structure (Ic) may together form a cyclic compound indicated as a ring structure A below. The detailed discussion of the ring structure A of the structure (Ib) may also apply to the structure (Id):

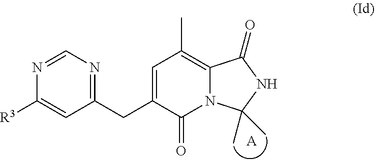

or pharmaceutically acceptable salt, stereoisomer, tautomer, or prodrug thereof, wherein:
R³ may include an amine.

One embodiment provides a compound having the following Structure (II):

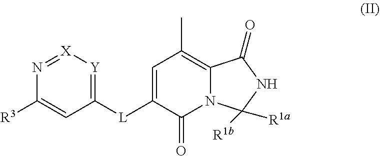

or a pharmaceutically acceptable salt, stereoisomer, tautomer, or prodrug thereof, wherein
R^{1a} is C₁-C₆ alkyl or aryl;
R^{1b} is C₁-C₆ alkyl or aryl,
   or R^{1a} and R^{1b}, together with the carbon to which they are both attached, join to form cycloalkyl, cycloalkenyl, heterocyclyl, aryl, or heteroaryl;
R² is —NHR^{3a}, —NHC(═O)R^{3b}, —NHC(═S)R^{3b}, or —C(═O)R^{3c};
R^{3a} is hydrogen, C₁-C₆ alkyl, or C₃-C₆ cycloalkyl, each of which is optionally substituted with one or more substituents selected from the group consisting of hydroxyl, C₃-C₆ cycloalkyl, —NHS(O)₂CH₃, heterocyclyl, —C(═O)OH, —C(═O)N(R^{3d})R^{3d}, or —N(R^{3d})R^{3d};
R^{3b} is C₁-C₆ alkyl, C₃-C₆ cycloalkyl, or heterocyclyl each of which is optionally substituted with one or more substituents selected from the group consisting of hydroxyl, halo, C₁-C₆ alkyl, C₃-C₆ cycloalkyl, —NHS(O)₂CH₃, —N(R^{3d})R^{3d}, heterocyclyl, —C(═O)OH, —C(═O)N(R^{3d})R^{3d}, —NHC(═O)CH₃, —CH₂C(═O)OH,
R^{3c} is —N(R^{3d})R^{3d} or heterocyclyl;
R^{3d} is, at each occurrence, independently hydrogen, C₁-C₆ alkyl, or C₃-C₆ cycloalkyl;
L is —NH— or —CH₂NH—; and
X is N and Y is CH or X is CH and Y is N,
   provided that:
when R^{1a} and R^{1b} are both —CH₃ or when R^{1a} and R^{1b} join to form a 5- or 6-membered cycloalkyl or heterocyclyl, then R² does not have the following structure:
—NH₂ or

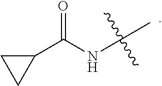 [SMILES: *NC(=O)C1CC1]

In some embodiments, R^{1a} is C₁-C₆ alkyl. In some embodiments, R^{1a} is methyl. In certain embodiments, R^{1a} is aryl. In certain embodiments, R^{1a} is phenyl.

In certain specific embodiments, R^{1b} is C₁-C₆ alkyl. In some embodiments, R^{1b} is methyl. In some embodiments, R^{1a} and R^{1b}, together with the carbon to which they are both attached, join to form cycloalkyl. In more specific embodiments, the cycloalkyl is cyclopentyl or cyclohexyl. In some embodiments, R^{1a} and R^{1b}, together with the carbon to which they are both attached, join to form cycloalkenyl. In some embodiments, the cycloalkenyl is cyclopentenyl, cyclohexenyl, or cycloheptenyl. In certain specific embodiments, R^{1a} and R^{1b}, together with the carbon to which they are both attached, join to form heterocyclyl. In some specific embodiments, R^{1a} and R^{1b}, together with the carbon to which they are both attached, join to form aryl. In some embodiments, R^{1a} and R^{1b}, together with the carbon to which they are both attached, join to form heteroaryl.

In more specific embodiments, the compound has one of the following structures:

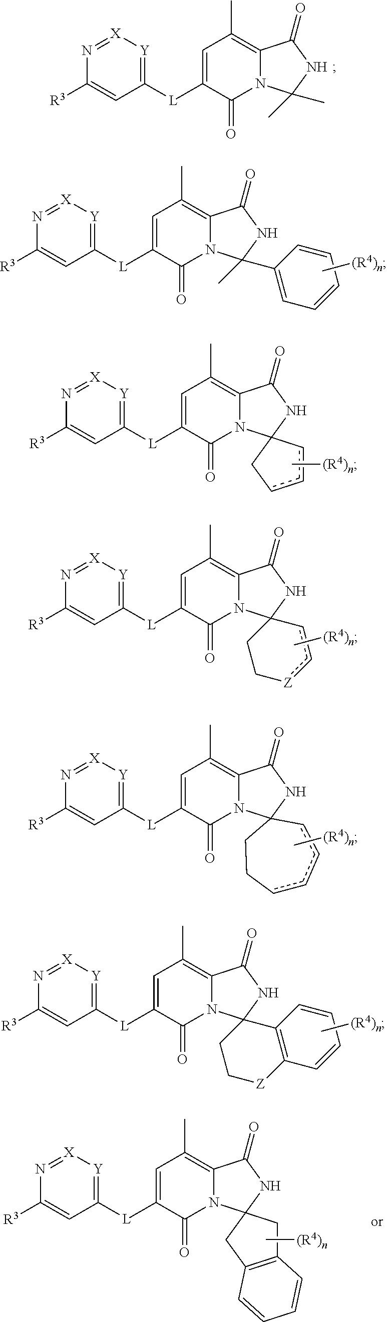

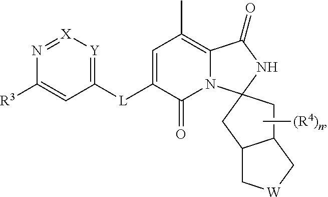

or a pharmaceutically acceptable salt, stereoisomer, tautomer, or prodrug thereof, wherein
[symbol] indicates a double or single bond;
R⁴ is, at each occurrence, independently C₁-C₆ alkyl, C₃-C₆ cycloalkyl, halo, haloalkyl, hydroxyl, —NHS(O)₂CH₃, or —C(O)OH,
   or two R⁴, together with the carbon to which they are both attached, join to form a cycloalkyl;
W is N or O;
Z is C or O; and
n is 0, 1, 2, 3, or 4.

In some embodiments, n is 0, 1, or 2. In some more specific embodiments, only one location depicted with [symbol] is a double bond and the rest are single bonds. In some embodiments, the compound has the following structure:

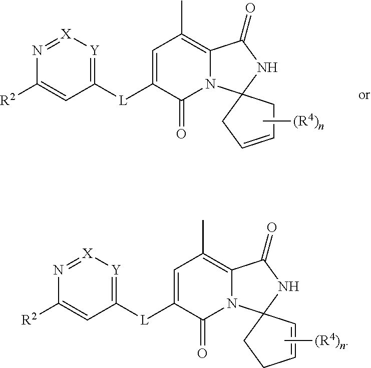

In some more specific embodiments, the compound has the following structure:

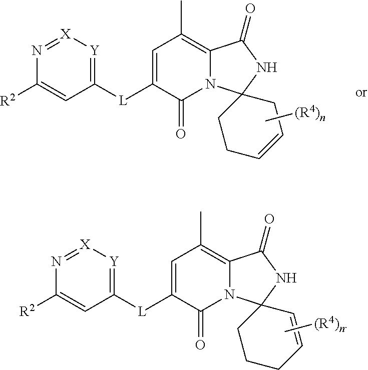

In some embodiments, the compound has one of the following structures:

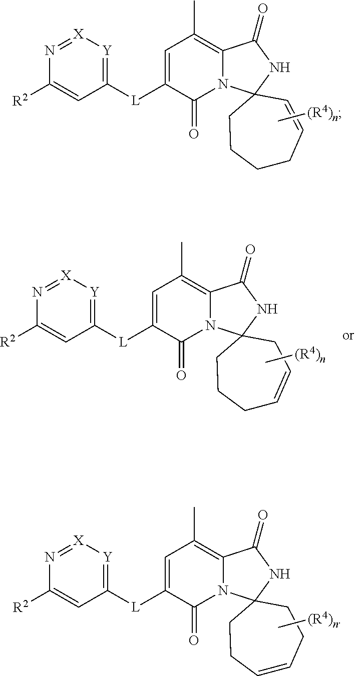

In more specific embodiments, R² is —NHR^{3a}. In more specific embodiments, R² has one of the following structures:

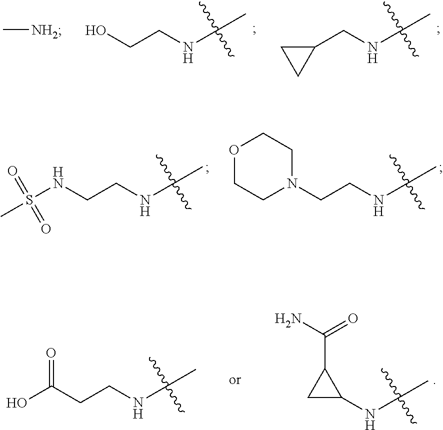

In some embodiments, R² is —NHC(═O)R^{3b}. In more specific embodiments, R² has one of the following structures:

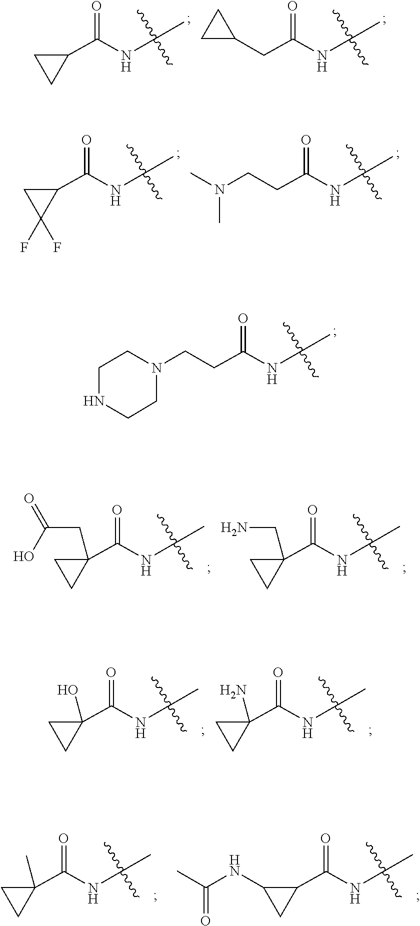

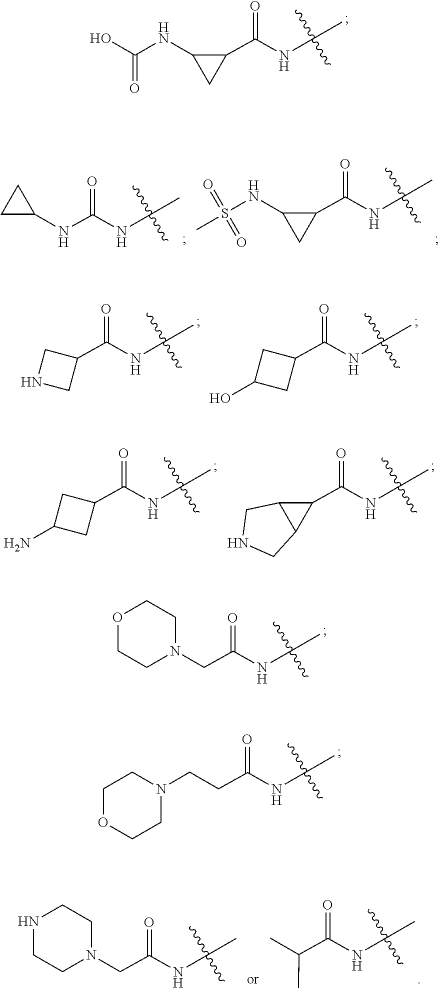

In certain embodiments, R² is —NHC(═S)R^{3b}. In certain embodiments, R² has the following structure:

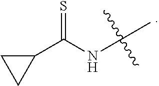 [SMILES: *NC(=S)C1CC1 ; "."]

In certain embodiments, R² is —C(═O)R^{3c}. In some embodiments, R² has one of the following structures:

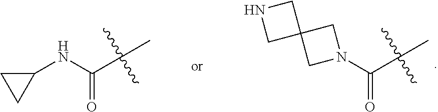 [SMILES: *C(=O)NC1CC1 ; "or" ; *C(=O)N1CC2(CNC2)C1 ; "."]

In some embodiments, R² has one of the following structures:

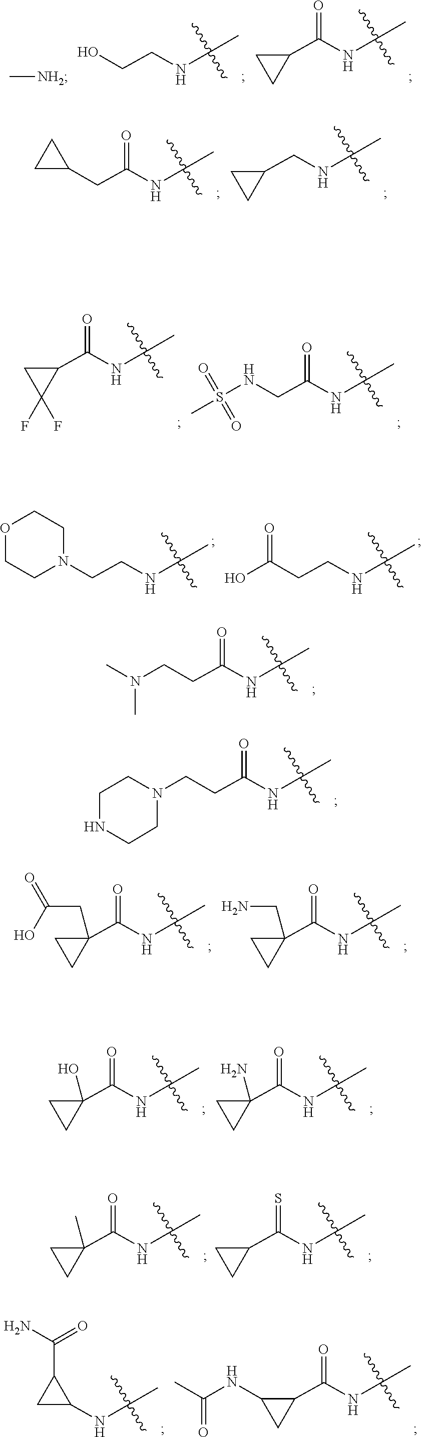

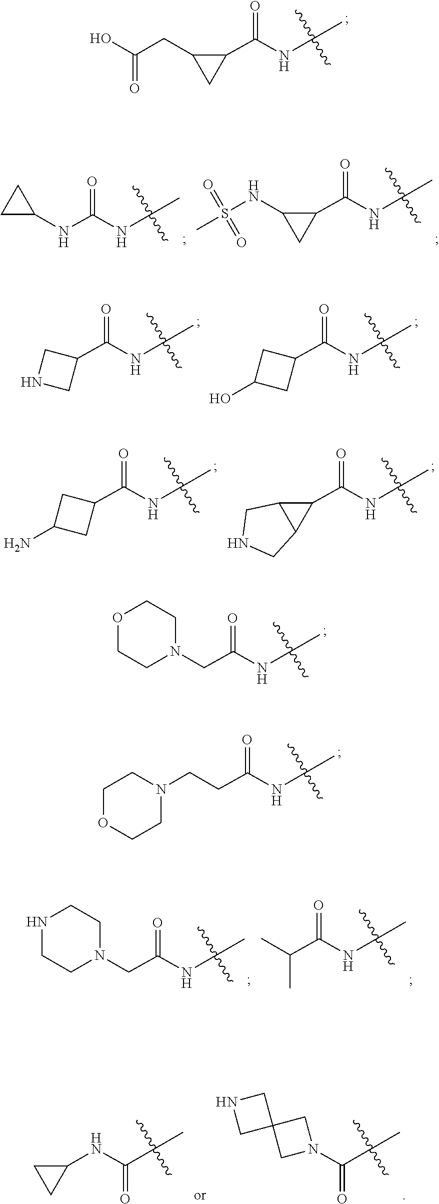

In certain specific embodiments, R² has one of the following structures:
—NH₂ or

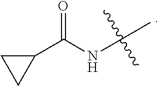 [SMILES: *NC(=O)C1CC1 ; "."]

In some embodiments, X is CH and Y is N. In certain embodiments, X is N and Y is CH. In some embodiments, L is —NH—. In more embodiments, L is —CH₂NH—.

In some embodiments, R^{3a} is a branched C₁-C₆ alkyl. In some embodiments, R^{3a} is iso-propyl.

In various different embodiments, the compound has one of the structures set forth in Table 1 below, or a pharmaceutically acceptable salt, stereoisomer, tautomer, or prodrug thereof. Compounds in Table 1 were prepared as described in the Examples and/or methods known in the art.

TABLE 1
Representative Structure

| No | Structure |
|---|---|
| 4ET- 01- 001 | 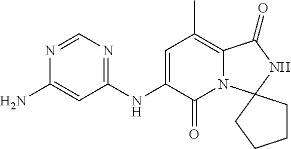 [SMILES: Cc1cc(Nc2cc(N)ncn2)c(=O)n2c1C(=O)NC21CCCC1] |
| 4ET- 01- 002 | 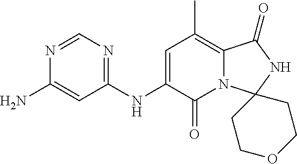 [SMILES: Cc1cc(Nc2cc(N)ncn2)c(=O)n2c1C(=O)NC21CCOCC1] |
| 4ET- 01- 003 | 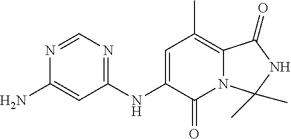 [SMILES: Cc1cc(Nc2cc(N)ncn2)c(=O)n2c1C(=O)NC2(C)C] |
| 4ET- 01- 004 | 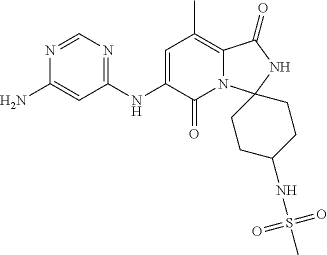 [SMILES: Cc1cc(Nc2cc(N)ncn2)c(=O)n2c1C(=O)NC21CCC(NS(C)(=O)=O)CC1] |
| 4ET- 01- 005 | 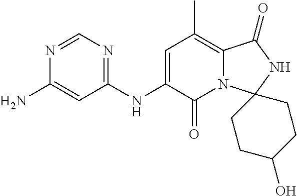 [SMILES: Cc1cc(Nc2cc(N)ncn2)c(=O)n2c1C(=O)NC21CCC(O)CC1] |
| 4ET- 01- 010A | 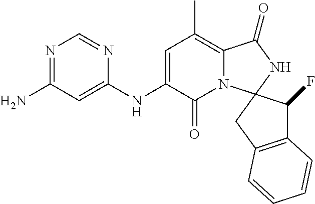 [SMILES: Cc1cc(Nc2cc(N)ncn2)c(=O)n2c1C(=O)NC21Cc2ccccc2[C@@H]1F] |
| 4ET- 01- 010B | 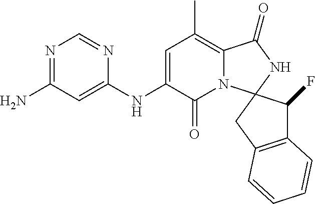 [SMILES: Cc1cc(Nc2cc(N)ncn2)c(=O)n2c1C(=O)NC21Cc2ccccc2[C@@H]1F] |
| 4ET- 01- 014A | 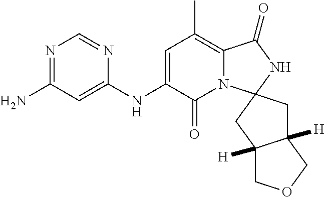 [SMILES: Cc1cc(Nc2cc(N)ncn2)c(=O)n2c1C(=O)NC21C[C@H]2COC[C@H]2C1] |
| 4ET- 01- 014B | 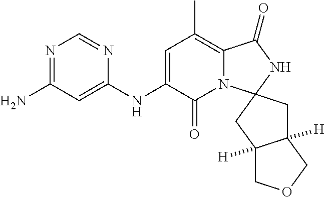 [SMILES: Cc1cc(Nc2cc(N)ncn2)c(=O)n2c1C(=O)NC21C[C@H]2COC[C@H]2C1] |
| 4ET- 01- 021 | 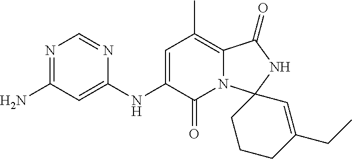 [SMILES: CCC1=CC2(CCC1)NC(=O)c1c(C)cc(Nc3cc(N)ncn3)c(=O)n12] |
| 4ET- 01- 058 | 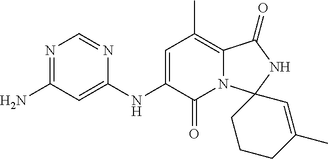 [SMILES: CC1=CC2(CCC1)NC(=O)c1c(C)cc(Nc3cc(N)ncn3)c(=O)n12] |
| 4ET- 02- 001 | 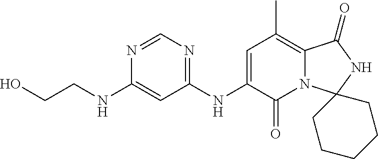 [SMILES: Cc1cc(Nc2cc(NCCO)ncn2)c(=O)n2c1C(=O)NC21CCCCC1] |
| 4ET- 02- 002 | 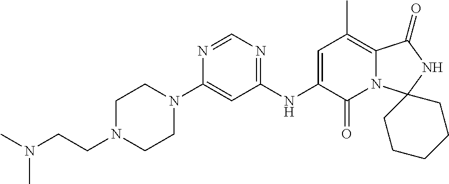 [SMILES: Cc1cc(Nc2cc(N3CCN(CCN(C)C)CC3)ncn2)c(=O)n2c1C(=O)NC21CCCCC1] |
| 4ET- 02- 003 | 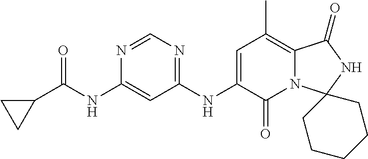 [SMILES: Cc1cc(Nc2cc(NC(=O)C3CC3)ncn2)c(=O)n2c1C(=O)NC21CCCCC1] |
| 4ET- 02- 004 | 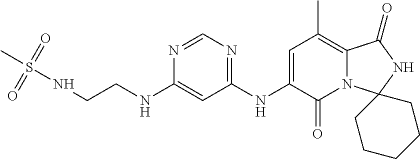 [SMILES: Cc1cc(Nc2cc(NCCNS(C)(=O)=O)ncn2)c(=O)n2c1C(=O)NC21CCCCC1] |
| 4ET- 02- 005 | 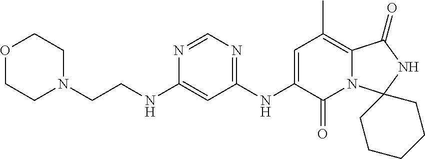 [SMILES: Cc1cc(Nc2cc(NCCN3CCOCC3)ncn2)c(=O)n2c1C(=O)NC21CCCCC1] |
| 4ET- 02- 006 | 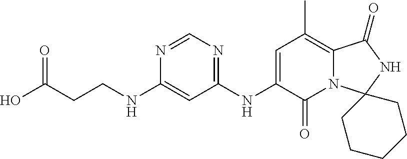 [SMILES: Cc1cc(Nc2cc(NCCC(=O)O)ncn2)c(=O)n2c1C(=O)NC21CCCCC1] |
| 4ET- 02- 007 | 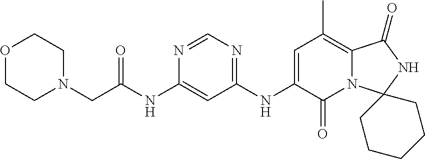 [SMILES: Cc1cc(Nc2cc(NC(=O)CN3CCOCC3)ncn2)c(=O)n2c1C(=O)NC21CCCCC1] |
| 4ET- 02- 008 | 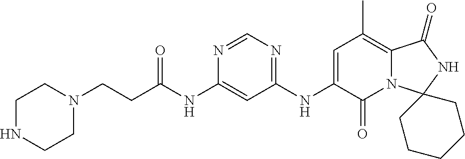 [SMILES: Cc1cc(Nc2cc(NC(=O)CCN3CCNCC3)ncn2)c(=O)n2c1C(=O)NC21CCCCC1] |
| 4ET- 02- 009 | 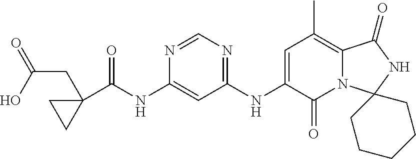 [SMILES: Cc1cc(Nc2cc(NC(=O)C3(CC(=O)O)CC3)ncn2)c(=O)n2c1C(=O)NC21CCCCC1] |
| 4ET- 02- 010 | 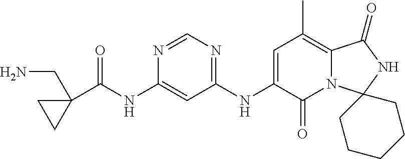 [SMILES: Cc1cc(Nc2cc(NC(=O)C3(CN)CC3)ncn2)c(=O)n2c1C(=O)NC21CCCCC1] |
| 4ET- 02- 011 | 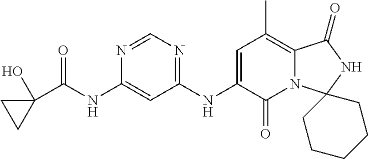 [SMILES: Cc1cc(Nc2cc(NC(=O)C3(O)CC3)ncn2)c(=O)n2c1C(=O)NC21CCCCC1] |
| 4ET- 02- 012 | 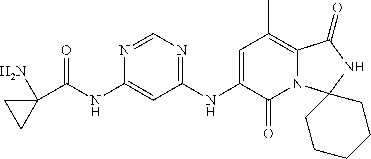 [SMILES: Cc1cc(Nc2cc(NC(=O)C3(N)CC3)ncn2)c(=O)n2c1C(=O)NC21CCCCC1] |
| 4ET- 02- 013 | 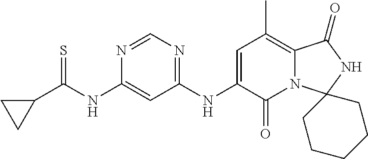 [SMILES: Cc1cc(Nc2cc(NC(=S)C3CC3)ncn2)c(=O)n2c1C(=O)NC21CCCCC1] |
| 4ET- 02- 014 | 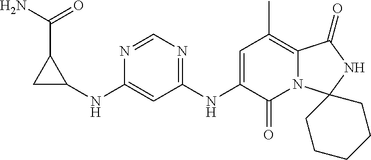 [SMILES: Cc1cc(Nc2cc(NC3CC3C(N)=O)ncn2)c(=O)n2c1C(=O)NC21CCCCC1] |
| 4ET- 02- 015 | 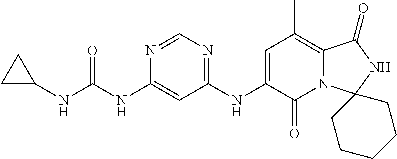 [SMILES: Cc1cc(Nc2cc(NC(=O)NC3CC3)ncn2)c(=O)n2c1C(=O)NC21CCCCC1] |
| 4ET- 02- 016 | 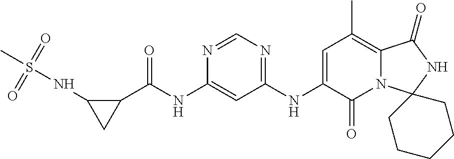 [SMILES: Cc1cc(Nc2cc(NC(=O)C3CC3NS(C)(=O)=O)ncn2)c(=O)n2c1C(=O)NC21CCCCC1] |
| 4ET- 02- 017 | 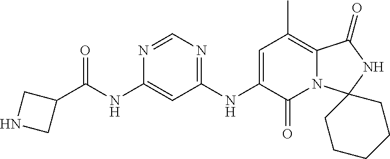 [SMILES: Cc1cc(Nc2cc(NC(=O)C3CNC3)ncn2)c(=O)n2c1C(=O)NC21CCCCC1] |
| 4ET- 02- 018 | 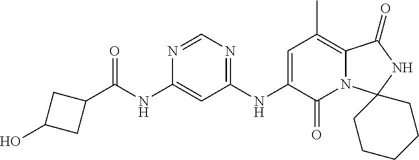 [SMILES: Cc1cc(Nc2cc(NC(=O)C3CC(O)C3)ncn2)c(=O)n2c1C(=O)NC21CCCCC1] |
| 4ET- 02- 019 | 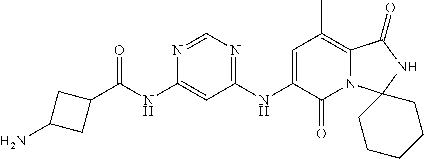 [SMILES: Cc1cc(Nc2cc(NC(=O)C3CC(N)C3)ncn2)c(=O)n2c1C(=O)NC21CCCCC1] |
| 4ET- 02- 020 | 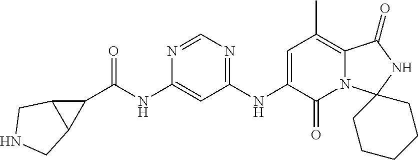 [SMILES: Cc1cc(Nc2cc(NC(=O)C3C4CNCC43)ncn2)c(=O)n2c1C(=O)NC21CCCCC1] |
| 4ET- 02- 021 | 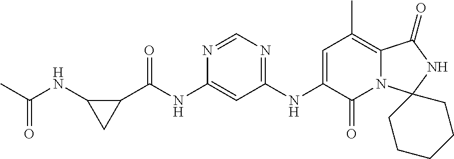 [SMILES: CC(=O)NC1CC1C(=O)Nc1cc(Nc2cc(C)c3n(c2=O)C2(CCCCC2)NC3=O)ncn1] |
| 4ET- 02- 022 | 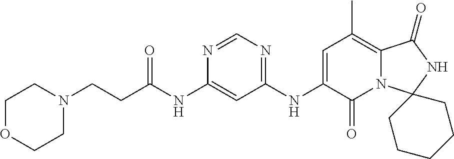 [SMILES: Cc1cc(Nc2cc(NC(=O)CCN3CCOCC3)ncn2)c(=O)n2c1C(=O)NC21CCCCC1] |
| 4ET- 02- 023 | 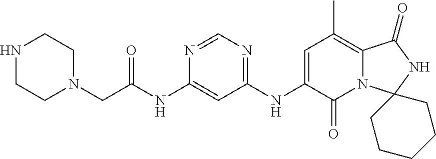 [SMILES: Cc1cc(Nc2cc(NC(=O)CN3CCNCC3)ncn2)c(=O)n2c1C(=O)NC21CCCCC1] |
| 4ET- 03- 001 | 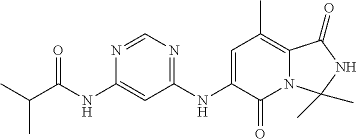 [SMILES: Cc1cc(Nc2cc(NC(=O)C(C)C)ncn2)c(=O)n2c1C(=O)NC2(C)C] |
| 4ET- 03- 002 | 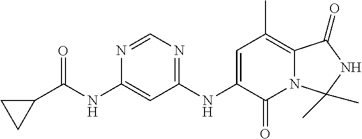 [SMILES: Cc1cc(Nc2cc(NC(=O)C3CC3)ncn2)c(=O)n2c1C(=O)NC2(C)C] |
| 4ET- 03- 003 | 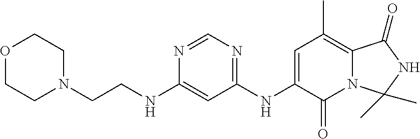 [SMILES: Cc1cc(Nc2cc(NCCN3CCOCC3)ncn2)c(=O)n2c1C(=O)NC2(C)C] |
| 4ET- 03- 004 | 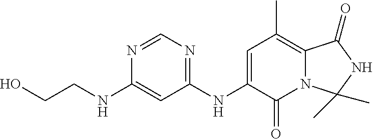 [SMILES: Cc1cc(Nc2cc(NCCO)ncn2)c(=O)n2c1C(=O)NC2(C)C] |
| 4ET- 03- 005 | 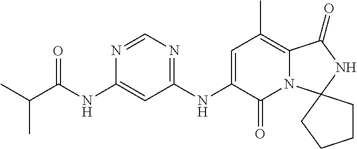 [SMILES: Cc1cc(Nc2cc(NC(=O)C(C)C)ncn2)c(=O)n2c1C(=O)NC21CCCC1] |
| 4ET- 03- 006 | 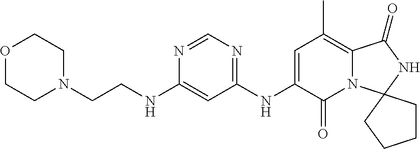 [SMILES: Cc1cc(Nc2cc(NCCN3CCOCC3)ncn2)c(=O)n2c1C(=O)NC21CCCC1] |
| 4ET- 03- 007 | 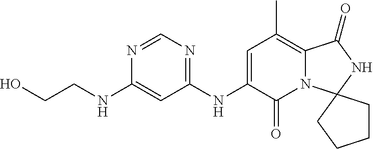 [SMILES: Cc1cc(Nc2cc(NCCO)ncn2)c(=O)n2c1C(=O)NC21CCCC1] |
| 4ET- 03- 008 | 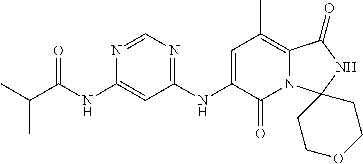 [SMILES: Cc1cc(Nc2cc(NC(=O)C(C)C)ncn2)c(=O)n2c1C(=O)NC21CCOCC1] |
| 4ET- 03- 009 | 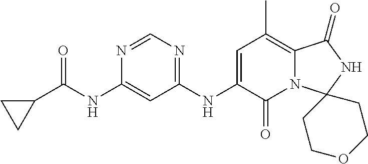 [SMILES: Cc1cc(Nc2cc(NC(=O)C3CC3)ncn2)c(=O)n2c1C(=O)NC21CCOCC1] |
| 4ET- 03- 010 | 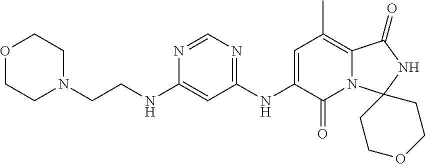 [SMILES: Cc1cc(Nc2cc(NCCN3CCOCC3)ncn2)c(=O)n2c1C(=O)NC21CCOCC1] |
| 4ET- 03- 011 | 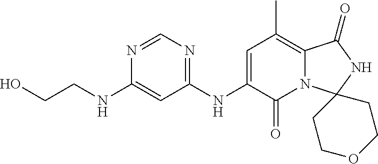 [SMILES: Cc1cc(Nc2cc(NCCO)ncn2)c(=O)n2c1C(=O)NC21CCOCC1] |
| 4ET- 03- 012 | 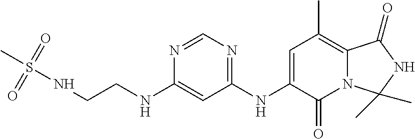 |
| 4ET- 03- 013 | 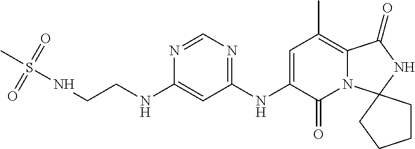 [SMILES: Cc1cc(Nc2cc(NCCNS(C)(=O)=O)ncn2)c(=O)n2c1C(=O)NC21CCCC1] |
| 4ET- 03- 014 | 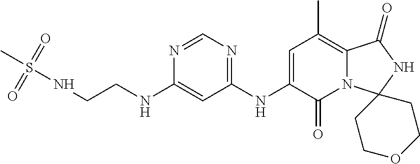 [SMILES: Cc1cc(Nc2cc(NCCNS(C)(=O)=O)ncn2)c(=O)n2c1C(=O)NC21CCOCC1] |
| 4ET- 03- 015 | 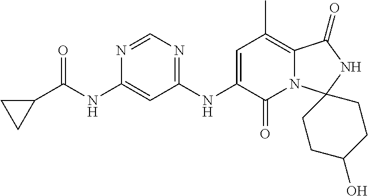 [SMILES: Cc1cc(Nc2cc(NC(=O)C3CC3)ncn2)c(=O)n2c1C(=O)NC21CCC(O)CC1] |
| 4ET- 03- 016 | 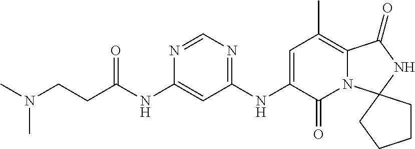 [SMILES: Cc1cc(Nc2cc(NC(=O)CCN(C)C)ncn2)c(=O)n2c1C(=O)NC21CCCC1] |
| 4ET- 03- 017 | 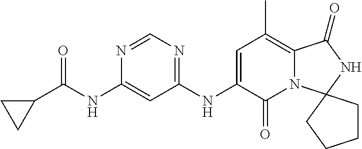 [SMILES: Cc1cc(Nc2cc(NC(=O)C3CC3)ncn2)c(=O)n2c1C(=O)NC21CCCC1] |
| 4ET- 03- 018 | 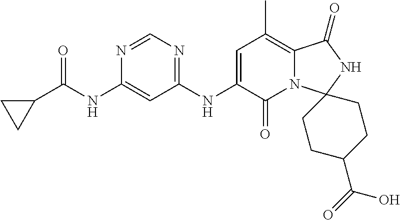 [SMILES: Cc1cc(Nc2cc(NC(=O)C3CC3)ncn2)c(=O)n2c1C(=O)NC21CCC(C(=O)O)CC1] |
| 4ET- 03- 019 | 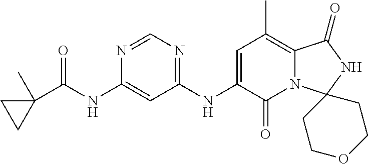 [SMILES: Cc1cc(Nc2cc(NC(=O)C3(C)CC3)ncn2)c(=O)n2c1C(=O)NC21CCOCC1] |
| 4ET- 03- 020 | 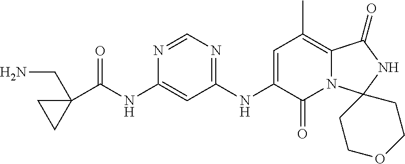 [SMILES: Cc1cc(Nc2cc(NC(=O)C3(CN)CC3)ncn2)c(=O)n2c1C(=O)NC21CCOCC1] |
| 4ET- 03- 021 | 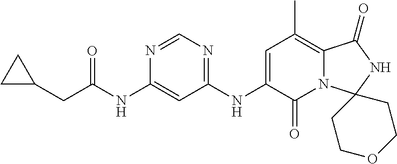 [SMILES: Cc1cc(Nc2cc(NC(=O)CC3CC3)ncn2)c(=O)n2c1C(=O)NC21CCOCC1] |
| 4ET- 03- 022 | 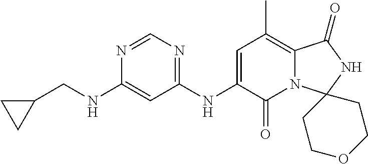 [SMILES: Cc1cc(Nc2cc(NCC3CC3)ncn2)c(=O)n2c1C(=O)NC21CCOCC1] |
| 4ET- 03- 023 | 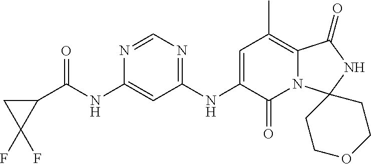 [SMILES: Cc1cc(Nc2cc(NC(=O)C3CC3(F)F)ncn2)c(=O)n2c1C(=O)NC21CCOCC1] |
| 4ET- 03- 024 | 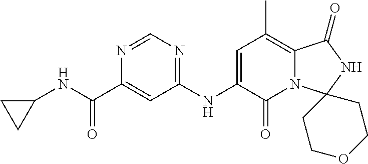 [SMILES: Cc1cc(Nc2cc(C(=O)NC3CC3)ncn2)c(=O)n2c1C(=O)NC21CCOCC1] |
| 4ET- 03- 025 | 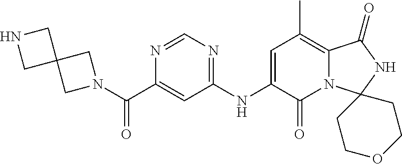 [SMILES: Cc1cc(Nc2cc(C(=O)N3CC4(CNC4)C3)ncn2)c(=O)n2c1C(=O)NC21CCOCC1] |
| 4ET- 03- 026 | 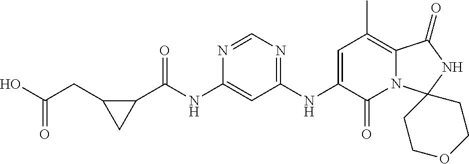 [SMILES: Cc1cc(Nc2cc(NC(=O)C3CC3CC(=O)O)ncn2)c(=O)n2c1C(=O)NC21CCOCC1] |
| 4ET- 03- 027 | 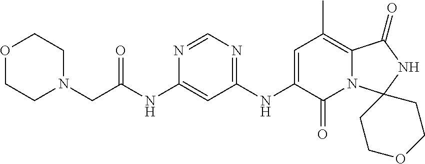 [SMILES: Cc1cc(Nc2cc(NC(=O)CN3CCOCC3)ncn2)c(=O)n2c1C(=O)NC21CCOCC1] |
| 4ET- 03- 028 | 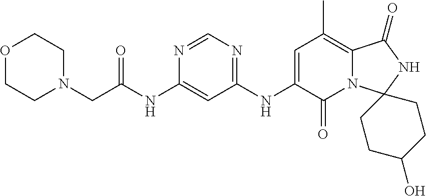 [SMILES: Cc1cc(Nc2cc(NC(=O)CN3CCOCC3)ncn2)c(=O)n2c1C(=O)NC21CCC(O)CC1] |
| 4ET- 03- 029 | 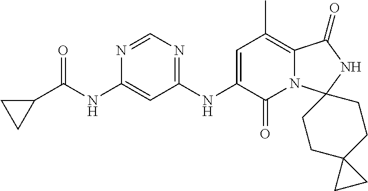 [SMILES: Cc1cc(Nc2cc(NC(=O)C3CC3)ncn2)c(=O)n2c1C(=O)NC21CCC2(CC2)CC1] |
| 4ET- 03- 030 | 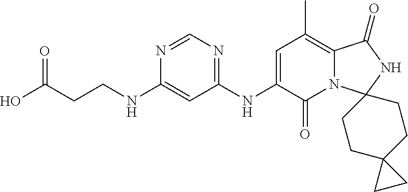 [SMILES: Cc1cc(Nc2cc(NCCC(=O)O)ncn2)c(=O)n2c1C(=O)NC21CCC2(CC2)CC1] |
| 4ET- 03- 031 | 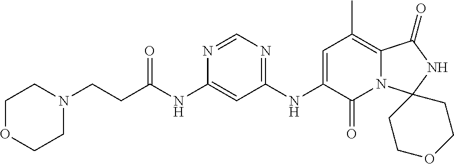 [SMILES: Cc1cc(Nc2cc(NC(=O)CCN3CCOCC3)ncn2)c(=O)n2c1C(=O)NC21CCOCC1] |
| 4ET- 03- 032 | 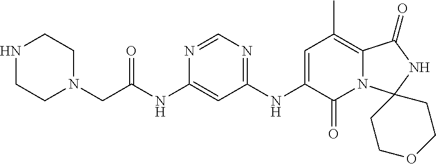 [SMILES: Cc1cc(Nc2cc(NC(=O)CN3CCNCC3)ncn2)c(=O)n2c1C(=O)NC21CCOCC1] |
| 4ET- 03- 033 | 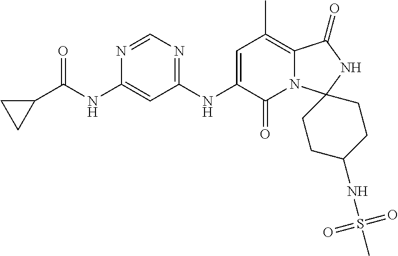 [SMILES: Cc1cc(Nc2cc(NC(=O)C3CC3)ncn2)c(=O)n2c1C(=O)NC21CCC(NS(C)(=O)=O)CC1] |
| 4ET- 03- 034 | 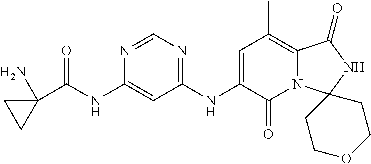 [SMILES: Cc1cc(Nc2cc(NC(=O)C3(N)CC3)ncn2)c(=O)n2c1C(=O)NC21CCOCC1] |
| 4ET- 03- 035 | 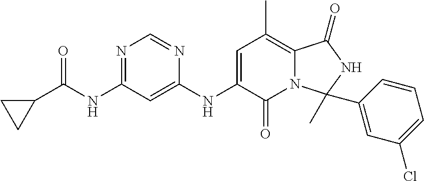 [SMILES: Cc1cc(Nc2cc(NC(=O)C3CC3)ncn2)c(=O)n2c1C(=O)NC2(C)c1cccc(Cl)c1] |
| 4ET- 03- 036 | 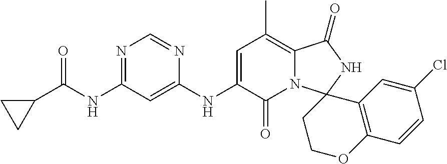 [SMILES: Cc1cc(Nc2cc(NC(=O)C3CC3)ncn2)c(=O)n2c1C(=O)NC21CCOc2ccc(Cl)cc21] |
| 4ET- 03- 039 | 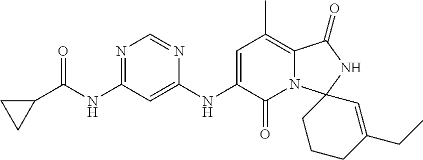 [SMILES: CCC1=CC2(CCC1)NC(=O)c1c(C)cc(Nc3cc(NC(=O)C4CC4)ncn3)c(=O)n12] |
| 4ET- 03- 050A | 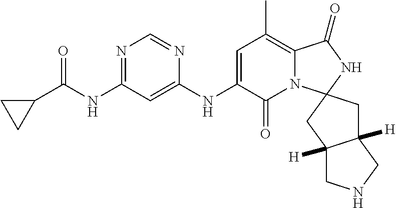 [SMILES: Cc1cc(Nc2cc(NC(=O)C3CC3)ncn2)c(=O)n2c1C(=O)NC21C[C@H]2CNC[C@H]2C1] |
| 4ET- 03- 050B | 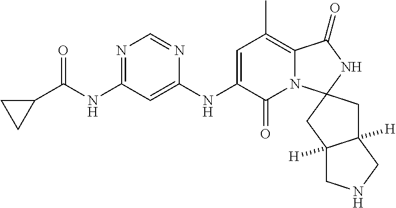 [SMILES: Cc1cc(Nc2cc(NC(=O)C3CC3)ncn2)c(=O)n2c1C(=O)NC21C[C@H]2CNC[C@H]2C1] |
| 4ET- 03- 052A | 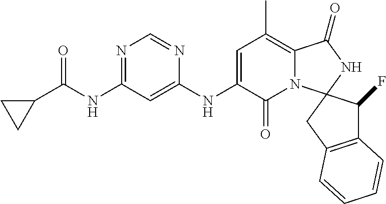 [SMILES: Cc1cc(Nc2cc(NC(=O)C3CC3)ncn2)c(=O)n2c1C(=O)NC21Cc2ccccc2[C@@H]1F] |
| 4ET- 03- 052B | 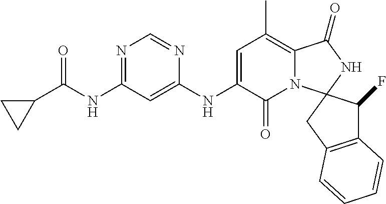 [SMILES: Cc1cc(Nc2cc(NC(=O)C3CC3)ncn2)c(=O)n2c1C(=O)NC21Cc2ccccc2[C@@H]1F] |
| 4ET- 03- 054 | 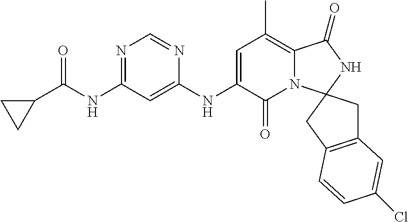 [SMILES: Cc1cc(Nc2cc(NC(=O)C3CC3)ncn2)c(=O)n2c1C(=O)NC21Cc2ccc(Cl)cc2C1] |
| 4ET- 03- 063 | 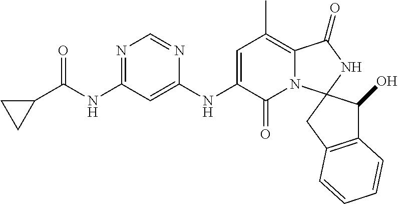 [SMILES: Cc1cc(Nc2cc(NC(=O)C3CC3)ncn2)c(=O)n2c1C(=O)NC21Cc2ccccc2[C@@H]1O] |
| 4ET- 03- 066 | 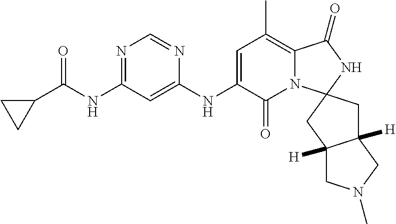 [SMILES: Cc1cc(Nc2cc(NC(=O)C3CC3)ncn2)c(=O)n2c1C(=O)NC21C[C@H]2CN(C)C[C@H]2C1] |
| 4ET- 04- 003 | 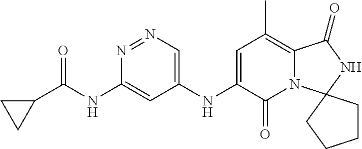 [SMILES: Cc1cc(Nc2cnnc(NC(=O)C3CC3)c2)c(=O)n2c1C(=O)NC21CCCC1] |
| 4ET- 04- 004 | 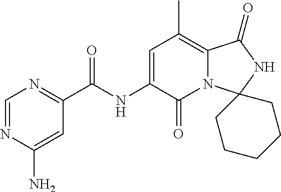 [SMILES: Cc1cc(NC(=O)c2cc(N)ncn2)c(=O)n2c1C(=O)NC21CCCCC1] |
| 4ET- 04- 006 | 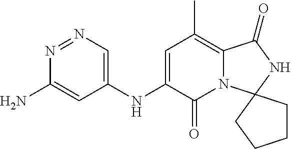 [SMILES: Cc1cc(Nc2cnnc(N)c2)c(=O)n2c1C(=O)NC21CCCC1] |
| 4ET- 04- 010 | 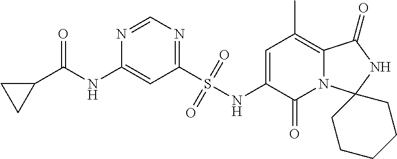 [SMILES: Cc1cc(NS(=O)(=O)c2cc(NC(=O)C3CC3)ncn2)c(=O)n2c1C(=O)NC21CCCCC1] |
| 4ET- 04- 011 | 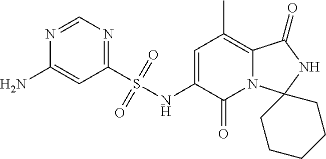 [SMILES: Cc1cc(NS(=O)(=O)c2cc(N)ncn2)c(=O)n2c1C(=O)NC21CCCCC1] |
| 4ET- 04- 012 | 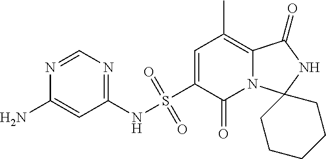 [SMILES: Cc1cc(S(=O)(=O)Nc2cc(N)ncn2)c(=O)n2c1C(=O)NC21CCCCC1] |
| 4ET- 04- 013 | 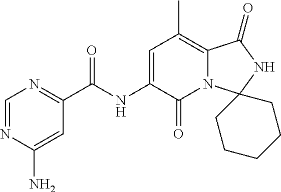 [SMILES: Cc1cc(NC(=O)c2cc(N)ncn2)c(=O)n2c1C(=O)NC21CCCCC1] |
| 4ET- 04- 014 | 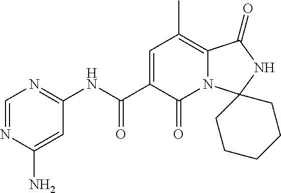 [SMILES: Cc1cc(C(=O)Nc2cc(N)ncn2)c(=O)n2c1C(=O)NC21CCCCC1] |
| 4ET- 04- 015 | 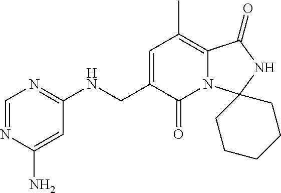 [SMILES: Cc1cc(CNc2cc(N)ncn2)c(=O)n2c1C(=O)NC21CCCCC1] |
| 1 | 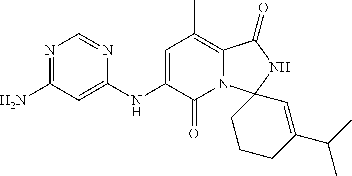 [SMILES: Cc1cc(Nc2cc(N)ncn2)c(=O)n2c1C(=O)NC21C=C(C(C)C)CCC1] |
| 2 | 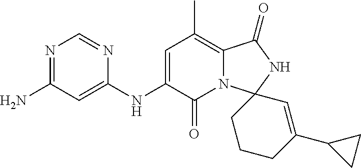 [SMILES: Cc1cc(Nc2cc(N)ncn2)c(=O)n2c1C(=O)NC21C=C(C2CC2)CCC1] |
| 3 | 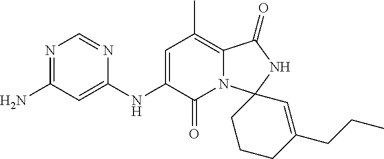 [SMILES: CCCC1=CC2(CCC1)NC(=O)c1c(C)cc(Nc3cc(N)ncn3)c(=O)n12] |
| 4 | 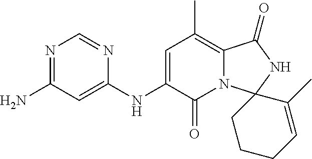 [SMILES: CC1=CCCCC12NC(=O)c1c(C)cc(Nc3cc(N)ncn3)c(=O)n12] |
| 5 | 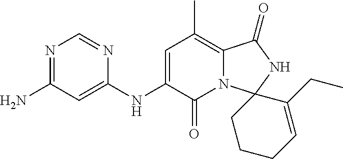 [SMILES: CCC1=CCCCC12NC(=O)c1c(C)cc(Nc3cc(N)ncn3)c(=O)n12] |
| 6 | 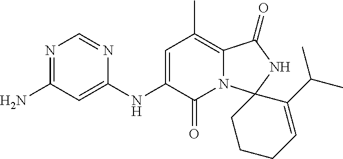 [SMILES: Cc1cc(Nc2cc(N)ncn2)c(=O)n2c1C(=O)NC21CCCC=C1C(C)C] |
| 7 | 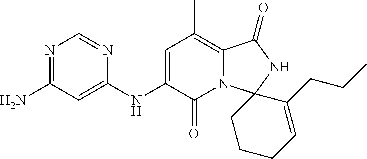 [SMILES: CCCC1=CCCCC12NC(=O)c1c(C)cc(Nc3cc(N)ncn3)c(=O)n12] |
| 8 | 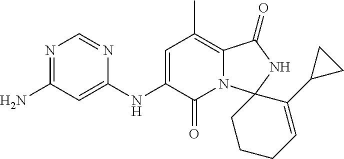 [SMILES: Cc1cc(Nc2cc(N)ncn2)c(=O)n2c1C(=O)NC21CCCC=C1C1CC1] |
| 9 | 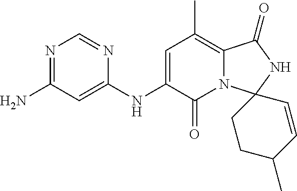 [SMILES: Cc1cc(Nc2cc(N)ncn2)c(=O)n2c1C(=O)NC21C=CC(C)CC1] |
| 10 | 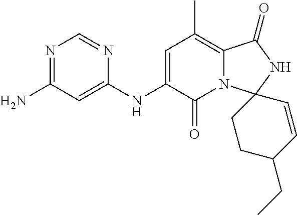 [SMILES: CCC1C=CC2(CC1)NC(=O)c1c(C)cc(Nc3cc(N)ncn3)c(=O)n12] |
| 11 | 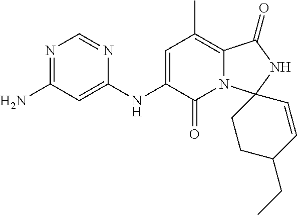 [SMILES: CCC1C=CC2(CC1)NC(=O)c1c(C)cc(Nc3cc(N)ncn3)c(=O)n12] |
| 12 | 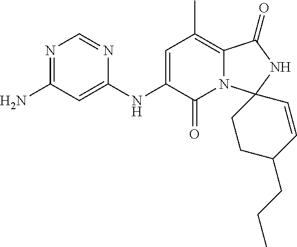 [SMILES: CCCC1C=CC2(CC1)NC(=O)c1c(C)cc(Nc3cc(N)ncn3)c(=O)n12] |
| 13 | 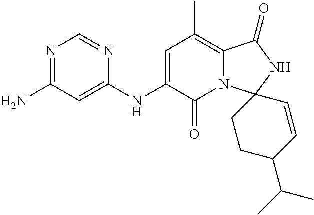 [SMILES: Cc1cc(Nc2cc(N)ncn2)c(=O)n2c1C(=O)NC21C=CC(C(C)C)CC1] |
| 14 | 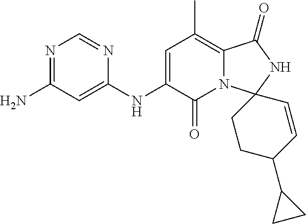 [SMILES: Cc1cc(Nc2cc(N)ncn2)c(=O)n2c1C(=O)NC21C=CC(C2CC2)CC1] |
| 15 | 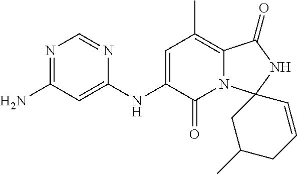 [SMILES: Cc1cc(Nc2cc(N)ncn2)c(=O)n2c1C(=O)NC21C=CCC(C)C1] |
| 16 | 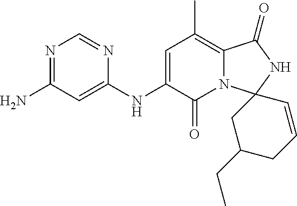 [SMILES: CCC1CC=CC2(C1)NC(=O)c1c(C)cc(Nc3cc(N)ncn3)c(=O)n12] |
| 17 | 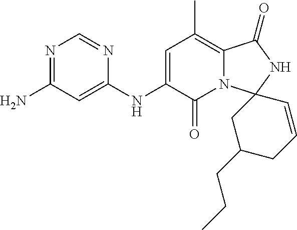 [SMILES: CCCC1CC=CC2(C1)NC(=O)c1c(C)cc(Nc3cc(N)ncn3)c(=O)n12] |
| 18 | 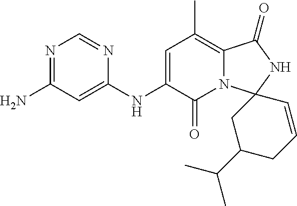 [SMILES: Cc1cc(Nc2cc(N)ncn2)c(=O)n2c1C(=O)NC21C=CCC(C(C)C)C1] |
| 19 | 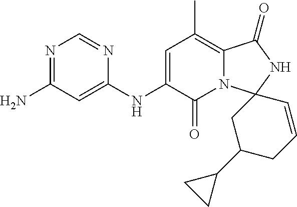 [SMILES: Cc1cc(Nc2cc(N)ncn2)c(=O)n2c1C(=O)NC21C=CCC(C2CC2)C1] |
| 20 | 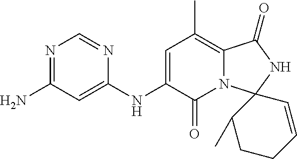 [SMILES: Cc1cc(Nc2cc(N)ncn2)c(=O)n2c1C(=O)NC21C=CCCC1C] |
| 21 | 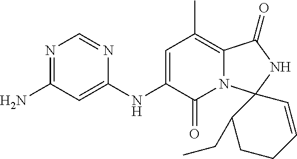 [SMILES: CCC1CCC=CC12NC(=O)c1c(C)cc(Nc3cc(N)ncn3)c(=O)n12] |
| 22 | 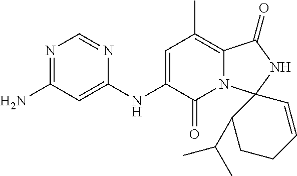 [SMILES: Cc1cc(Nc2cc(N)ncn2)c(=O)n2c1C(=O)NC21C=CCCC1C(C)C] |
| 23 | 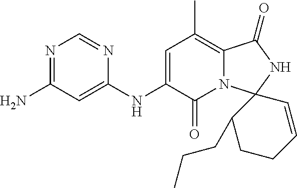 [SMILES: CCCC1CCC=CC12NC(=O)c1c(C)cc(Nc3cc(N)ncn3)c(=O)n12] |
| 24 | 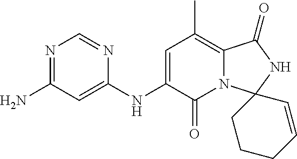 [SMILES: Cc1cc(Nc2cc(N)ncn2)c(=O)n2c1C(=O)NC21C=CCCC1] |
| 25 | 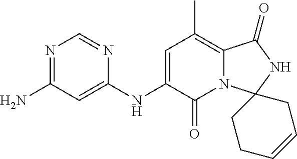 [SMILES: Cc1cc(Nc2cc(N)ncn2)c(=O)n2c1C(=O)NC21CC=CCC1] |
| 26 | 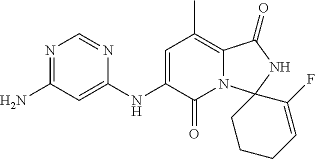 [SMILES: Cc1cc(Nc2cc(N)ncn2)c(=O)n2c1C(=O)NC21CCCC=C1F] |
| 27 | 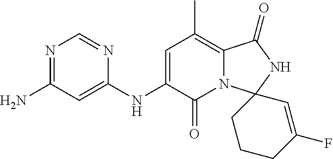 [SMILES: Cc1cc(Nc2cc(N)ncn2)c(=O)n2c1C(=O)NC21C=C(F)CCC1] |
| 28 | 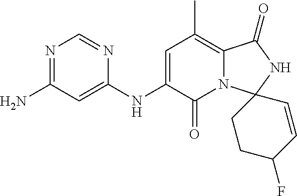 [SMILES: Cc1cc(Nc2cc(N)ncn2)c(=O)n2c1C(=O)NC21C=CC(F)CC1] |
| 29 | 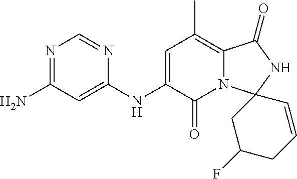 [SMILES: Cc1cc(Nc2cc(N)ncn2)c(=O)n2c1C(=O)NC21C=CCC(F)C1] |
| 30 | 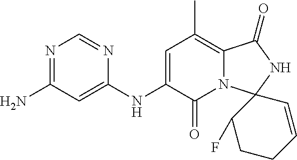 [SMILES: Cc1cc(Nc2cc(N)ncn2)c(=O)n2c1C(=O)NC21C=CCCC1F] |
| 31 | 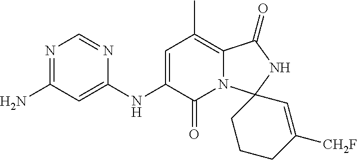 |
| 32 | 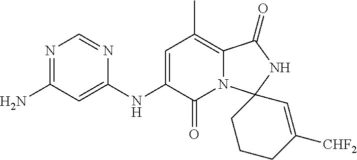 [SMILES: Cc1cc(Nc2cc(N)ncn2)c(=O)n2c1C(=O)NC21C=C(C(F)F)CCC1] |
| 33 | 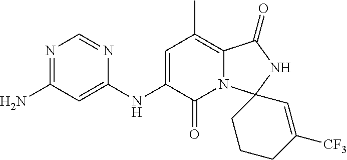 [SMILES: Cc1cc(Nc2cc(N)ncn2)c(=O)n2c1C(=O)NC21C=C(C(F)(F)F)CCC1] |
| 34 | 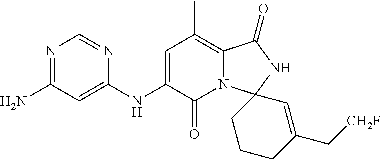 |
| 35 | 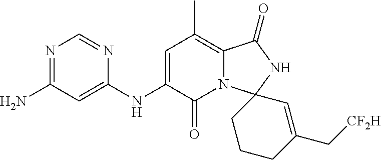 [SMILES: Cc1cc(Nc2cc(N)ncn2)c(=O)n2c1C(=O)NC21C=C(CC(F)F)CCC1] |
| 36 | 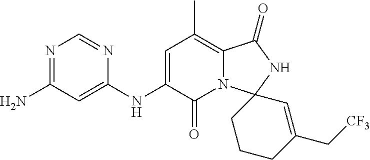 [SMILES: Cc1cc(Nc2cc(N)ncn2)c(=O)n2c1C(=O)NC21C=C(CC(F)(F)F)CCC1] |
| 37 | 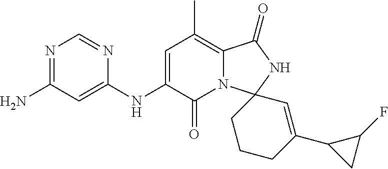 [SMILES: Cc1cc(Nc2cc(N)ncn2)c(=O)n2c1C(=O)NC21C=C(C2CC2F)CCC1] |
| 38 | 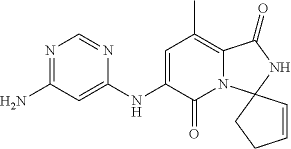 [SMILES: Cc1cc(Nc2cc(N)ncn2)c(=O)n2c1C(=O)NC21C=CCC1] |
| 39 | 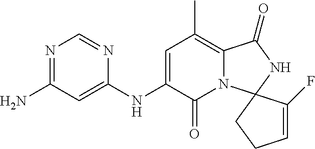 [SMILES: Cc1cc(Nc2cc(N)ncn2)c(=O)n2c1C(=O)NC21CCC=C1F] |
| 40 | 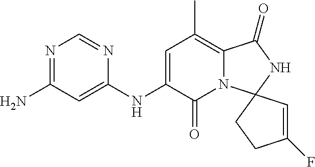 [SMILES: Cc1cc(Nc2cc(N)ncn2)c(=O)n2c1C(=O)NC21C=C(F)CC1] |
| 41 | 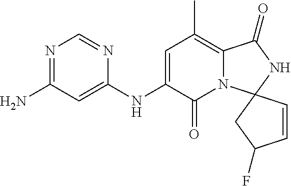 [SMILES: Cc1cc(Nc2cc(N)ncn2)c(=O)n2c1C(=O)NC21C=CC(F)C1] |
| 42 | 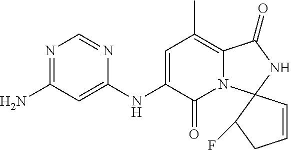 [SMILES: Cc1cc(Nc2cc(N)ncn2)c(=O)n2c1C(=O)NC21C=CCC1F] |
| 43 | 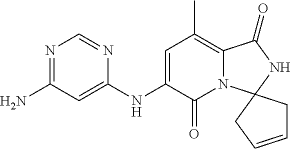 [SMILES: Cc1cc(Nc2cc(N)ncn2)c(=O)n2c1C(=O)NC21CC=CC1] |
| 44 | 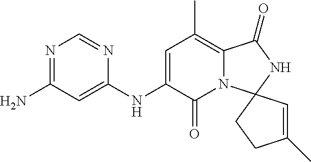 [SMILES: CC1=CC2(CC1)NC(=O)c1c(C)cc(Nc3cc(N)ncn3)c(=O)n12] |
| 45 | 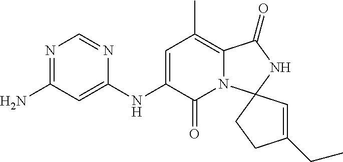 [SMILES: CCC1=CC2(CC1)NC(=O)c1c(C)cc(Nc3cc(N)ncn3)c(=O)n12] |
| 46 | 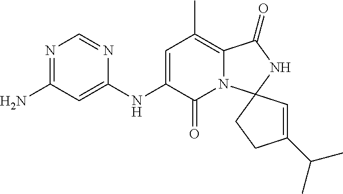 [SMILES: Cc1cc(Nc2cc(N)ncn2)c(=O)n2c1C(=O)NC21C=C(C(C)C)CC1] |
| 47 | 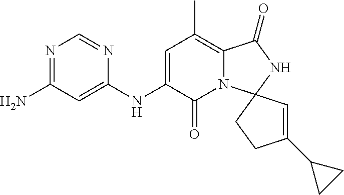 [SMILES: Cc1cc(Nc2cc(N)ncn2)c(=O)n2c1C(=O)NC21C=C(C2CC2)CC1] |
| 48 | 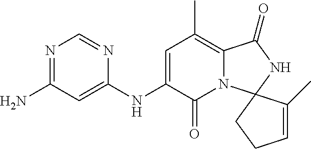 [SMILES: CC1=CCCC12NC(=O)c1c(C)cc(Nc3cc(N)ncn3)c(=O)n12] |
| 49 | 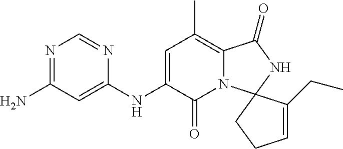 [SMILES: CCC1=CCCC12NC(=O)c1c(C)cc(Nc3cc(N)ncn3)c(=O)n12] |
| 50 | 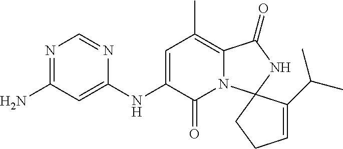 [SMILES: Cc1cc(Nc2cc(N)ncn2)c(=O)n2c1C(=O)NC21CCC=C1C(C)C] |
| 51 | 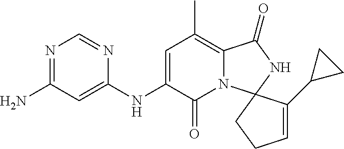 [SMILES: Cc1cc(Nc2cc(N)ncn2)c(=O)n2c1C(=O)NC21CCC=C1C1CC1] |
| 52 | 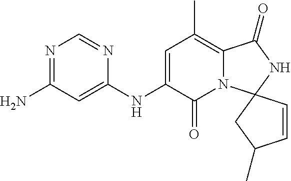 [SMILES: Cc1cc(Nc2cc(N)ncn2)c(=O)n2c1C(=O)NC21C=CC(C)C1] |
| 53 | 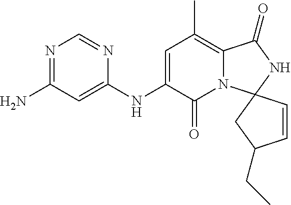 [SMILES: CCC1C=CC2(C1)NC(=O)c1c(C)cc(Nc3cc(N)ncn3)c(=O)n12] |
| 54 | 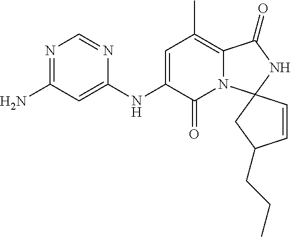 [SMILES: CCCC1C=CC2(C1)NC(=O)c1c(C)cc(Nc3cc(N)ncn3)c(=O)n12] |
| 55 | 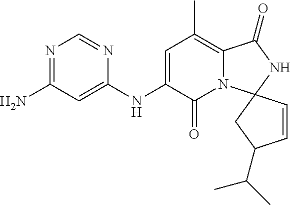 [SMILES: Cc1cc(Nc2cc(N)ncn2)c(=O)n2c1C(=O)NC21C=CC(C(C)C)C1] |
| 56 | 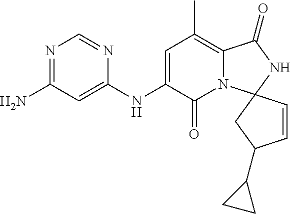 [SMILES: Cc1cc(Nc2cc(N)ncn2)c(=O)n2c1C(=O)NC21C=CC(C2CC2)C1] |
| 57 | 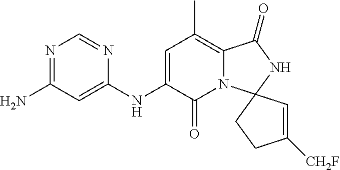 |
| 58 | 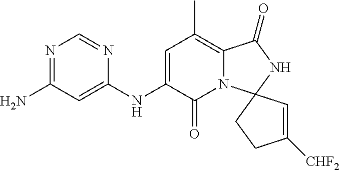 [SMILES: Cc1cc(Nc2cc(N)ncn2)c(=O)n2c1C(=O)NC21C=C(C(F)F)CC1] |
| 59 | 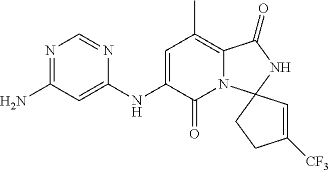 [SMILES: Cc1cc(Nc2cc(N)ncn2)c(=O)n2c1C(=O)NC21C=C(C(F)(F)F)CC1] |
| 60 | 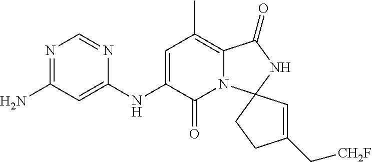 |
| 61 | 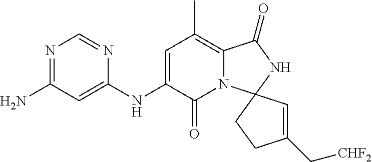 [SMILES: Cc1cc(Nc2cc(N)ncn2)c(=O)n2c1C(=O)NC21C=C(CC(F)F)CC1] |
| 62 | 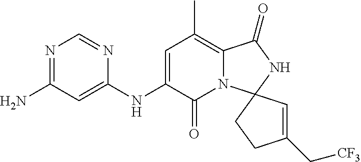 [SMILES: Cc1cc(Nc2cc(N)ncn2)c(=O)n2c1C(=O)NC21C=C(CC(F)(F)F)CC1] |
| 63 | 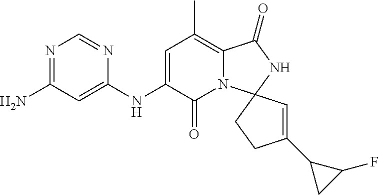 [SMILES: Cc1cc(Nc2cc(N)ncn2)c(=O)n2c1C(=O)NC21C=C(C2CC2F)CC1] |
| 64 | 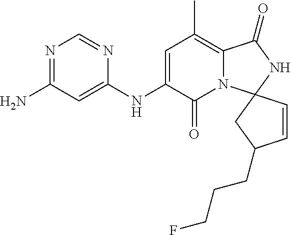 [SMILES: Cc1cc(Nc2cc(N)ncn2)c(=O)n2c1C(=O)NC21C=CC(CCCF)C1] |
| 65 | 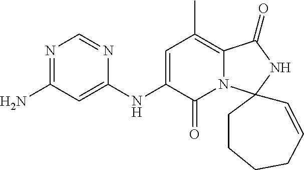 [SMILES: Cc1cc(Nc2cc(N)ncn2)c(=O)n2c1C(=O)NC21C=CCCCC1] |
| 66 | 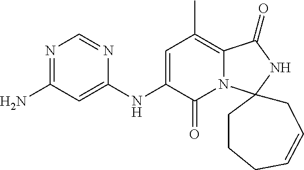 [SMILES: Cc1cc(Nc2cc(N)ncn2)c(=O)n2c1C(=O)NC21CC=CCCC1] |
| 67 | 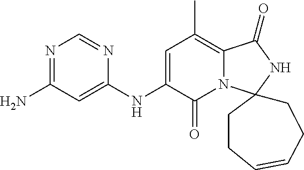 [SMILES: Cc1cc(Nc2cc(N)ncn2)c(=O)n2c1C(=O)NC21CCC=CCC1] |
| 68 | 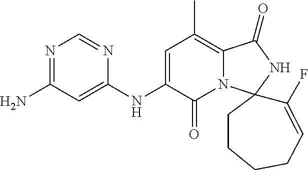 [SMILES: Cc1cc(Nc2cc(N)ncn2)c(=O)n2c1C(=O)NC21CCCCC=C1F] |
| 69 | 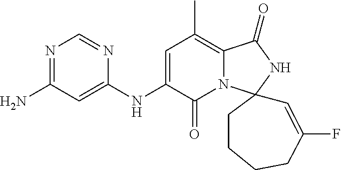 [SMILES: Cc1cc(Nc2cc(N)ncn2)c(=O)n2c1C(=O)NC21C=C(F)CCCC1] |
| 70 | 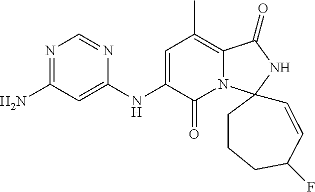 [SMILES: Cc1cc(Nc2cc(N)ncn2)c(=O)n2c1C(=O)NC21C=CC(F)CCC1] |
| 71 | 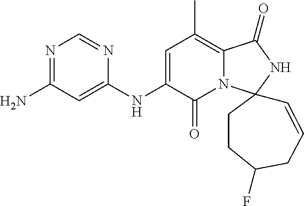 [SMILES: Cc1cc(Nc2cc(N)ncn2)c(=O)n2c1C(=O)NC21C=CCC(F)CC1] |
| 72 | 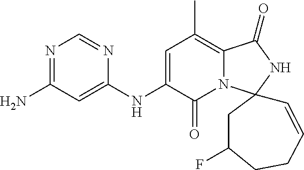 [SMILES: Cc1cc(Nc2cc(N)ncn2)c(=O)n2c1C(=O)NC21C=CCCC(F)C1] |
| 73 | 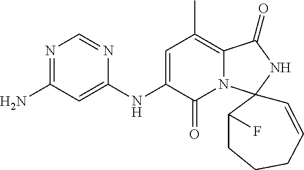 |
| 74 | 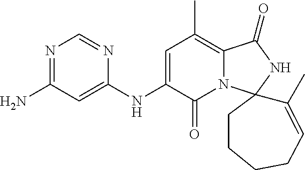 [SMILES: CC1=CCCCCC12NC(=O)c1c(C)cc(Nc3cc(N)ncn3)c(=O)n12] |
| 75 | 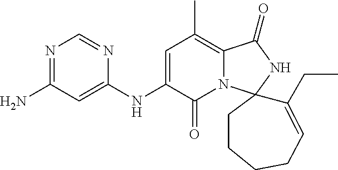 [SMILES: CCC1=CCCCCC12NC(=O)c1c(C)cc(Nc3cc(N)ncn3)c(=O)n12] |
| 76 | 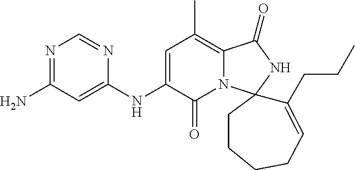 [SMILES: CCCC1=CCCCCC12NC(=O)c1c(C)cc(Nc3cc(N)ncn3)c(=O)n12] |
| 77 | 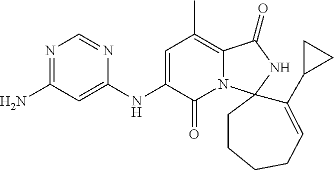 [SMILES: Cc1cc(Nc2cc(N)ncn2)c(=O)n2c1C(=O)NC21CCCCC=C1C1CC1] |
| 78 | 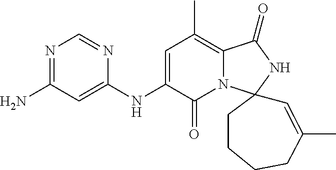 [SMILES: CC1=CC2(CCCC1)NC(=O)c1c(C)cc(Nc3cc(N)ncn3)c(=O)n12] |
| 79 | 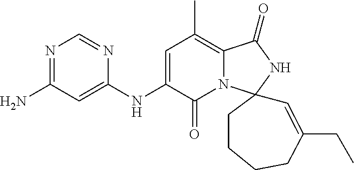 [SMILES: CCC1=CC2(CCCC1)NC(=O)c1c(C)cc(Nc3cc(N)ncn3)c(=O)n12] |
| 80 | 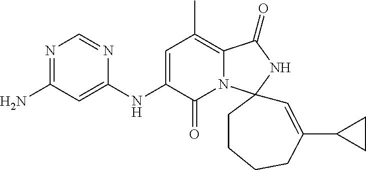 [SMILES: Cc1cc(Nc2cc(N)ncn2)c(=O)n2c1C(=O)NC21C=C(C2CC2)CCCC1] |
| 81 | 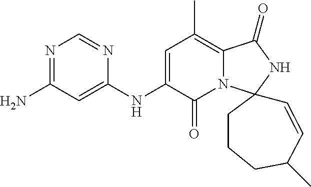 [SMILES: Cc1cc(Nc2cc(N)ncn2)c(=O)n2c1C(=O)NC21C=CC(C)CCC1] |
| 82 | 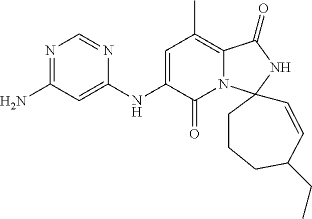 [SMILES: CCC1C=CC2(CCC1)NC(=O)c1c(C)cc(Nc3cc(N)ncn3)c(=O)n12] |
| 83 | 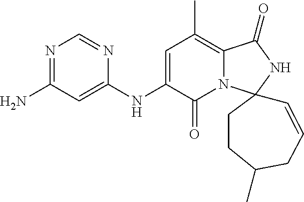 [SMILES: Cc1cc(Nc2cc(N)ncn2)c(=O)n2c1C(=O)NC21C=CCC(C)CC1] |
| 84 | 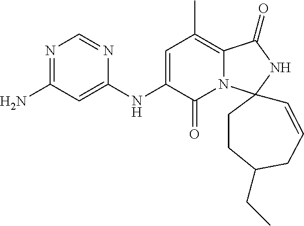 [SMILES: CCC1CC=CC2(CC1)NC(=O)c1c(C)cc(Nc3cc(N)ncn3)c(=O)n12] |
| 85 | 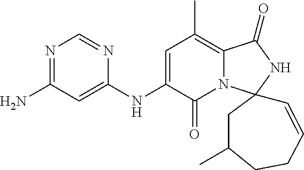 [SMILES: Cc1cc(Nc2cc(N)ncn2)c(=O)n2c1C(=O)NC21C=CCCC(C)C1] |
| 86 | 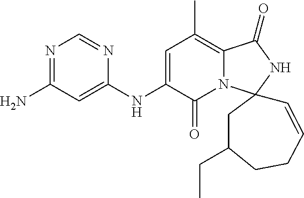 [SMILES: CCC1CCC=CC2(C1)NC(=O)c1c(C)cc(Nc3cc(N)ncn3)c(=O)n12] |
| 87 | 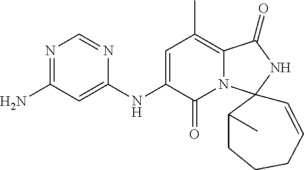 |
| 88 | 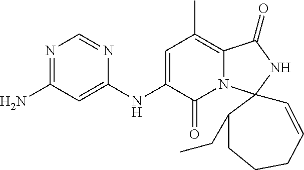 [SMILES: CCC1CCCC=CC12NC(=O)c1c(C)cc(Nc3cc(N)ncn3)c(=O)n12] |
| 89 | 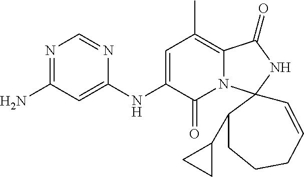 [SMILES: Cc1cc(Nc2cc(N)ncn2)c(=O)n2c1C(=O)NC21C=CCCCC1C1CC1] |
| 90 | 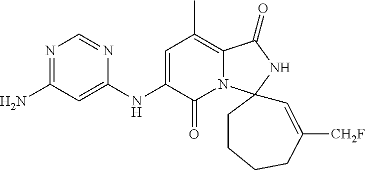 |
| 91 | 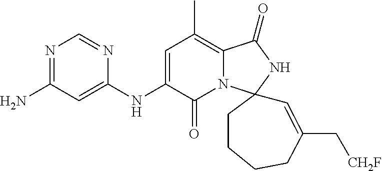 |
| 92 | 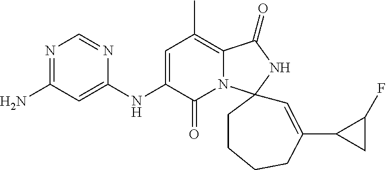 [SMILES: Cc1cc(Nc2cc(N)ncn2)c(=O)n2c1C(=O)NC21C=C(C2CC2F)CCCC1] |

It is understood that in the present description, combinations of substituents and/or variables of the depicted formulae are permissible only if such contributions result in stable compounds.

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

### Pharmaceutical Compositions

To facilitate delivery to a cell, tissue, or patient, an MNK inhibitor of the present disclosure may, in various compositions, be formulated with a pharmaceutically-acceptable carrier, excipient, or diluent. Suitable pharmaceutical carriers, excipients, and/or diluents for use in the present disclosure include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water. Specific formulations of compounds for therapeutic treatment are discussed in Hoover, J. E., Remington's Pharmaceutical Sciences (Easton, Pa.: Mack Publishing Co., 1975) and Liberman and Lachman, eds. Pharmaceutical Dosage Forms (New York, N.Y.: Marcel Decker Publishers, 1980), incorporated by reference herein.

Other embodiments are directed to pharmaceutical compositions. The pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent. Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In treatment methods according to embodiments of the disclosure, an effective amount of at least one compound of Structure (I) or (II) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

Provided herein are pharmaceutical compositions comprising one or more compounds of Structure (I) or (II), and a pharmaceutically acceptable carrier.

Provided herein are pharmaceutical compositions comprising one or more compounds selected from compounds of Structure (I) or (II) and pharmaceutically acceptable diluent(s), excipient(s), and carrier(s). In certain embodiments, the compounds described are administered as pharmaceutical compositions in which one or more compounds selected from compounds of Structure (I) or (II) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I) or (II).

A pharmaceutical composition, as used herein, refers to a mixture of one or more compounds selected from compounds of Structure (I) or (II) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I) or (II) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In addition, an MNK inhibitor as described herein may be formulated with another MNK inhibitor as described herein, another MNK inhibitor, another pain therapeutic, a neuroregeneration therapeutic, or another small molecule or biologic therapeutic, or any combinations thereof. Example pain therapeutics and neuroregeneration thereapeutics are described herein with respect to therapeutic methods using MNK inhibitors.

In one embodiment, one or more compounds selected from compounds of Structure (I) or (II) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I) or (II) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I) or (II) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent or excipient, and one or more compounds selected from compounds of Structure (I) or (II), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I) or (II) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I) or (II). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds selected from compounds of Structure (I) or (II) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I) or (II) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structure (I) or (II) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I) or (II) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### Methods of Treatment

One embodiment of the present disclosure provides a method of treating a MNK-mediated disease or disorder, comprising administering a therapeutically effective amount of a compound of Structure (I) or (II), or the pharmaceutical composition as described herein, to a subject in need thereof.

In more specific embodiments, the disorder is neuropathic pain. In some embodiments, the disease or disorder is Alzheimer's, high fat induced obesity, or viral induced pain.

One embodiment of the present disclosure provides a method of treating a disease or disorder, comprising administering a therapeutically effective amount of a compound of Structure (I) or (II), of the pharmaceutical compositions described herein to a subject in need thereof.

Certain MNK inhbitors of the present disclosure may not pass the blood-brain barrier, which may limit neurological side effects. These periphery-restricted MNK inhibitors may be used for indications where treatment of brain neurons and other tissues is not required. Certain MNK inhbitors of the present disclosure may be able to pass through the blood brain barrier. These brain penetrant MNK inhibitors may be used for any indication where they exhibit a therapeutic effect, but they may be particularly useful for indications, such as Alzheimer's or Huntington's, for administration to neurons or brain tissues.

In certain embodiments, the disease or disorder is Huntington's disease. In some embodiments, the disease is Alzheimer's disease. In some specific embodiments, the disease or disorder is Fragile X syndrome. In some embodiments, the disease or disorder is lupus. In some more specific embodiments, the disease or disorder is viral infection-induced pain. In some embodiments, the disease or disorder is COVID-19 acute respiratory distress syndrome (ARDS). In some specific embodiments, the disease or disorder is non-alcoholic fatty liver disease (NAFLD). In some embodiments, the disease or disorder is high fat diet induced obesity.

Neuropathic pain typically develops over time and may benefit from therapies that interfere with pathways involved in its development and/or continuation.

Some embodiments provide a method for treating neuropathic pain, the method comprising administering a therapeutically effective amount of a compound having the following Structure (II):

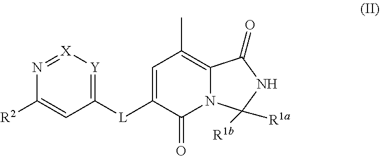

or a pharmaceutically acceptable salt, stereoisomer, tautomer, or prodrug thereof, wherein
R^{1a} is C₁-C₆ alkyl or aryl;
R^{1b} is C₁-C₆ alkyl or aryl,
   or R^{1a} and R^{1b}, together with the carbon to which they are both attached, join to form cycloalkyl, cycloalkenyl, heterocyclyl, aryl, or heteroaryl;
R² is heterocyclyl, —NHR^{3a}, —NHC(═O)R^{3b}, —NHC(═S)R^{3b}, or —C(═O)R^{3c};
R^{3a} is hydrogen, C₁-C₆ alkyl, or C₃-C₆ cycloalkyl, each of which is optionally substituted with one or more substituents selected from the group consisting of hydroxyl, C₃-C₆ cycloalkyl, —NHS(O)₂CH₃, heterocyclyl, —C(═O)OH, —C(═O)N(R^{3d})R^{3d}, or —N(R^{3d})R^{3d};
R^{3b} is C₁-C₆ alkyl, C₃-C₆ cycloalkyl, or heterocyclyl each of which is optionally substituted with one or more substituents selected from the group consisting of hydroxyl, halo, C₁-C₆ alkyl, C₃-C₆ cycloalkyl, —NHS(O)₂CH₃, —N(R^{3d})R^{3d}, heterocyclyl, —C(═O)OH, —C(═O)N(R^{3d})R^{3d}, —NHC(═O)CH₃, —CH₂C(═O)OH,
R^{3c} is —N(R^{3d})R^{3d} or heterocyclyl;
R^{3d} is, at each occurrence, independently hydrogen, C₁-C₆ alkyl, or C₃-C₆ cycloalkyl;
L is —NH— or —CH₂NH—; and
X is N and Y is CH or X is CH and Y is N, to a subject in need thereof.

Some more specific embodiments provide a method for treating neuropathic pain, the method comprising administering a therapeutically effective amount of a compound from Table 1 or a pharmaceutically acceptable salt, stereoisomer, tautomer, or prodrug thereof to a subject in need thereof.

Disease or damage causing neuropathic pain may affect the central nervous system, the peripheral nervous system, or both (as opposed to causes of nociceptive pain, which affect the peripheral nervous system only). Common causes of neuropathic pain include spinal cord injury, multiple sclerosis, central nervous system ischemia, spinal nerve disease, diabetes, other metabolic disorders, herpes zoster infection, HIV-related neuropathies, nutritional deficiencies, toxins, remote manifestations of malignancies, immune mediated disorders, physical trauma to a nerve trunk, such as during surgery, peripheral ischemia, peripheral nerve lesions, nerve compression, chemotherapy or other drug-induced nerve damage, radiation injury, arthritis, autoimmune disease, and infection, typically in an area near the affected nerves.

Neuropathic pain often involves abnormal nociceptor sensitivity. Nociceptors are specialized neurons that detect pain. Nociceptor sensitivity is not fixed; it can change over time. Some causes of neuropathic pain affect nociceptor sensitivity by inducing “peripheral sensitization.” Peripheral sensitization includes spontaneous pathological activity, abnormal excitability, heightened sensitivity to chemical, stimuli, heightened sensitivity to thermal stimuli, heightened sensitivity to mechanical stimuli, and any combinations of these.

Disruption of peripheral sensitization, either by reducing or preventing such peripheral sensitization in the first place or by reducing the degree of already-developed peripheral sensitization, may therefore treat neuropathic pain. Although the disclosure is not limited to one mechanism of action, MNK inhibitors as disclosed herein may disrupt peripheral sensitization.

MNKs phosphorylate the eukaryotic translation initiation factor 4E (eIF4E) and factors that bind to AU-rich elements in the 3′-untranslated region of certain messenger RNAs (mRNAs). MNKs are a subfamily of Ser/Thr kinases, phylogenetically considered Ca²⁺/calmodulin-dependent kinases (CaMKs). MNKs are activated through phosphorylation by the growth factor-stimulated Ras/extracellular signal-regulated kinase pathway and the stress-induced p38 pathway.

Nociceptor sensitization may be blocked by inhibiting activity-dependent mRNA translation through mechanistic targeting of the mitogen-activated protein kinase (MAPK) pathway. The MAPK pathway signal to the eukaryotic translation initiation factor (eIF) 4F complex to regulate the sensitization of nociceptors. MNK inhibitors disclosed herein may interrupt the MAPK pathway, thereby decreasing sensitization of nociceptors and achieving a therapeutic effect on neuropathic pain.

The present disclosure is, therefore, directed to methods of treating neuropathic pain or uses of treatments disclosed herein in treating neuropathic pain by administering an effective amount of an MNK inhibitor disclosed herein.

The present disclosure is also directed to methods of or uses of treatments disclosed herein in inhibiting an eIF4E phosphorylation site in a patient by administering an effective amount of an MNK inhibitor disclosed herein. Such methods may result in treatment of neuropathic pain.

Embodiments of the present disclosure are useful as modulators of neuropathic pain in a host species. The host species or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

Viral infections increase levels of Type 1 Interferons, which are known to interact directly with nociceptors to produce viral induced pain. This pain, in both acute phases of active viral infection or within one or two months after initial viral infection and in long-term or chronic phases at least two months after initial viral infection, may be decreased or alleviated by administering a MNK inhibitor of the present disclosure. In some embodiments, administration for viral induced pain may be similar to that for neuropathic pain.

Lupus, which is characterized by an autoimmune reaction to any of various bodily tissues and organs, is also characterized by excessive amounts of Type 1 Interferons and other inflammatory molecules and may, therefore, also be treated by administering a MNK inhibitor of the present disclosure.

COVID 19 related ARDS is similarly characterized by overproduction of inflammatory molecules that may be decreased by administering a MNK inhibitor of the present disclosure.

Alzheimer's is characterized by intracellular neurofibrillary tanges, extracellular plaques, and increased neuronal cell death, resulting in loss if neurons. Neurofibrillary tangles are typically formed from aggregaged Tau, while extraceullar plaques are typically formed from beta amyloid. Tau found in neurofibrillary tangles is hyperphosophorylated. MNK inhibitors of the present disclosure may disrupt the formation of such tangles and alleviate the symptoms of or slow the progression of Alzheimer's by disrupting Tau hyperphosphorylation. Other diseases and disorders resulting from hyperphosphorylation or inappropriate phosphorylation of Tau may be similarly treated or prevented using MNK inhibitors of the present disclosure.

Huntington's disease is characterized by an incurable breakdown of nerve cells in the brain associated with the presence of CAG repeats in the huntingtin gene. These mutations cause various abnormalities in the ERK pathway that can inappropriately activate MNK. Accordingly, MNK inhibitors of the present disclosure may combat some of the negative effects of MNK activation in Huntington's disease patients and, as a result, alleviave on or more symptoms of the disease or slow the progression of the disease.

High fat induced obesity, also sometimes referred to as diet induced obesity, is associated with phosphorylation of eIF4E. Accordingly, MNK inhibitors of the present disclosure may reduce high fat induced obesity or prevent the development of further obesity.

NAFLD is also associated with obesity and phosphorylation of eIF4E and may be prevented or treated using a MNK inhibitor of the present disclosure.

Fragile X Syndrome results from mutations that trigger epigenetic silencing of the Fmr1 gene. Silencing of Fmr1 results in increased activity of the mitogen-activated protein kinase (MAPK) pathway, including activation of MNK, which phosphorylates eIF4E. Excessive phosphorylation of eIF4E has been directly implicated in the cognitive and behavioral deficits associated with Fragile X Syndrome. Accordingly, a MNK inhibitor of the present disclosure may improve or prevent the development of one or more of the cognitive of behavioral deficits associated with Fragile X Syndrome, particularly if administered early in the patent's life.

Embodiments of the disclosure also relate to the use of compounds according to Structure (I) or (II) and/or physiologically acceptable salts thereof for the prophylactic or therapeutic treatment and/or monitoring of diseases that are caused, mediated and/or modulated by the mitogen-activated proten kinase-interacting kinases (MNK) activity. Furthermore, embodiments of the disclosure relate to the use of compounds according to Structure (I) or (II) and/or physiologically acceptable salts thereof for the production of a medicament for the prophylactic or therapeutic treatment and/or monitoring of diseases. In certain embodiments, the use of a compound according to Structure (I) or (II) or physiologically acceptable salts thereof, for the production of a medicament for the prophylactic or therapeutic treatment.

An MNK inhibitor as disclosed herein may be administered as a single dose or multiple doses. For example, where multiple doses are administered, they may be administered at intervals of 3 times per 24 hours, 2 times per 24 hours, 1 time per 24 hours, 1 time every other day, 1 time every 3 days, 1 time every 4 days, 1 time per week, 2 times per week, or 3 times per week. The MNK inhibitor may also be delivered continuously, for example via a continuous pump. The administration schedule may depend on dose administered, severity of disease, response to treatment, and other factors, or any combinations thereof.

The dose may be any effective amount. However, in specific examples the dose may be 25 mg, 50 mg, 100 mg, 200 mg, or 500 mg.

The initial dose may be greater than subsequent doses or all doses may be the same. The dose may depend on the administration schedule, severity of disease, response to treatment, and other factors, or any combinations thereof. The MNK inhibitor may be administered over a period of one week, two weeks, three weeks, four weeks, one month, two months, three months, four months, five months, six months, seven months, eight months, nine months, ten months, eleven months, one year, two years or three years. The duration of administration may depend on the severity of diseases, response to treatment, and other factors, or any combinations thereof.

For example, a less frequent administration schedule for the same dose may be adopted as the patient responds to treatment. Alternatively, the administration schedule may remain unchanged, but the dose may be decreased as the patient responds to treatment.

As another example, patients who have responded well to treatment and have little or no neuropathic pain or patients being administered the MNK inhibitor as a preventative measure to avoid the development of neuropathic pain may be administered only a low dose of MNK inhibitor and/or have a less frequent administration schedule. Alternatively, patients being administered the MNK inhibitor as a preventative measure to avoid the development of neuropathic pain may be administered a normal or high dose or have a frequent administration schedule but only for a limited duration of time, such as between one and six months, during which neuropathic pain is most likely to develop.

An MNK inhibitor according to the present disclosure may be administered in conjunction with an additional therapeutic, including another MNK inhibitor or a therapeutic that is not an MNK inhibitor, particularly another pain therapeutic, Alzheimer's therapeutic, Huntingon's disease therapeutic, Fragile X Syndrome therapeutic, lupus therapeutic, COVID 19 related ARDS therapeutic, NAFLD therapeutic, or weight loss or other obesity-related therapeutic. Suitable additional therapeutics include both small molecules and biologics. An MNK inhibitor may be administered with any combinations of additional therapeutics.

For example, an MNK inhibitor of the present disclosure may be administered with one or more opioids. Suitable opioids include Morphine, Opium, Hydromorphone, Nicomorphine, Oxycodone, Dihydrocodeine, Diamorphine, Papaveretum, Codeine, Phenylpiperidine derivatives, Ketobemidone, Pethidine, Fentanyl, Pethidine, Diphenylpropylamine derivatives, Piritramide, Dextropropoxyphene, Bezitramide, Methadone, Dextropropoxyphene, Benzomorphan derivatives, Pentazocine, Phenazocine, Oripavine derivatives, Buprenorphine, Etorphine, Oripavine derivatives, Morphinan derivatives, Butorphanol, Nalbuphine, Tilidine, Tramadol and Dezocine, and any combinations thereof.

As another example, an MNK inhibitor of the present disclosure may be administered with one or more gabapentinoids. Suitable gabapentinoids include gabapentin and pregabalin, as well as a gabapentin prodrug, gabapentin enacarbil, and any combinations thereof.

As a further example, an MNK inhibitor of the present disclosure may be administered with one or more other small molecule pain therapeutics. Suitable other small molecule pain therapeutics include salicylates, such as Aspirin (acetylsalicylic acid), Diflunisal and Salsalate, Propionic acid derivatives (Ibuprofen, Dexibuprofen, Naproxen, Fenoprofen, Ketoprofen, Dexketoprofen, Flurbiprofen, Oxaprozin, Loxoprofen), Acetic acid derivatives, (Indomethacin, Tolmetin, Sulindac, Etodolac, Ketorolac, Diclofenac, Nabumetone), Enolic acid (Oxicam) derivatives (Piroxicam, Meloxicam, Tenoxicam, Droxicam, Lornoxicam, Isoxicam), Fenamic acid derivatives or “Fenamates” (Mefenamic acid, Meclofenamic acid, Flufenamic acid, Tolfenamic acid), Selective COX-2 inhibitors (Celecoxib, Rofecoxib, Valdecoxib, Parecoxib, Lumiracoxib, Etoricoxib, Firocoxib), Sulphonanilides such as Nimesulide, and a range of other compounds (Licofelone, Lysine clonixinate, Hyperforin, Figwort), and any combinations thereof.

When administered with another pain therapeutic, an MNK inhibitor according to the present disclosure may allow a reduction in the dose or administration frequency of the other pain therapeutic, or a decrease in the total duration of time the other therapeutic is administered. Such an administration schedule may be particularly beneficial when the additional pain therapeutic is addictive, such as an opioid.

As another example, a MNK inhibitor according to the present disclosure may be administered with an anti-viral therapeutic or an anti-Type 1 Interferon therapeutic, such as Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir,

Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir formerly (Kutapressin), Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tipranavir, Trifluridine, Trizivir, Tromantadine, Truvad, Umifenovir, Valaciclovir (Valtrex), Valganciclovir (Valcyte), Vicriviroc, Vidarabine, Zalcitabine, Zanamivir (Relenza), or Zidovudine.

As another example, a MNK inhibitor of the present disclosure may be administered with an Alzheimer's or other tau-related diseases therapeutic, such as Aducanumab (Aduhelm), Donepezil (Aricept), Rivastigmine (Exelon), Galantamine (Razadyne), Memantine (Namenda), Donepezil and Memantine combination (Namzaric), or Suvorexant (Belsomra).

An yet another example, a MNK inhibitor of the present disclosure may be administered with a weight loss or other obesity-related therapeutic or a NAFLD therapeutic, such as Metformin, Orlistat (Xenical or Alli), Phentermine-topiramate (Qsymia), Naltrexone-Bupropion (Contrave), Liraglutide (Saxenda), Semaglutide (Wegovy), Phentermine, Benzphetamine, Diethylpropion, or Phendimetrazine.

As another example, a MNK inhibitor of the present disclosure may be administered with a Huntington's disease therapeutic, such as Tetrabenazine (Xenazine), Deutetrabenazine (Austedo), Aloperidol (Haldol), Fluphenazine, Risperidone (Risperdal), Olanzapine (Zyprexa), Quetiapine (Seroquel), Amantadine (Gocovri ER, Osmolex ER), Levetiracetam (Keppra, Elepsia XR, Spritam), Clonazepam (Klonopin), Citalopram (Celexa), Escitalopram (Lexapro), Fluoxetine (Prozac, Sarafem), Sertraline (Zoloft), Divalproex (Depakote), Carbamazepine (Carbatrol, Epitol), or Lamotrigine (Lamictal).

As another example, a MNK inhibitor of the present disclosure may be administered with a Fragile X Syndrome therapeutic, such as Sertraline (Zoloft), Metformin, cannabidiols, Acamprosate, Lovastatin, Minocycline, other mood stabilizers, other antianxiety medications, or other antidepressants.

As another example, a MNK inhibitor of the present disclosure may be administered with a COVID 19 ARDS therapeutic, such as an antiviral medicaion, a steroid, an anti-inflammatory medication, or an antibody that specifically binds a SARS-CoV-2 antigen.

The agents disclosed herein or other suitable agents are administered depending on the condition being treated. Hence, in some embodiments the one or more compounds of the disclosure will be co-administered with other agents. When used in combination therapy, the compounds described herein are administered with the second agent simultaneously or separately. This administration in combination can include simultaneous administration of the two or more agents in the same dosage form, simultaneous administration in separate dosage forms, and separate administration. That is, a compound described herein and any additional agent (e.g., an anti-inflammaotry agent, a pain management agent, etc.) can be formulated together in the same dosage form and administered simultaneously. Alternatively, a compound of the disclosure and additional agent can be simultaneously administered, wherein both the agents are present in separate formulations. In another alternative, a compound of the present disclosure can be administered just followed by an additional agent, or vice versa. In some embodiments of the separate administration protocol, a compound of the disclosure and additional agent are administered a few minutes apart, or a few hours apart, or a few days apart. In some embodiments, the compounds of Structure (I) or (II) are administered as a mono-therapy.

The methods of embodiments of embodiments of the disclosure can be performed either in vitro or in vivo. The susceptibility of a particular patient, subject, or cell to treatment with the compounds of Structure (I) or (II) can be particularly determined by in vitro tests, whether in the course of research or clinical application.

### EXAMPLES

The examples and preparations provided below further illustrate and exemplify the compounds of the present disclosure and methods of preparing and testing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art. Methods for producing the compounds described herein is provided below. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

The following General Reaction Schemes illustrate exemplary methods for preparation of compounds of Structure (I) or (II):

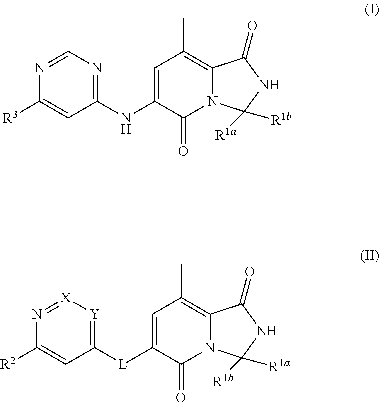

or a pharmaceutically acceptable salt, stereoisomer, tautomer, or prodrug thereof, wherein each of R¹, R^{1b}, R², R³, X, and Y are as defined below.

### General Reaction Scheme 1

The following General Reaction Scheme includes variation of various ingredients in order to achieve different synthetic targets. For example, as referenced below, compounds 6a-6f and compounds 8a-8c have the following structures, respectively:

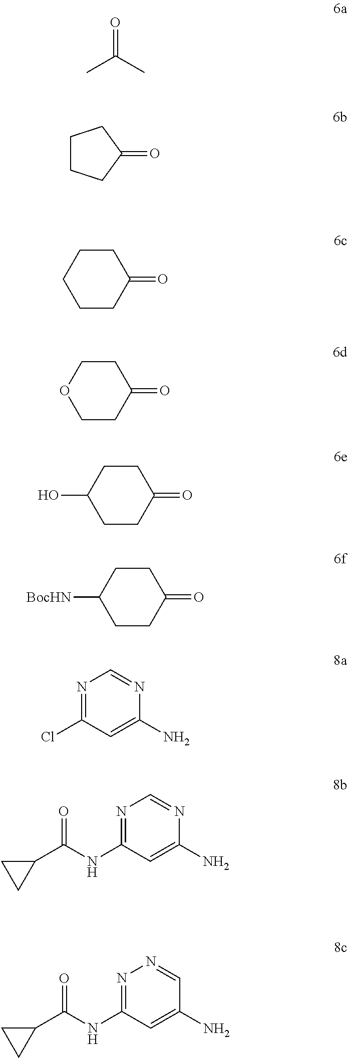

General Reaction Scheme 1 is depicted below:

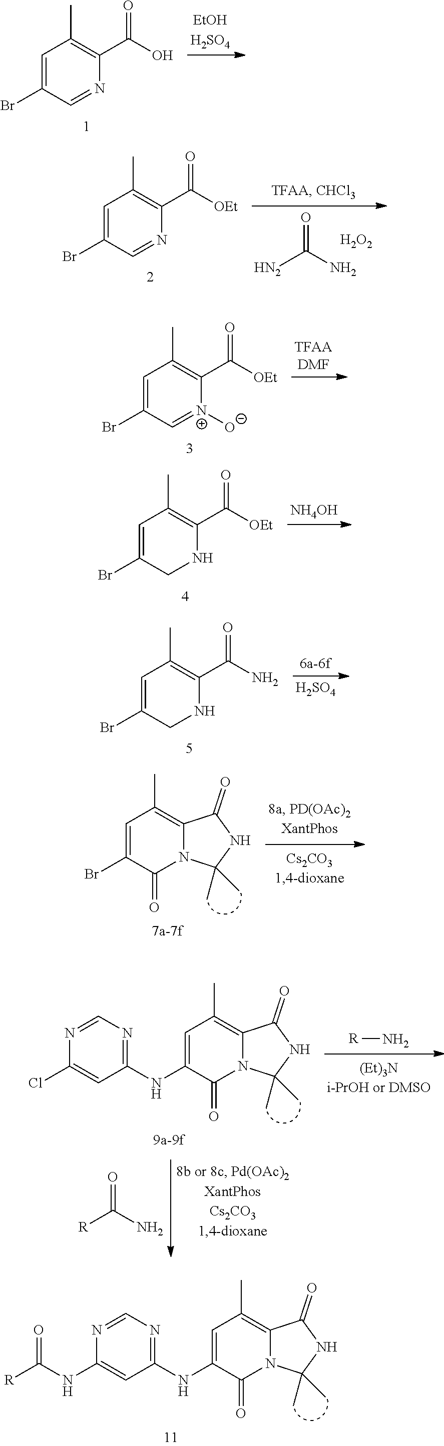

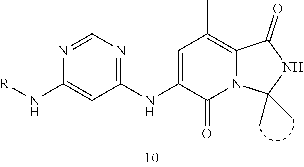

Any of the above reaction schemes can be modified at any step to add and/or modify a substituent or change the order of the steps as appropriate during any stage of the overall synthesis of desired compounds. For example, General Reaction Scheme 1 may be modified after the step yielding compounds 7a-7f according to the following General Reaction Scheme 2, wherein X and Y are either N or C depending on the identity of the reactants used:

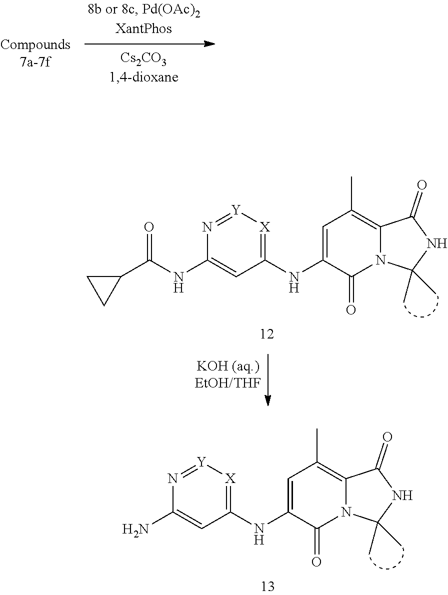

It will also be appreciated by those skilled in the art that in the processes for preparing the compounds described herein the functional groups of intermediate compounds may need to be protected by suitable protecting groups. Such functional groups include, but are not limited to, hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” Prodrugs of compounds of this disclosure are included within the scope of embodiments of the disclosure.

Features of these examples may be combined with elements of the foregoing detailed description unless clearly mutually exclusive. More specific reagent conditions and results from the General Reaction Schemes above are detailed in the Examples below.

The following abbreviations are used in the reaction schemes and synthetic examples herein. This list is not meant to be an all-inclusive list of abbreviations used in the present disclosure as additional standard abbreviations, which are readily understood by those skilled in the art can also be used in the synthetic schemes and examples.
DMA: Dimethylacetamide
DMF: Dimethylformamide
DMSO: Dimethylsulfoxide
TFAA: Trifluoroacetic anhydride
XantPhos: 4,5-Bis(diphenylphosphino)-9,9-dimethylxanthene

### Example 1

### Synthesis of 5-Bromo-3-methyl-6-oxo-1,6-dihydropyridine-2-carboxamide

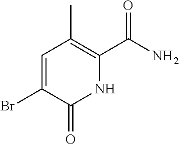

To a solution of compound 1 (10 g, 42.3 mmol) in ethanol (37 mL) was added H₂SO₄ (2.3 mL, 18.4 M, 42.3 mmol) at room temperature. The reaction mixture was heated at 80° C. for 16 h. Solvent was removed under reduced pressure and EtOAc (250 mL) was added. After washing with NaHCO₃ (200 mL×2) and water (200 mL×2), the organic phase was dried over Na₂SO₄, filtered, and concentrated under reduced pressure to afford ethyl 5-bromo-3-methylpicolinate (2, 9.6 g, 39 mmol, 93%) as a colorless liquid. ¹H NMR (400 MHz, CDCl₃) δ 8.58 (s, 1H), 7.76 (s, 1H), 4.43 (q, J=7.1 Hz, 2H), 2.56 (s, 3H), 1.41 (t, J=7.1 Hz, 3H).

To a solution of compound 2 (9.6 g, 39 mmol) in CH₂Cl₂ (111 mL) was added urea hydrogen peroxide (6.4 g, 68.3 mmol), followed by the addition of trifluoroacetic anhydride (9.6 mL, 68.3 mmol) at 0° C. The reaction mixture was stirred at room temperature for 4 h and was poured into ice/water mixture (100 mL). After extraction with CH₂Cl₂ (50 mL×3), the combined organic phase was washed with NaHCO₃ (50 mL×3) and water (50 mL×3), dried over Na₂SO₄, filtered, and concentrated under reduced pressure to afford 5-bromo-2-(ethoxycarbonyl)-3-methylpyridine 1-oxide (3, 10.1 g, 39 mmol, 99%) as a colorless liquid. ¹H NMR (400 MHz, CDCl₃) δ 8.20 (s, 1H), 7.26 (s, 1H), 4.47 (q, J=7.1 Hz, 2H), 2.27 (s, 3H), 1.39 (t, J=7.1 Hz, 3H).

To a solution of compound 3 (10.1 g, 39 mmol) in DMF (30.5 mL) was added trifluoroacetic anhydride (9.6 mL, 68.3 mmol) at 0° C. The reaction mixture was stirred at 40° C. for 8 h and diluted with water (100 mL). After extraction with EtOAc (100 mL×3), the combined organic phase was washed with brine (100 mL×5), dried over Na₂SO₄, filtered, and concentrated under reduced pressure. The residue was purified by Biotage flash chromatography (silica gel, 0% to 30% EtOAc in hexanes) to afford ethyl 5-bromo-3-methyl-6-oxo-1,6-dihydropyridine-2-carboxylate (4, 6.8 g, 26.1 mmol, 67%) as a white solid. ¹H NMR (400 MHz, CDCl₃) δ 7.83 (s, 1H), 4.42 (q, J=7.1 Hz, 2H), 2.45 (s, 3H), 1.41 (t, J=7.1 Hz, 3H).

Ammonium hydroxide (130.5 mL, 28% v/v) was added to compound 4 (6.8 g, 26.1 mmol) at 0° C. The reaction mixture was stirred at 0° C. for 6 h and concentrated under reduced pressure to afford 5-bromo-3-methyl-6-oxo-1,6-dihydropyridine-2-carboxamide (compound 5 of General Reaction Scheme 1, 6.0 g, 26 mmol, 99%) as a white solid. ¹H NMR (400 MHz, DMSO-d₆) δ 7.87 (s, 1H), 7.84 (s, 1H), 7.71 (s, 1H), 2.12 (s, 3H).

### General Procedure A

### Synthesis of 2,3-dihydroimidazo[1,5-a]pyridine-1,5-diones

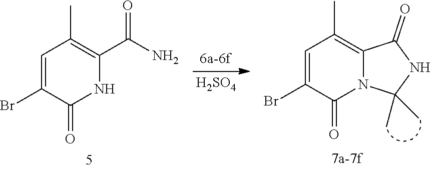

To a solution of compound 5 (1 equiv) in 1,4-dioxane (0.2M) was added ketone 6a-f (4 equiv), followed by the addition of H₂SO₄ (0.5 equiv). Compounds 6a-6f are as depicted in General Reaction Scheme 1.

The reaction mixture was sealed in a pressure vessel and heated in at 100° C. for 16 hours. The reaction mixture was cooled to ambient temperature and concentrated under reduced pressure. The resulting crude material was purified by Biotage flash chromatography (gradient elution, 30→85% EtOAc in hexanes or 0→10% MeOH in CH₂Cl₂) to afford compounds 7a-f.

### Example 2

### Synthesis of 6-Bromo-3,3,8-trimethyl-2,3-dihydroimidazo[1,5-a]pyridine-1,5-dione (7a)

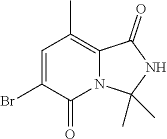

Compound 7d was synthesized according to General Procedure A. Compound 5 (150 mg, 0.65 mmol, 1.0 equiv), acetone (0.48 mL, 6.5 mmol, 10.0 equiv) and H₂SO₄ (3 mg, 0.03 mmol, 0.05 equiv) in 1,4-dioxane (2 mL) generated compound 7a (150 mg, 0.55 mmol, 85% yield) as white solid.

### Example 3

### Synthesis of 6′-Bromo-8′-methyl-2′H-spiro[cyclopentane-1,3′-imidazo[1,5-a]pyridine]-1′,5′-dione (7b)

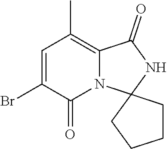 [SMILES: Cc1cc(Br)c(=O)n2c1C(=O)NC21CCCC1]

Compound 7d was synthesized according to General Procedure A. Compound 5 (600 mg, 2.6 mmol), cyclopentanone (6b, 0.92 mL, 10.4 mmol), and H₂SO₄ (0.07 mL, 1.3 mmol) in 1,4-dioxane (5.2 mL) generated compound 7b (585 mg, 1.97 mmol, 76%).

### Example 4

### Synthesis of 6-Bromo-8-methyl-2′H-spiro[cyclohexane-1,3′-imidazo[1,5-a]pyridine]-1′,5′-dione (7c)

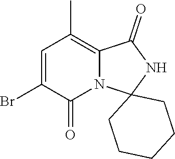 [SMILES: Cc1cc(Br)c(=O)n2c1C(=O)NC21CCCCC1]

Compound 7d was synthesized according to General Procedure A. Compound 5 (1 g, 4.33 mmol), cyclohexanone (6c, 1.8 mL, 17.31 mmol), and H₂SO₄ (0.12 mL, 2.16 mmol) in 1,4-dioxane (16 mL) generated compound 7c (954 mg, 3.06 mmol, 71%).

### Example 5

### Synthesis of 6-Bromo-8-methyl-2′,3′,5′,6′-tetrahydro-2H-spiro[imidazo[1,5-a]pyridine-3,4′-pyran]-1,5-dione (7d)

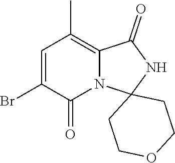 [SMILES: Cc1cc(Br)c(=O)n2c1C(=O)NC21CCOCC1]

Compound 7d was synthesized according to General Procedure A. Compound 5 (500 mg, 2.16 mmol), tetrahydro-4H-pyran-4-one (6d, 0.8 mL, 8.65 mmol), and H₂SO₄ (0.058 mL, 1.08 mmol) in 1,4-dioxane (12 mL) generated compound 7d (486 mg, 1.55 mmol, 72%).

### Example 6

### Synthesis of 6-Bromo-4-hydroxy-8′-methyl-2′H-spiro[cyclohexane-1,3′-imidazo[1,5-a]pyridine]-1′,5′-dione (7e)

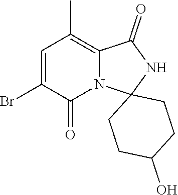 [SMILES: Cc1cc(Br)c(=O)n2c1C(=O)NC21CCC(O)CC1]

Compound 7e was synthesized according to General Procedure A. Compound 5 (300 mg, 1.29 mmol), 4-hydroxycyclohexan-1-one (6e, 592 mg, 5.19 mmol) and H₂SO₄ (0.035 mL, 0.645 mmol) in 1,4-dioxane (13 mL) generated compound 7e (196 mg, 0.60 mmol, 46%).

### Example 7

### Synthesis of N-(6′-bromo-8′-methyl-1′,5′-dioxo-1′,5′-dihydro-2′H-spiro[cyclohexane-1,3′-imidazo[1,5-a]pyridin]-4-yl)methanesulfonamide (7f)

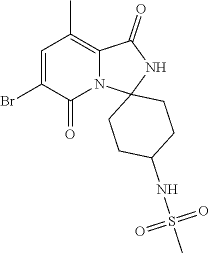 [SMILES: Cc1cc(Br)c(=O)n2c1C(=O)NC21CCC(NS(C)(=O)=O)CC1]

Compound 7e was synthesized according to General Procedure A. Compound 5, tert-butyl (4-oxocyclohexyl)carbamate (6f), and H₂SO₄ in 1,4-dioxane (13 mL) generated intermediate 4-amino-6′-bromo-8′-methyl-2′H-spiro[cyclohexane-1,3′-imidazo[1,5-a]pyridine]-1′,5′-dione. The primary amino intermediate was treated with sulfonyl chloride in CH₂Cl₂ to afford the methylsulfonamide 7f (36% over two steps).

### General Procedure B

### Synthesis of Chloropyrimidinyl Pyridone Intermediates (9a-F)

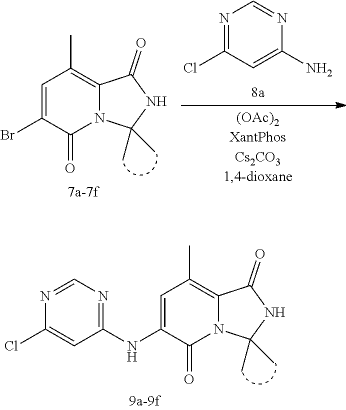 [SMILES: Cc1cc(Br)c(=O)n2c1C(=O)NC2(C)C ; Nc1cc(Cl)ncn1 ; Cc1cc(Nc2cc(Cl)ncn2)c(=O)n2c1C(=O)NC2(C)C]

One of Compound 7a-7f (1 equiv), 4-amino-6-chloropyrimidine 8a (1.2 equiv), Cs₂CO₃ (3 equiv), Xantphos (20 mol %), and Pd(OAc)₂ (10 mol %) was combined in 1,4-dioxane (0.1M) and the mixture was purged with inert gas (nitrogen or argon) for 20 minutes. The reaction vessel was sealed and heated at 90° C. for 16 hours. The reaction mixture was cooled to ambient temperature and washed with water and extracted with 2-propanol/chloroform (v:v/1:4) until full recovery of the product was confirmed (TLC: 80% EtOAc/hexanes). The extracts were combined and concentrated under reduced pressure, and the crude material was purified by either recrystallization (CH₂Cl₂ in hexanes) or Biotage flash chromatography (gradient elution; 0%→10% MeOH in CH₂Cl₂) to afford compounds 9a-f.

### Example 8

### Synthesis of 6-((6-Chloropyrimidin-4-yl)amino)-3,3,8-trimethyl-2,3-dihydroimidazo[1,5-a]pyridine-1,5-dione (9a)

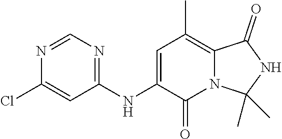 [SMILES: Cc1cc(Nc2cc(Cl)ncn2)c(=O)n2c1C(=O)NC2(C)C]

Compound 9a was synthesized according to General Procedure B; the compound was purified and analyzed according to the following parameters: UHPLC-MS (HESI/APCI): Rₜ 1.36 min, m/z 320.3 [M+H].

### Example 9

### Synthesis of 6′-((6-Chloropyrimidin-4-yl)amino)-8′-methyl-2′H-spiro[cyclopentane-1,3′-imidazo[1,5-a]pyridine]-1′,5′-dione (9b)

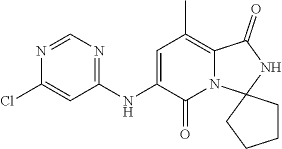 [SMILES: Cc1cc(Nc2cc(Cl)ncn2)c(=O)n2c1C(=O)NC21CCCC1]

Compound 9b was synthesized according to General Procedure B. Compound 7b (270 mg, 1.33 mmol), 4-amino-6-chloropyrimidine 8a (150 mg, 1.6 mmol), Cs2CO3 (950 mg, 4.0 mmol), Xantphos (110 mg, 0.27 mmol), and Pd(OAc)2 (20 mg, 0.13 mmol) in 1,4-dioxane (4.7 mL) generated compound 9b (209 mg, 0.60 mmol, 45%). ¹H NMR (400 MHz, DMSO-d₆) δ 9.88 (s, 1H), 9.62 (s, 1H), 8.59 (s, 1H), 8.50 (s, 1H), 7.45 (s, 1H), 2.76 (m, 2H), 2.40 (s, 3H), 1.94 (m, 2H), 1.80 (m, 2H), 1.67 (m, 2H). UHPLC-MS (HESI/APCI): Rt 1.42 min, m/z 346.2 [M+H].

### Example 10

### Synthesis of 6′-((6-Chloropyrimidin-4-yl)amino)-8′-methyl-2′H-spiro[cyclohexane-1,3′-imidazo[1,5-a]pyridine]-1′,5′-dione (9c)

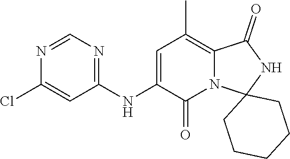 [SMILES: Cc1cc(Nc2cc(Cl)ncn2)c(=O)n2c1C(=O)NC21CCCCC1]

Compound 9c was synthesized according to General Procedure B. Compound 7c (400 mg, 1.29 mmol), 4-amino-6-chloropyrimidine 8a (200 mg, 1.54 mmol), Cs₂CO₃ (1.25 g, 3.9 mmol), Xantphos (149 mg, 0.26 mmol), and Pd(OAc)₂ (29 mg, 0.13 mmol) in 1,4-dioxane (16 mL) generated compound 9c (400 mg, 1.11 mmol, 86%). ¹H NMR (500 MHz, DMSO) δ 8.70 (s, 1H), 8.43 (s, 1H), 8.25 (s, 1H), 6.28 (s, 1H), 5.03 (s, 1H), 3.10 (m, 2H), 2.84-2.76 (m, 2H), 2.42 (s, 3H), 2.04-1.94 (m, 2H), 1.88-1.80 (m, 2H), 1.74-1.66 (m, 2H). UHPLC-MS (HESI/APCI): Rt 1.48 min, m/z 360.2 [M+H].

### Example 11

### Synthesis of 6-((6-Chloropyrimidin-4-yl)amino)-8-methyl-2′,3′,5′,6′-tetrahydro-2H-spiro[imidazo[1,5-a]pyridine-3,4′-pyran]-1,5-dione (9d)

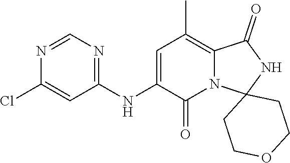 [SMILES: Cc1cc(Nc2cc(Cl)ncn2)c(=O)n2c1C(=O)NC21CCOCC1]

Compound 9d was synthesized according to General Procedure B. The title compound was purified and analyzed according to the following parameters:

UHPLC-MS (HESI/APCI): Rt 1.37 min, m/z 362.2 [M+H].

### General Procedure C

### Synthesis of Aminopyrimidinyl Pyridones (10)

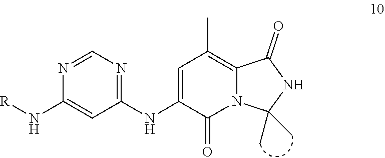

To a mixture of chloropyrimidine 9a-9f (1 equiv) in 2-propanol or DMSO (0.1M) was added triethylamine (5 equiv) and the corresponding amine (5 equiv). The reaction mixture was stirred at temperatures ranging from 100 to 120° C. for 16 hours. The reaction was cooled to ambient temperature and concentrated under reduced pressure. The crude material was purified by Biotage flash chromatography (silica gel, 0%→10% MeOH in CH₂Cl₂; C₁₈, 0% to 10% MeOH in water) and then Prep-TLC if necessary to afford aminopyrimidines of the general structure 10.

### Example 12

### Synthesis of 6′-((6-((2-Hydroxyethyl)amino)pyrimidin-4-yl)amino)-8′-methyl-2′H-spiro[cyclohexane-1,3′-imidazo[1,5-a]pyridine]-1′,5′-dione (4ET-02-001)

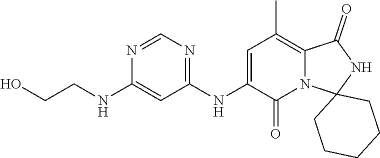 [SMILES: Cc1cc(Nc2cc(NCCO)ncn2)c(=O)n2c1C(=O)NC21CCCCC1]

Compound 4ET-02-001 was synthesized according to General Procedure C. Compound 9c (48 mg, 0.13 mmol), ethanolamine (48 mg, 0.78 mmol), and triethylamine (0.13 mL, 0.93 mmol) in 2-propanol (2 mL) generated compound 4ET-02-001 (10 mg, 0.026 mmol, 20%) as a beige solid. ¹H NMR (400 MHz, DMSO) δ 9.98 (br s, 1H), 8.54 (s, 1H), 8.37 (s, 1H), 8.21 (s, 1H), 6.97 (br s, 1H), 6.26 (s, 1H), 4.70 (t, J=5.4 Hz, 1H), 3.57-3.46 (m, 2H), 3.29-3.23 (m, 2H), 3.00 (dt, J=3.9, 13.0 Hz, 2H), 2.43 (s, 3H), 1.80-1.61 (m, 5H), 1.44 (br d, J=11.5 Hz, 2H), 1.23 (s, 1H). LCMS (ES-API): Rt 3.57 min, m/z 385.2 [M+H].

### Example 13

### Synthesis of 3-((6-((8′-Methyl-1′,5′-dioxo-1′,5′-dihydro-2′H-spiro[cyclohexane-1,3′-imidazo[1,5-a]pyridin]-6′-yl)amino)pyrimidin-4-yl)amino)propanoic acid (4ET-02-006)

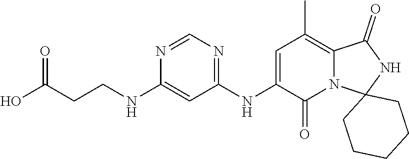 [SMILES: Cc1cc(Nc2cc(NCCC(=O)O)ncn2)c(=O)n2c1C(=O)NC21CCCCC1]

Compound 4ET-02-006 was synthesized according to General Procedure C. Compound 9c (70 mg, 0.19 mmol), beta-alanine (87 mg, 0.97 mmol), and triethylamine (0.16 mL, 1.16 mmol) in DMSO (2.5 mL) generated compound 4ET-02-006 (4 mg, 0.009 mmol, 5%). ¹H NMR (500 MHz, DMSO) δ 10.01 (s, 1H), 8.56 (s, 1H), 8.40 (s, 1H), 8.19 (s, 1H), 7.70 (d, J=13.8 Hz, 1H), 7.07 (s, 1H), 6.24 (s, 1H), 5.03 (s, 2H), 3.21 (d, J=36.3 Hz, 3H), 3.08-2.91 (m, 1H), 2.40 (d, J=32.2 Hz, 1H), 2.18 (s, 1H), 2.04 (s, 1H), 1.70 (dd, J=55.2, 16.1 Hz, 4H), 1.44 (d, J=11.4 Hz, 2H). UHPLC-MS (HESI/APCI): Rt 1.08 min, m/z 413.3 [M+H].

### Example 14

### Synthesis of 8′-Methyl-6′-((6-((2-morpholinoethyl)amino)pyrimidin-4-yl)amino)-2′H-spiro[cyclopentane-1,3′-imidazo[1,5-a]pyridine]-1′,5′-dione (4ET-03-006)

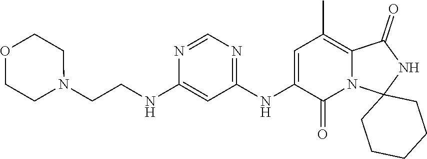 [SMILES: Cc1cc(Nc2cc(NCCN3CCOCC3)ncn2)c(=O)n2c1C(=O)NC21CCCCC1]

Compound 4ET-03-006 was synthesized according to General Procedure C. Compound 9b (40 mg, 0.11 mmol), 2-morpholinoethylamine (72 mg, 0.55 mmol), and triethylamine (56 mg, 0.55 mmol) in 2-propanol (1.1 mL) generated the title compound 4ET-03-006 (5.2 mg, 0.01 mmol, 11%). ¹H NMR (500 MHz, DMSO-d₆) δ 9.82 (s, 1H), 8.68 (s, 1H), 8.44 (s, 1H), 8.22 (s, 1H), 6.93 (s, 1H), 6.28 (s, 1H), 3.58 (s, 4H), 3.32 (s, 2H), 2.80 (m, 2H), 2.48-2.37 (m, 6H), 2.44 (s, 3H), 1.98 (s, 2H), 1.84 (s, 2H), 1.69 (s, 2H); UHPLC-MS (HESI/APCI): Rt 0.90 min, m/z 440.3 [M+H].

### Example 15

### Synthesis of 6′-((6-((2-Hydroxyethyl)amino)pyrimidin-4-yl)amino)-8′-methyl-2′H-spiro[cyclopentane-1,3′-imidazo[1,5-a]pyridine]-1′,5′-dione (4ET-03-007)

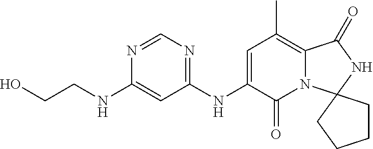 [SMILES: Cc1cc(Nc2cc(NCCO)ncn2)c(=O)n2c1C(=O)NC21CCCC1]

Compound 4ET-03-007 was synthesized according to General Procedure C. Compound 9b (41 mg, 0.12 mmol), ethanolamine (37 mg, 0.6 mmol), and triethylamine (61 mg, 0.6 mmol) in 2-propanol (1.2 mL) generated the title compound 4ET-03-007 (1.1 mg, 0.003 mmol, 2%). 1H NMR (500 MHz, DMSO-d₆) δ 9.82 (s, 1H), 8.64 (s, 1H), 8.42 (s, 1H), 8.22 (s, 1H), 7.02 (s, 1H), 6.28 (s, 1H), 4.74 (s, OH), 3.51 (s, 2H), 3.32 (s, 2H), 2.80 (m, 2H), 2.42 (s, 3H), 1.98 (s, 2H), 1.84 (s, 2H), 1.69 (s, 2H); UHPLC-MS (HESI/APCI): Rt 0.46 min, m/z 371.2 [M+H].

### Example 16

### Synthesis of N-(2-((6-((8′-methyl-1′,5′-dioxo-1′,5′-dihydro-2′H-spiro[cyclopentane-1,3′-imidazo[1,5-a]pyridin]-6′-yl)amino)pyrimidin-4-yl)amino)ethyl)methanesulfonamide (4ET-03-013)

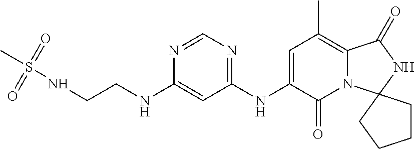 [SMILES: Cc1cc(Nc2cc(NCCNS(C)(=O)=O)ncn2)c(=O)n2c1C(=O)NC21CCCC1]

Compound 4ET-03-013 was synthesized according to General Procedure C. Compound 9b (40 mg, 0.12 mmol), N-(2-aminoethyl)-methanesulfonamide (0.1 mL, 0.6 mmol), and triethylamine (0.1 mL, 0.7 mmol) in 2-propanol (2 mL) generated compound 10e (4ET-03-013) (4 mg, 0.009 mmol, 8%). ¹H NMR (500 MHz, CD3OD) δ 8.37 (s, 1H), 8.26 (s, 1H), 6.14 (s, 1H), 4.63 (s, 3H), 3.66 (s, 1H), 3.54-3.43 (m, 2H), 3.31-3.26 (m, 2H), 3.03 (s, 3H), 2.97 (s, 1H), 2.53 (s, 2H), 2.17-2.08 (m, 2H), 1.95-1.86 (m, 2H), 1.83-1.74 (m, 2H). UHPLC-MS (HESI/APCI): Rt 1.13 min, m/z 448.3 [M+H].

### Example 17

### Synthesis of N-(2-((6-((8′-methyl-1′,5′-dioxo-1′,5′-dihydro-2′H-spiro[cyclohexane-1,3′-imidazo[1,5-a]pyridin]-6′-yl)amino)pyrimidin-4-yl)amino)ethyl)methanesulfonamide (4ET-02-004)

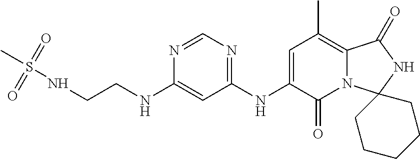 [SMILES: Cc1cc(Nc2cc(NCCNS(C)(=O)=O)ncn2)c(=O)n2c1C(=O)NC21CCCCC1]

Compound 4ET-02-004 was synthesized according to General Procedure C. Compound 9c (70 mg, 0.19 mmol), ethylenediamine (0.07 mL, 0.97 mmol), triethylamine (0.16 mL, 1.17 mmol) in 2-propanol (4 mL) to generate the 1,2-diamine intermediate (20 mg, 0.052 mmol, 27%). Methanesulfonyl chloride (0.004 mL, 0.05 mmol) was added to a mixture of the diamine intermediate (20 mg, 0.05 mmol) and pyridine (5 mg, 0.057 mmol) in CH₂Cl₂ (0.2 mL) at 0° C. The reaction mixture was allowed to warm to room temp (˜23 0° C.), stirred for 16 hours, and then quenched with 3N NaOH. The aqueous layer was extracted with CH₂Cl₂ (3×15 mL), and then a few drops of 12N HCl were added to the aqueous mixture until acidic to pH paper (pH 2-4). The aqueous layer was extracted with 3:1 (CHCl₃/IPA)×3 and combined with the CH₂Cl₂ extracts, dried (Na₂SO₄), filtered, and concentrated under reduced pressure to generate compound 4ET-02-004 (15 mg, 0.032 mmol, 63%). ¹H NMR (500 MHz, CD₃OD) δ 8.42 (s, 1H), 8.03 (s, 1H), 6.35 (s, 1H), 3.56 (s, 1H), 3.31-3.26 (m, 2H), 3.21-3.10 (m, 2H), 2.98 (s, 3H), 2.67-2.58 (m, 3H), 2.54 (s, 3H), 2.0-1.75 (m, 4H), 1.73-1.35 (m, 6H). UHPLC-MS (HESI/APCI): Rt 1.19 min, m/z 462.3 [M+H].

### General Procedure D

### Synthesis of Synthesis of Amidopyrimidinyl Pyridones from Chloropyrimidinyl Pyridones and Amides

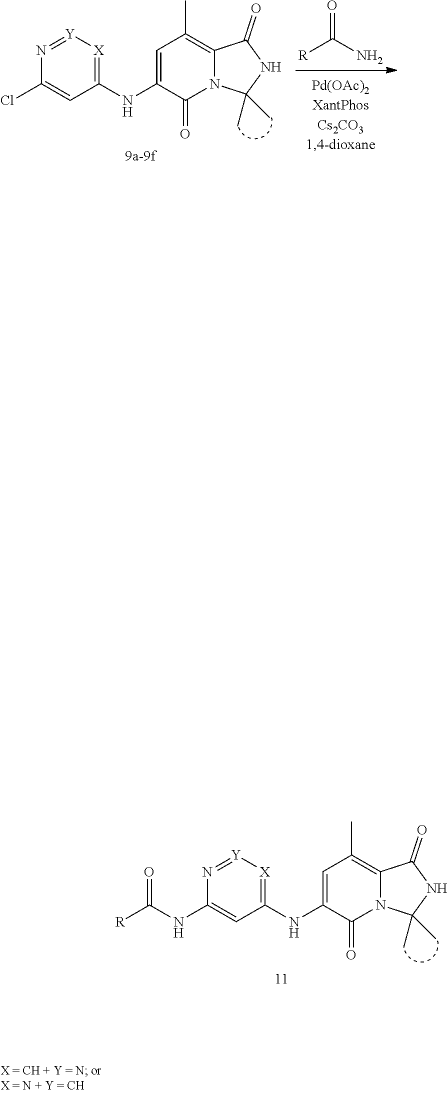

A mixture of the corresponding chloropyrimidine (X═N; Y═CH) pyridone or chloropyridazine (X═CH; Y═N) pyridone (1 equiv), amide (1.2 equiv), Cs₂CO₃ (3 equiv), XantPhos (20 mol %), Pd(OAc)₂ (10 mol %) and 1,4-dioxane (0.1 M) was purged with inert gas (nitrogen or argon) for 20 minutes. The reaction vessel was sealed and heated at 90° C. for 16 hours. The reaction was cooled to ambient temperature and the solvent was removed under reduced pressure. The resulting crude material was purified by Biotage flash chromatography (gradient elution, 0%→10% MeOH in CH₂Cl₂) and then Prep-TLC, if necessary, to afford the amidopyrimidinyl or amidopyridazinyl pyridone aminals (compound 11).

### Example 18

### Synthesis of N-(6-((3,3,8-Trimethyl-1,5-dioxo-1,2,3,5-tetrahydroimidazo[1,5-a]pyridin-6-yl)amino)pyrimidin-4-yl)cyclopropanecarboxamide (4ET-03-002)

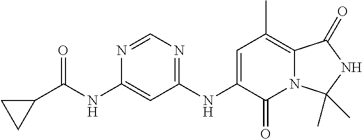 [SMILES: Cc1cc(Nc2cc(NC(=O)C3CC3)ncn2)c(=O)n2c1C(=O)NC2(C)C]

Compound 4ET-03-002 was synthesized according to General Procedure D. Compound 9a (50 mg, 0.16 mmol), cyclopropane carboxamide (16 mg, 0.19 mmol), Cs₂CO₃ (153 mg, 0.47 mmol), Xantphos (18 mg, 0.03 mmol), and Pd(OAc)₂ (4 mg, 0.016 mmol), and 1,4-dioxane (2 mL) generated compound 4ET-03-002 (5.5 mg, 0.015 mmol, 10%).

### Example 19

### Synthesis of N-(6-((8′-Methyl-1′,5′-dioxo-1′,5′-dihydro-2′H-spiro[cyclohexane-1,3′-imidazo[1,5-a]pyridin]-6′-yl)amino)pyrimidin-4-yl)-2-morpholinoacetamide (4ET-02-007)

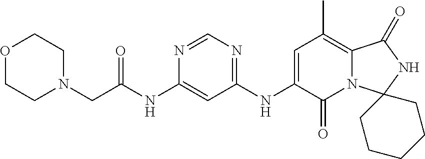 [SMILES: Cc1cc(Nc2cc(NC(=O)CN3CCOCC3)ncn2)c(=O)n2c1C(=O)NC21CCCCC1]

Compound 4ET-02-007 was synthesized according to General Procedure D. Compound 9c (100 mg, 0.28 mmol), 2-morpholinoacetamide (48 mg, 0.33 mmol), Cs₂CO₃ (272 mg, 0.83 mmol), Xantphos (32 mg, 0.06 mmol), and Pd(OAc)₂ (6 mg, 0.03 mmol) in 1,4-dioxane (3 mL) generated compound 4ET-02-007 (13 mg, 0.03 mmol, 10%). ¹H NMR (500 MHz, DMSO) δ 10.07 (d, J=27.4 Hz, 1H), 9.29 (s, 1H), 8.52 (d, J=25.5 Hz, 2H), 7.92 (s, 1H), 3.72-3.57 (m, 3H), 3.27-3.13 (m, 3H), 3.01 (t, J=11.3 Hz, 2H), 2.59-2.52 (m, 2H), 2.44 (d, J=11.5 Hz, 2H), 1.83-1.58 (m, 4H), 1.46 (d, J=11.8 Hz, 2H), 1.24 (s, 2H). UHPLC-MS (HESI/APCI): Rt 1.2 min, m/z 468.3 [M+H].

### Example 20

### Synthesis of N-(6-((8′-Methyl-1′,5′-dioxo-1′,5′-dihydro-2′H-spiro[cyclopentane-1,3′-imidazo[1,5-a]pyridin]-6′-yl)amino)pyrimidin-4-yl)isobutyramide (4ET-03-005)

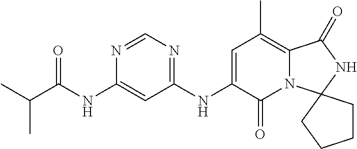 [SMILES: Cc1cc(Nc2cc(NC(=O)C(C)C)ncn2)c(=O)n2c1C(=O)NC21CCCC1]

Compound 4ET-03-005 was synthesized according to General Procedure D. Compound 9b (40 mg, 0.12 mmol), isobutyramide (11 mg, 0.13 mmol), Cs₂CO₃ (117 mg, 0.36 mmol), Xantphos (14 mg, 0.024 mmol), and Pd(OAc)₂ (2.7 mg, 0.012 mmol) in 1,4-dioxane (0.6 mL) generated compound 4ET-03-005 (3.8 mg, 0.01 mmol, 8%). ¹H NMR (500 MHz, DMSO-d₆) δ 10.51 (s, 1H), 9.89 (s, 1H), 9.23 (s, 1H), 8.53 (s, 1H), 8.51 (s, 1H), 7.91 (s, 1H), 2.80 (m, 3H), 2.44 (s, 3H), 1.98 (s, 2H), 1.84 (s, 2H), 1.69 (s, 2H), 1.09 (d, J=6.8 Hz, 6H); UHPLC-MS (HESI/APCI): Rt 1.40 min, m/z 397.3 [M+H].

### General Procedure E

### Synthesis of Amidopyrimidinyl- and Amidopyridazinyl Pyridones from Bromopyridones and Aminopyrimidines/Aminopyridazines

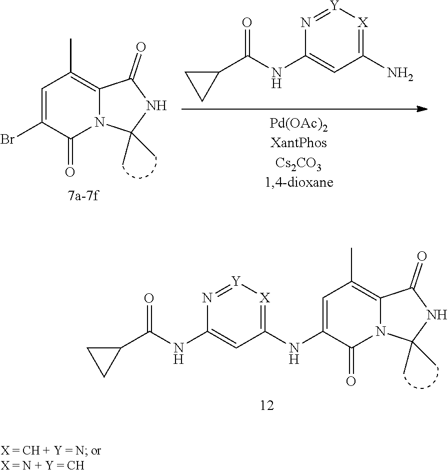

To a mixture of compound 7a-7f (1 equiv), N-(6-aminopyrimidin-4-yl)cyclopropanecarboxamide (8b) or N-(5-aminopyridazin-3-yl)cyclopropanecarboxamide (8c) (1.2 equiv), Cs₂CO₃ (3 equiv), Xantphos (20 mol %), and Pd(OAc)₂ (10 mol %) was added 1,4-dioxane (0.1M), and purged the suspension with inert gas (nitrogen or argon) for 20 minutes. The reaction vessel was sealed and heated at 90° C. for 16 hours and then cooled to ambient temperature. The crude material was purified using Biotage flash chromatography (gradient elution, 0%→10% MeOH in CH₂Cl₂) to afford the amidopyrimidinyl or amidopyridazinyl pyridone aminals (compound 12).

### Example 21

### Synthesis of N-(6-((8′-Methyl-1′,5′-dioxo-1′,5′-dihydro-2′H-spiro[cyclohexane-1,3′-imidazo[1,5-a]pyridin]-6′-yl)amino)pyrimidin-4-yl)cyclopropanecarboxamide (4ET-02-003)

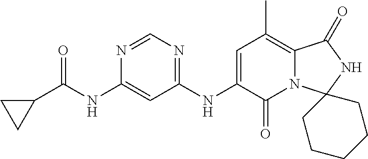 [SMILES: Cc1cc(Nc2cc(NC(=O)C3CC3)ncn2)c(=O)n2c1C(=O)NC21CCCCC1]

Compound 4ET-02-003 was synthesized according to General Procedure E. Compound 7c (149 mg, 0.48 mmol), N-(6-aminopyrimidin-4-yl)cyclopropanecarboxamide (102 mg, 0.575 mmol), Cs₂CO₃ (468 mg, 1.44 mmol), Xantphos (56 mg, 0.096 mmol), and Pd(OAc)₂ (11 mg, 0.048 mmol) in 1,4-dioxane (5 mL) generated the title compound 4ET-02-003 (50 mg, 0.122 mmol, 26%).

### Example 22

### Synthesis of N-(6-((8-Methyl-1,5-dioxo-1,2′,3′,5,5′,6′-hexahydro-2H-spiro[imidazo[1,5-a]pyridine-3,4′-pyran]-6-yl)amino)pyrimidin-4-yl)cyclopropanecarboxamide (4ET-03-009)

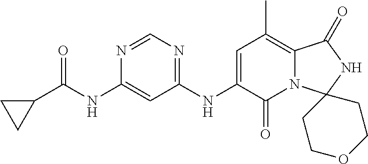 [SMILES: Cc1cc(Nc2cc(NC(=O)C3CC3)ncn2)c(=O)n2c1C(=O)NC21CCOCC1]

Compound 4ET-03-009 was synthesized according to General Procedure E. Compound 7d (100 mg, 0.32 mmol), cyclopropylamido pyrimidinylamide 8b (85 mg, 0.48 mmol), Cs₂CO₃ (312 mg, 0.96 mmol), XantPhos (37 mg, 0.06 mmol), and Pd(OAc)₂ (7.1 mg, 0.03 mmol) in 1,4-dioxane (3.2 mL) generated compound 4ET-03-009 (96 mg, 0.23 mmol, 74%). ¹H NMR (400 MHz, DMSO-d₆) δ 10.86 (s, 1H), 10.32 (s, 1H), 9.18 (s, 1H), 8.53 (s, 1H), 8.50 (s, 1H), 7.89 (s, 1H), 3.93 (m, 2H), 3.69 (m, 2H), 3.25 (m, 2H), 2.45 (s, 3H), 2.02 (pent, J=6.0 Hz, 1H), 1.43 (m, 2H), 0.85 (d, J=6.0 Hz, 4H); UHPLC-MS (HESI/APCI): Rt 0.71 min, m/z 411.3 [M+H].

### Example 23

### Synthesis of N-(6-((4-Hydroxy-8′-methyl-1′,5′-dioxo-1′,5′-dihydro-2′H-spiro[cyclohexane-1,3′-imidazo[1,5-a]pyridin]-6′-yl)amino)pyrimidin-4-yl)cyclopropanecarboxamide (4ET-03-015)

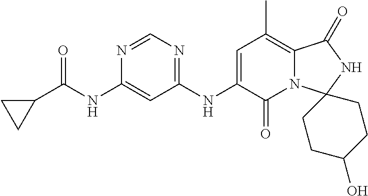 [SMILES: Cc1cc(Nc2cc(NC(=O)C3CC3)ncn2)c(=O)n2c1C(=O)NC21CCC(O)CC1]

Compound 4ET-03-015 was synthesized according to General Procedure E. Compound 7e (100 mg, 0.30 mmol), cyclopropylamido pyrimidinyl amide 8b (82 mg, 0.45 mmol), Cs₂CO₃ (300 mg, 0.92 mmol), Xantphos (35 mg, 0.06 mmol), and Pd(OAc)₂ (6.8 mg, 0.03 mmol), and 1,4-dioxane (3.2 mL) generated compound 4ET-03-015 (99 mg, 0.23 mmol, 78%). ¹H NMR (400 MHz, DMSO-d₆) δ 10.87 (s, 1H), 10.01 (s, 1H), 9.12 (s, 1H), 8.53 (s, 1H), 8.47 (s, 1H), 7.85 (s, 1H), 4.62 (br, 1H), 3.48 (m, 1H), 2.44 (s, 3H), 2.02 (pent, J=6.0 Hz, 1H), 1.87-1.57 (m, 4H), 1.42 (m, 2H), 1.17 (m, 2H), 0.84 (d, J=6.0 Hz, 4H); UHPLC-MS (HESI/APCI): Rt 0.81 min, m/z 425.3 [M+H].

### Example 24

### Synthesis of N-(6-((8′-Methyl-1′,5′-dioxo-1′,5′-dihydro-2′H-spiro[cyclopentane-1,3′-imidazo[1,5-a]pyridin]-6′-yl)amino)pyrimidin-4-yl)cyclopropanecarboxamide (4ET-03-017)

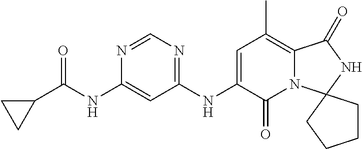 [SMILES: Cc1cc(Nc2cc(NC(=O)C3CC3)ncn2)c(=O)n2c1C(=O)NC21CCCC1]

Compound 4ET-03-017 was synthesized according to General Procedure E. Compound 7b (100 mg, 0.34 mmol), N-(6-aminopyrimidin-4-yl)cyclopropanecarboxamide (8b) (72 mg, 0.4 mmol), Cs₂CO₃ (329 mg, 1.0 mmol), XantPhos (39 mg, 0.07 mmol), and Pd(OAc)₂ (8 mg, 0.034 mmol) in 1,4-dioxane (3.5 mL) generated compound 4ET-03-017 (33 mg, 0.084 mmol, 25%). ¹H NMR (400 MHz, DMSO-d₆) δ 10.83 (s, 1H), 9.84 (s, 1H), 9.16 (s, 1H), 8.50 (s, 1H), 8.46 (s, 1H), 7.83 (s, 1H), 2.80-2.74 (comp, 3H), 2.40 (s, 3H), 2.01-1.94 (m, 2H), 1.82-1.79 (m, 2H), 1.67-1.63 (m, 2H), 0.81 (d, J=4.0 Hz, 4H); UHPLC-MS (HESI/APCI): Rt 1.3 min, m/z 395.3 [M+H].

### Example 25

### Synthesis of N-(5-((8′-Methyl-1′,5′-dioxo-1′,5′-dihydro-2′H-spiro[cyclopentane-1,3′-imidazo[1,5-a]pyridin]-6′-yl)amino)pyridazin-3-yl) cyclopropanecarboxamide (4ET-04-003)

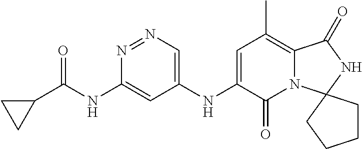 [SMILES: Cc1cc(Nc2cnnc(NC(=O)C3CC3)c2)c(=O)n2c1C(=O)NC21CCCC1]

Compound 4ET-04-003 was synthesized according to General Procedure E. In General Procedure E, Compound 7b (100 mg, 0.33 mmol), N-(5-aminopyridazin-3-yl)cyclopropanecarboxamide (8c) (90 mg, 0.50 mmol), Cs2CO3 (328 mg, 1.01 mmol), XantPhos (39 mg, 0.06 mmol), and Pd(OAc)2 (7.5 mg, 0.03 mmol) in 1,4-dioxane (3.2 mL) generated compound 4ET-04-003 (114 mg, 0.29 mmol, 88%). ¹H NMR (500 MHz, DMSO-d₆) δ 11.15 (s, 1H), 9.93 (s, 1H), 9.04 (s, 1H), 8.88 (s, 1H), 8.12 (s, 1H), 7.33 (s, 1H), 2.79 (m, 2H), 2.40 (s, 3H), 2.04 (pent, J=6.0 Hz, 1H), 1.96 (m, 2H), 1.83 (m, 2H), 1.69 (m, 2H), 0.83 (d, J=6.0 Hz, 4H); UHPLC-MS (HESI/APCI): Rt 1.17 min, m/z 395.3 [M+H].

### General Procedures F1 and F2

### Synthesis of Synthesis of Aminopyrimidinyl- and Aminopyridazinyl Pyridones from Cyclopropylamides

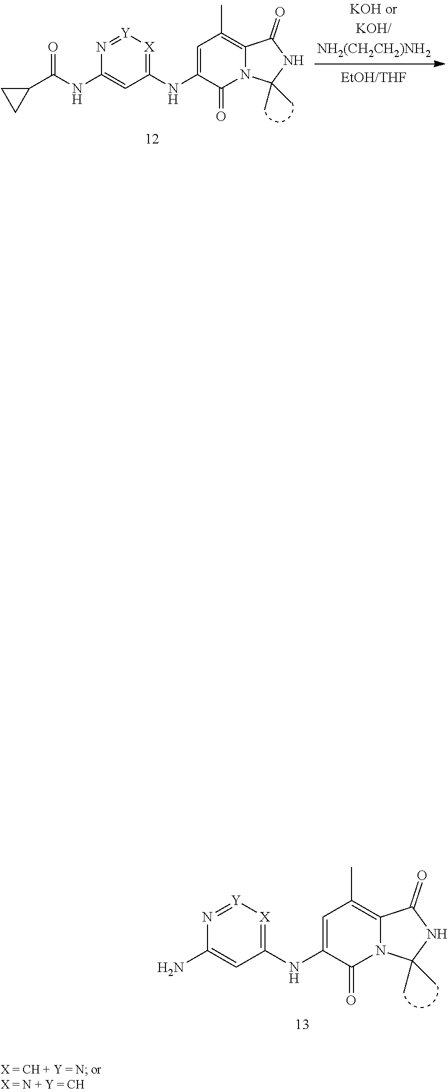

General Procedure F1: Aqueous potassium hydroxide (12 equiv) and ethylenediamine (12 equiv) were sequentially added to a solution of cyclopropylamide 12 (1 equiv) in tetrahydrofuran and ethanol (1:1, v/v). After stirring at room temperature for 24 hours, the mixture was concentrated under reduced pressure, and the residue was diluted with dichloromethane and then washed with water. The organic layer was dried (Na₂SO₄), filtered, and concentrated under reduced pressure. The residue was purified on an InterChim automated chromatography system (silica gel column), eluting with a gradient of 0 to 10% methanol in dichloromethane to give the aminopyrimidine or aminopyridazine.

General Procedure F2: To a mixture of cyclopropylamide (1 equiv) in EtOH/THF/water (v:v:v/2:1:1) was added KOH (6M in H₂O; 4.8 equiv). The resulting mixture was stirred at ambient temperature for 16 hours, after which the mixture was concentrated under reduced pressure and azeotropically washed with toluene. The crude material was purified by Biotage flash chromatography (gradient elution; 0%→25% MeOH in CH₂Cl₂) to afford the aminopyrimidine or aminopyridazine.

### Example 26

### Synthesis of 6′-((6-Aminopyrimidin-4-yl)amino)-8′-methyl-2′H-spiro[cyclopentane-1,3′-imidazo[1,5-a]pyridine]-1′,5′-dione (4ET-01-001)

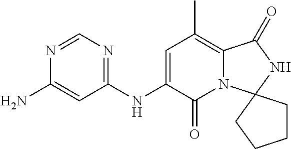 [SMILES: Cc1cc(Nc2cc(N)ncn2)c(=O)n2c1C(=O)NC21CCCC1]

Compound 4ET-01-001 was synthesized according to General Procedure F1. Potassium hydroxide (214 mg, 3.82 mmol, 12.0 equiv) in water (1 mL), ethylenediamine (230 mg, 3.82 mmol, 12.0 equiv), and compound 4ET-03-017 (125 mg, 0.318 mmol, 1.0 equiv) in tetrahydrofuran (2 mL) and ethanol (2 mL), to give compound 4ET-01-001 (12 mg, 12% yield) as an off-white solid. ¹H NMR (400 MHz, DMSO-d₆) δ 9.78 (s, 1H), 8.62 (s, 1H), 8.40 (s, 1H), 8.17 (s, 1H), 6.49 (s, 2H), 6.16 (d, J=0.9 Hz, 1H), 2.84-2.75 (m, 2H), 2.41 (s, 3H), 2.02-1.93 (m, 2H), 1.88-1.78 (m, 2H), 1.68 (td, J=5.8, 11.9 Hz, 2H); ¹³C NMR (100 MHz, DMSO-d₆) δ 164.4, 161.6, 159.9, 158.1, 153.8, 134.0, 122.3, 121.3, 117.0, 88.2, 36.0, 25.3, 14.2; LCMS (ES-API): Rt 3.0 min, m/z 327.1 [M+H].

### Example 27

### Synthesis of 6-((6-Aminopyrimidin-4-yl)amino)-8-methyl-2′,3′,5′,6′-tetrahydro-2H-spiro[imidazo[1,5-a]pyridine-3,4′-pyran]-1,5-dione (4ET-01-002)

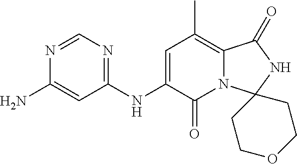 [SMILES: Cc1cc(Nc2cc(N)ncn2)c(=O)n2c1C(=O)NC21CCOCC1]

Compound 4ET-01-002 was synthesized according to General Procedure F1. Potassium hydroxide (51 mg, 0.91 mmol, 12.0 equiv) in water (0.5 mL), ethylenediamine (54 mg, 0.91 mmol, 12.0 equiv), and compound 4ET-03-009 (31 mg, 0.076 mmol, 1.0 equiv) in tetrahydrofuran (0.7 mL) and ethanol (0.7 mL), to give compound 4ET-01-002 (23 mg, 89% yield) as a white solid. ¹H NMR (400 MHz, DMSO-d₆) δ 10.2 (br s, 1H), 8.60 (s, 1H), 8.41 (s, 1H), 8.17 (s, 1H), 6.50 (s, 2H), 6.19 (d, J=0.7 Hz, 1H), 3.95-3.90 (m, 2H), 3.73-3.66 (m, 2H), 3.26 (dt, J=5.5, 13.0 Hz, 2H), 2.43 (s, 3H), 1.42 (br d, J=12.5 Hz, 2H); ¹³C NMR (100 MHz, DMSO-d₆) δ 164.4, 162.3, 159.8, 158.1, 154.1, 134.3, 121.9, 121.8, 117.5, 88.3, 77.0, 63.8, 33.3, 14.3; LCMS (ES-API): Rt 2.6 min, m/z 343.1 [M+H].

### Example 28

### Synthesis of 6-((6-Aminopyrimidin-4-yl)amino)-3,3,8-trimethyl-2,3-dihydroimidazo[1,5-a]pyridine-1,5-dione (4ET-01-003)

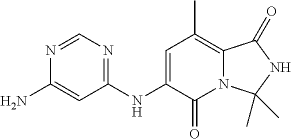 [SMILES: Cc1cc(Nc2cc(N)ncn2)c(=O)n2c1C(=O)NC2(C)C]

Compound 4ET-01-003 was synthesized according to General Procedure F1. Potassium hydroxide (84 mg, 1.5 mmol, 12.0 equiv) in water (0.5 mL), ethylenediamine (90 mg, 1.5 mmol, 12.0 equiv), and compound 4ET-03-002 (46 mg, 0.12 mmol, 1.0 equiv) in tetrahydrofuran (1.0 mL) and ethanol (1.0 mL), to give compound 4ET-01-003 (33 mg, 88% yield) as a white solid. ¹H NMR (400 MHz, DMSO-d₆) δ 9.44 (s, 1H), 8.58 (s, 1H), 8.38 (s, 1H), 8.17 (s, 1H), 6.49 (s, 2H), 6.17 (d, J=0.9 Hz, 1H), 2.41 (s, 3H), 1.78 (s, 6H); ¹³C NMR (100 MHz, DMSO-d₆) δ 164.4, 161.5,159.9, 158.1, 153.9, 134.0, 122.0, 121.9, 117.1, 88.2, 76.3, 25.3, 14.3; LCMS (ES-API): Rt 2.6 min, m/z 301.1 [M+H].

### Example 29

### Synthesis of N-(6′-((6-Aminopyrimidin-4-yl)amino)-8′-methyl-1′,5′-dioxo-1′,5′-dihydro-2′H-spiro[cyclohexane-1,3′-imidazo[1,5-a]pyridin]-4-yl)methanesulfonamide (4ET-01-004)

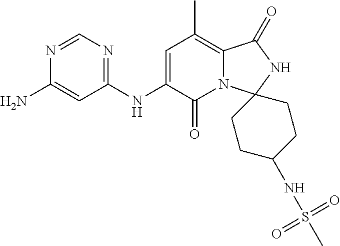 [SMILES: Cc1cc(Nc2cc(N)ncn2)c(=O)n2c1C(=O)NC21CCC(NS(C)(=O)=O)CC1]

Compound 4ET-01-004 was synthesized according to General Procedure F1. Potassium hydroxide (48 mg, 0.86 mmol, 12.0 equiv) in water (0.35 mL), ethylenediamine (52 mg, 0.86 mmol, 12.0 equiv) and compound 4ET-03-033 (vida infra) (36 mg, 0.07 mmol, 1.0 equiv) in tetrahydrofuran (0.7 mL) and ethanol (0.7 mL) to give 20 mg, which was repurified to give 6 mg compound 4ET-01-004 as an off-white solid (19%). ¹H NMR (400 MHz, DMSO-d₆) δ 8.47 (s, 1H), 8.33 (s, 1H), 8.13 (s, 1H), 7.30 (br s, 2H), 6.49 (s, 2H), 6.13 (s, 1H), 3.14-3.10 (m, 1H), 2.93 (s, 3H), 2.39 (s, 3H), 1.96-1.91 (m, 2H), 1.73-1.64 (m, 2H), 1.45 (br d, J=16.0 Hz, 2H), 1.26-1.14 (m, 2H); UHPLC-MS (HESI/APCI): Rt 0.78 min, m/z 434.2 [M+H].

### Example 30

### Synthesis of 6′-((6-Aminopyrimidin-4-yl)amino)-4-hydroxy-8′-methyl-2′H-spiro[cyclohexane-1,3′-imidazo[1,5-a]pyridine]-1′,5′-dione (4ET-01-005)

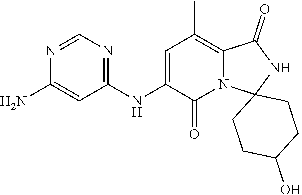 [SMILES: Cc1cc(Nc2cc(N)ncn2)c(=O)n2c1C(=O)NC21CCC(O)CC1]

Compound 4ET-01-005 was synthesized according to General Procedure F2. cyclopropylamide 11f (4ET-03-015) (50 mg, 0.12 mmol) and KOH (6M in H₂O; 0.1 mL, 0.58 mmol) in EtOH/THF/water (v:v:v/2:1:1, 2.0 mL) to give compound 4ET-01-005 (25 mg, 0.07 mmol, 63%). ¹H NMR (400 MHz, DMSO-d₆) δ 10.00 (s, 1H), 8.56 (s, 1H), 8.37 (s, 1H), 8.17 (s, 1H), 6.51 (br, 2H), 6.15 (s, 1H), 4.62 (br. 1H), 3.57-3.40 (m, 2H), 3.10 (m, 1H), 2.42 (s, 3H), 1.87-1.56 (m, 4H), 1.30 (m, 2H); UHPLC-MS (HESI/APCI): Rt 0.47 min, m/z 357.3 [M+H].

Others of compounds 4ET-01-001-005, 4ET-02-001-023, 4ET-03-001-034, 4ET-04-003 for which the synthesis is not specifically set forth in these examples may be readily synthesized by applying general principles known to one of ordinary skill in the art to the synthesis methods described herein.

### Example 31

### Synthesis of 6′-Bromo-3,8′-dimethyl-2′H-spiro[cyclohexane-1,3′-imidazo[1,5-a]pyridin]-2-ene-1′,5′-dione

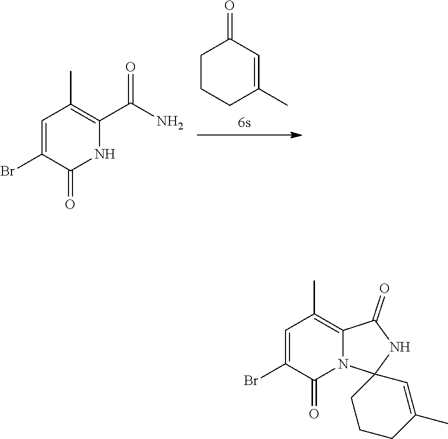

In general procedure A, compound 5 (100 mg, 0.433 mmol), 3-methylcyclohex-2-en-1-one (6s, 477 mg, 4.328 mmol), H₂SO₄ (0.012 mL, 0.216 mmol), and 1,4-dioxane (4.0 mL) generated the title compound (60 mg, 0.186 mmol, 43%). ¹H NMR (400 MHz, CDCl₃) δ 7.75 (s, 1H), 6.70 (s, 1H), 5.51 (s, 1H), 3.89 (d, J=16.9 Hz, 1H), 3.21 (td, J=12.5, 6.9 Hz, 1H), 2.48 (s, 3H), 2.24 (m, 2H), 1.78 (d, J=16.6 Hz, 1H), 1.70 (s, 3H), 1.59 (d, J=6.6 Hz, 1H). UHPLC-MS (ESI): Rt 1.05 min, m/z 323.2 [M]⁺.

### Example 32

### Synthesis of N-(6-((3,8′-Dimethyl-1′,5′-dioxo-1′,5′-dihydro-2′H-spiro[cyclohexane-1,3′-imidazo[1,5-a]pyridin]-2-en-6′-yl)amino)pyrimidin-4-yl)cyclopropanecarboxamide

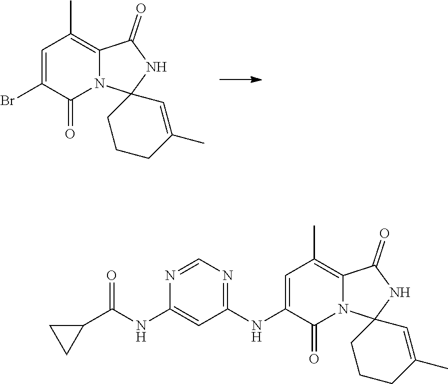 [SMILES: CC1=CC2(CCC1)NC(=O)c1c(C)cc(Br)c(=O)n12 ; CC1=CC2(CCC1)NC(=O)c1c(C)cc(Nc3cc(NC(=O)C4CC4)ncn3)c(=O)n12]

According to general procedure E, 6′-Bromo-3,8′-dimethyl-2′H-spiro[cyclohexane-1,3′-imidazo[1,5-a]pyridin]-2-ene-1′,5′-dione (60 mg, 0.186 mmol), N-(6-aminopyrimidin-4-yl)cyclopropanecarboxamide (40 mg, 0.223 mmol), Cs₂CO₃ (181 mg, 0.557 mmol), Xantphos (21 mg, 0.037 mmol), Pd(OAc)₂ (4.0 mg, 0.019 mmol), and 1,4-dioxane (4.0 mL) generated the title compound (15 mg, 0.035 mmol, 19%). ¹H NMR (400 MHz, CDCl₃) δ 8.54 (d, J=3.2 Hz, 2H), 8.30 (s, 1H), 7.67 (s, 1H), 6.89 (s, 1H), 5.60-5.55 (m, 1H), 3.84 (d, J=16.9 Hz, 1H), 3.18 (td, J=12.4, 6.8 Hz, 1H), 2.57 (s, 3H), 2.42 (d, J=18.2 Hz, 1H), 1.90-1.76 (comp, 3H), 1.72 (s, 3H), 1.66-1.60 (m, 1H), 1.12 (q, J=3.8 Hz, 2H), 0.95 (dq, J=7.4, 4.0 Hz, 2H). UHPLC-MS (ESI): Rt 1.15 min, m/z 421.4 [M]⁺.

### Example 33

### Synthesis of 6′-((6-Aminopyrimidin-4-yl)amino)-3,8′-dimethyl-2′H-spiro[cyclohexane-1,3′-imidazo[1,5-a]pyridin]-2-ene-1′,5′-dione (4ET-01-058)

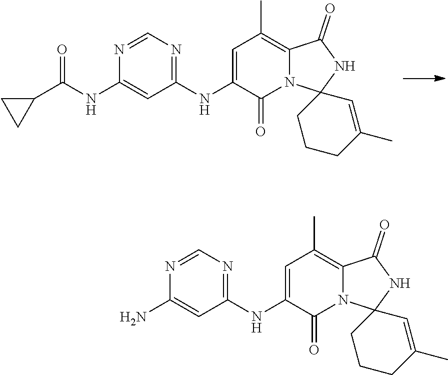 [SMILES: CC1=CC2(CCC1)NC(=O)c1c(C)cc(Nc3cc(NC(=O)C4CC4)ncn3)c(=O)n12 ; CC1=CC2(CCC1)NC(=O)c1c(C)cc(Nc3cc(N)ncn3)c(=O)n12]

According to general procedure F2, N-(6-((3,8′-Dimethyl-1′,5′-dioxo-1′,5′-dihydro-2′H-spiro[cyclohexane-1,3′-imidazo[1,5-a]pyridin]-2-en-6′-yl)amino)pyrimidin-4-yl)cyclopropanecarboxamide (10 mg, 0.024 mmol), 6N KOH aqueous solution (0.079 mL, 0.476 mmol) in EtOH/THF/H₂O (2 mL, v:v:v/2:1:1) generated the title compound (4ET-01-058) (7 mg, 0.020 mmol, 88%). 1H NMR (400 MHz, DMSO-d₆) δ 9.65 (s, 1H), 8.54 (s, 1H), 8.38 (s, 1H), 8.15 (s, 1H), 6.48 (s, 2H), 6.14 (d, J=1.0 Hz, 1H), 5.46 (s, 1H), 3.65 (d, J=17.0 Hz, 1H), 2.98 (td, J=12.2, 6.8 Hz, 1H), 2.41 (s, 3H), 2.27-2.17 (m, 2H), 1.77 (d, J=16.3 Hz, 1H), 1.64 (s, 3H), 1.47 (d, J=13.7 Hz, 1H); UHPLC-MS (ESI): Rt 0.95 min, m/z 353.3 [M+H]+.

### Example 34

### Synthesis of 6′-Bromo-3-ethyl-8′-methyl-2′H-spiro[cyclohexane-1,3′-imidazo[1,5-a]pyridin]-2-ene-1′,5′-dione

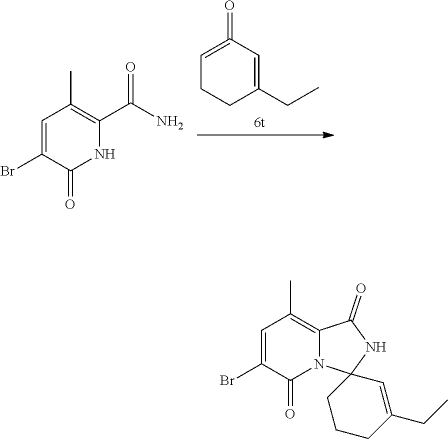

In general procedure A, compound 5 (200 mg, 0.86 mmol), 3-ethylcyclohex-2-en-1-one (6t, 322 mg, 2.60 mmol), H₂SO₄ (0.023 mL, 0.43 mmol), and 1,4-dioxane (8.0 mL) generated the title compound (65 mg, 0.19 mmol, 22%). ¹H NMR (400 MHz, DMSO-d₆) δ 10.04 (s, 1H), 8.04 (s, 1H), 5.46 (s, 1H), 3.61 (m, 1H), 2.95 (m, 1H), 2.39 (s, 3H), 2.25 (m, 2H), 1.97 (m, 2H), 1.81 (m, 1H), 1.55 (m, 1H), 0.96 (t, J=7.4 Hz, 3H). UHPLC-MS (ESI): Rt 0.78 min, m/z 337.1 [M]⁺.

### Example 35

### Synthesis of N-(6-((3-Ethyl-8′-methyl-1′,5′-dioxo-1′,5′-dihydro-2′H-spiro[cyclohexane-1,3′-imidazo[1,5-a]pyridin]-2-en-6′-yl)amino)pyrimidin-4-yl)cyclopropanecarboxamide (4ET-03-039)

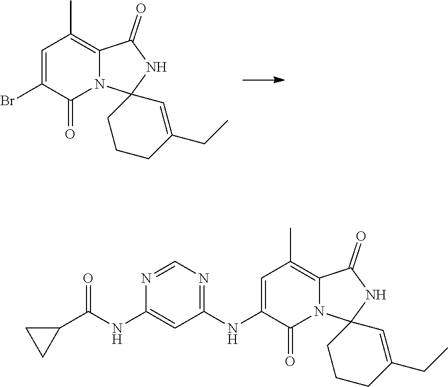 [SMILES: CCC1=CC2(CCC1)NC(=O)c1c(C)cc(Br)c(=O)n12 ; CCC1=CC2(CCC1)NC(=O)c1c(C)cc(Nc3cc(NC(=O)C4CC4)ncn3)c(=O)n12]

In general procedure E, compound 7h (63 mg, 0.18 mmol), N-(6-aminopyrimidin-4-yl)cyclopropanecarboxamide (50 mg, 0.28 mmol), Cs₂CO₃ (183 mg, 0.56 mmol), Xantphos (21.6 mg, 0.037 mmol), Pd(OAc)₂ (4.2 mg, 0.019 mmol), and 1,4-dioxane (2.0 mL) generated the title compound (4ET-03-039) (42 mg, 0.096 mmol, 53%). ¹H NMR (400 MHz, DMSO-d₆) δ 10.85 (s, 1H), 9.72 (br s, 1H), 9.12 (s, 1H), 8.53 (s, 1H), 8.49 (s, 1H), 7.85 (s, 1H), 5.48 (br s, 1H), 3.67 (m, 1H), 3.03 (m, 1H), 2.45 (s, 3H), 2.27 (m, 2H), 2.00 (m, 3H), 1.82 (m, 1H), 1.52 (m, 1H), 0.97 (t, J=7.4 Hz, 3H), 0.84 (d, J=6.2 Hz, 4H). UHPLC-MS (ESI): Rt 0.78 min, m/z 435.3 [M+H]⁺.

### Example 36

### Synthesis of 6′-((6-Aminopyrimidin-4-yl)amino)-3-ethyl-8′-methyl-2′H-spiro[cyclohexane-1,3′-imidazo[1,5-a]pyridin]-2-ene-1′,5′-dione (4ET-01-021)

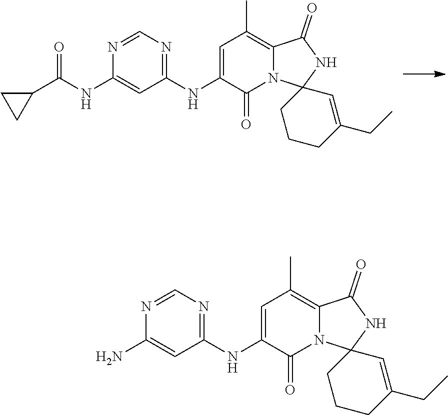 [SMILES: CCC1=CC2(CCC1)NC(=O)c1c(C)cc(Nc3cc(NC(=O)C4CC4)ncn3)c(=O)n12 ; CCC1=CC2(CCC1)NC(=O)c1c(C)cc(Nc3cc(N)ncn3)c(=O)n12]

In General Procedure F2, compound 4ET-03-039 (30 mg, 0.069 mmol), 6N KOH aqueous solution (0.060 mL, 0.35 mmol) in EtOH/THF/H₂O (2 mL, v:v:v/2:1:1) generated the title compound 12g (4ET-01-021) (18 mg, 0.049 mmol, 72%). ¹H NMR (400 MHz, DMSO-d₆) δ 9.66 (br, 1H), 8.57 (s, 1H), 8.41 (s, 1H), 8.17 (s, 1H), 6.50 (br, 2H), 6.16 (s, 1H), 5.48 (s, 1H), 3.70 (m, 1H), 3.03 (m, 1H), 2.43 (s, 3H), 2.27 (m, 2H), 1.97 (m, 2H), 1.82 (m, 1H), 1.50 (m, 1H), 0.97 (m, 3H). UHPLC-MS (ESI): Rt 0.65 min, m/z 367.3 [M+H]⁺.

### Example 37

### Synthesis of N-(6-((8′-Methyl-1′,5′-dioxo-1′,2,3,3a,4,5′,6,6a-octahydro-1H,2′H-spiro[cyclopenta[c]pyrrole-5,3′-imidazo[1,5-a]pyridin]-6′-yl)amino)pyrimidin-4-yl)cyclopropanecarboxamide (4ET-03-050A)

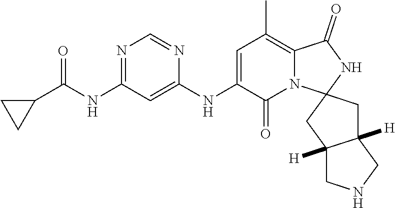 [SMILES: Cc1cc(Nc2cc(NC(=O)C3CC3)ncn2)c(=O)n2c1C(=O)NC21C[C@H]2CNC[C@H]2C1]

Second eluting isomer on reverse phase HPLC: ¹H NMR (400 MHz, DMSO-d₆) δ 10.85 (s, 1H), 9.18 (s, 1H), 8.55 (s, 1H), 8.50 (s, 1H), 7.85 (s, 1H), 3.15-2.98 (m, 8H), 2.42 (s, 3H), 2.05-1.99 (m, 1H), 1.95-1.80 (m, 2H), 0.82 (d, J=6.2 Hz, 4H); UHPLC-MS (ESI): Rt 0.61 min, m/z 436.3 [M+H]⁺.

### Example 38

### Synthesis of N-(6-((8′-Methyl-1′,5′-dioxo-1′,2,3,3a,4,5′,6,6a-octahydro-1H,2′H-spiro[cyclopenta[c]pyrrole-5,3′-imidazo[1,5-a]pyridin]-6′-yl)amino)pyrimidin-4-yl)cyclopropanecarboxamide (4ET-03-050B)

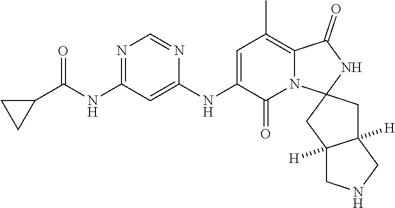 [SMILES: Cc1cc(Nc2cc(NC(=O)C3CC3)ncn2)c(=O)n2c1C(=O)NC21C[C@H]2CNC[C@H]2C1]

First eluting isomer on reverse phase HPLC): H NMR (400 MHz, DMSO-d₆) δ 10.85 (s, 1H), 9.18 (s, 1H), 8.55 (s, 1H), 8.51 (s, 1H), 7.86 (s, 1H), 3.55-3.40 (m, 4H), 3.10-3.04 (m, 2H), 3.10-2.95 (m, 2H), 2.42 (s, 3H), 2.04-1.98 (m, 1H), 1.95-1.81 (m, 2H), 0.84-0.78 (m, 4H). UHPLC-MS (ESI): Rt 0.60 min, m/z 436.3 [M+H]⁺.

### Example 39

### Synthesis of 6′-((6-Aminopyrimidin-4-yl)amino)-8′-methyl-3a,4,6,6a-tetrahydro-1H,2′H,3H-spiro[cyclopenta[c]furan-5,3′-imidazo[1,5-a]pyridine]-1′,5′-dione (4ET-01-014A)

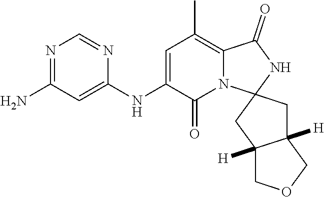 [SMILES: Cc1cc(Nc2cc(N)ncn2)c(=O)n2c1C(=O)NC21C[C@H]2COC[C@H]2C1]

Second eluting isomer on reverse phase HPLC): ¹H NMR (400 MHz, CDCl₃) δ 8.48 (s, 1H), 8.38 (s, 1H), 7.98 (s, 1H), 7.83 (s, 1H), 5.80 (s, 1H), 4.69 (br s, 2H), 3.93 (d, J=10.0 Hz, 2H), 3.75-3.65 (m, 4H), 3.48 (s, 2H), 3.15-3.02 (m, 2H), 2.55 (s, 3H); UHPLC-MS (ESI): Rt 0.61 min, m/z 369.3 [M+H]⁺.

### Example 40

### Synthesis of 6′-((6-Aminopyrimidin-4-yl)amino)-8′-methyl-3a,4,6,6a-tetrahydro-1H,2′H,3H-spiro[cyclopenta[c]furan-5,3′-imidazo[1,5-a]pyridine]-1′,5′-dione (4ET-01-014B)

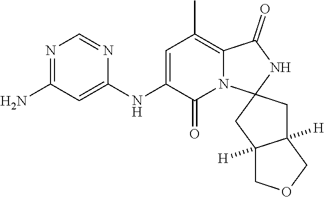 [SMILES: Cc1cc(Nc2cc(N)ncn2)c(=O)n2c1C(=O)NC21C[C@H]2COC[C@H]2C1]

First eluting isomer on reverse phase HPLC): ¹H NMR (400 MHz, CDCl₃) δ 8.48 (s, 1H), 8.38 (s, 1H), 7.98 (br s, 1H), 7.83 (br s, 1H), 5.80 (s, 1H), 4.69 (br s, 2H), 3.93 (d, J=10.0 Hz, 2H), 3.75-3.66 (m, 4H), 3.48 (s, 2H), 3.12-3.05 (m, 2H), 2.55 (s, 3H); UHPLC-MS (ESI): Rt 0.58 min, m/z 369.3 [M+H]⁺.

### Example 41

### Synthesis of N-(6-((1′-Fluoro-8-methyl-1,5-dioxo-1,1′,3′,5-tetrahydro-2H-spiro[imidazo[1,5-a]pyridine-3,2′-inden]-6-yl)amino)pyrimidin-4-yl)cyclopropanecarboxamide (4ET-03-052A)

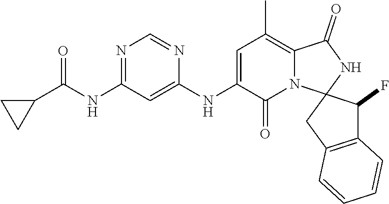 [SMILES: Cc1cc(Nc2cc(NC(=O)C3CC3)ncn2)c(=O)n2c1C(=O)NC21Cc2ccccc2[C@@H]1F]

First eluting atropisomer on reverse phase HPLC): ¹H NMR (400 MHz, DMSO-d₆) δ 10.76 (s, 1H), 9.78 (br s, 1H), 9.22 (s, 1H), 8.49 (s, 1H), 7.79 (s, 1H), 7.42-7.38 (m, 2H), 7.30-7.26 (m, 2H), 6.00 (d, J=55.2 Hz, 1H), 4.02 (d, J=17.6 Hz, 1H), 3.36-3.30 (m, 1H), 2.65 (s, 0.5H), 2.48 (s, 3H), 2.30 (s, 0.5H), 1.99-1.94 (m, 1H), 0.80-0.77 (d, J=5.6 Hz, 4H); UHPLC-MS (ESI): Rt 0.73 min, m/z 461.3 [M+H]⁺.

### Example 42

### Synthesis of N-(6-((1′-Fluoro-8-methyl-1,5-dioxo-1,1′,3′,5-tetrahydro-2H-spiro[imidazo[1,5-a]pyridine-3,2′-inden]-6-yl)amino)pyrimidin-4-yl)cyclopropanecarboxamide (4ET-03-052B)

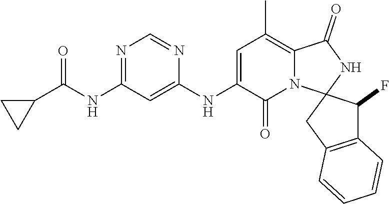 [SMILES: Cc1cc(Nc2cc(NC(=O)C3CC3)ncn2)c(=O)n2c1C(=O)NC21Cc2ccccc2[C@@H]1F]

Second eluting atropisomer on reverse phase HPLC): ¹H NMR (400 MHz, DMSO-d₆) δ 10.82 (s, 1H), 9.98 (br s, 1H) 9.16 (s, 1H), 8.52 (s, 1H), 7.81 (s, 1H), 7.47-7.33 (m, 4H), 6.58 (d, J=20.8 Hz, 1H), 4.19 (d, J=16.4 Hz, 1H), 3.30-3.12 (m, 1H), 2.56-2.45 (m, 1H), 2.47 (s, 3H), 2.01-1.96 (m, 1H), 0.80 (d, J=6.0 Hz, 4H); UHPLC-MS (ESI): Rt 3.3 min, m/z 461.3. [M+H]⁺.

### Example 43

### Synthesis of 6-((6-Aminopyrimidin-4-yl)amino)-1′-fluoro-8-methyl-1′,3′-dihydro-2H-spiro[imidazo[1,5-a]pyridine-3,2′-indene]-1,5-dione (4ET-01-010A)

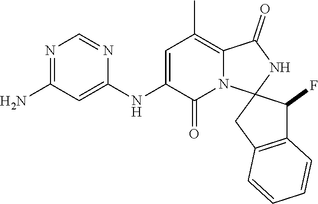 [SMILES: Cc1cc(Nc2cc(N)ncn2)c(=O)n2c1C(=O)NC21Cc2ccccc2[C@@H]1F]

Second eluting atropisomer on reverse phase HPLC): ¹H NMR (400 MHz, DMSO-d₆) δ 9.90 (br s, 1H), 8.61 (s, 1H), 8.48 (s, 1H), 8.17 (s, 1H), 7.50-7.17 (m, 4H), 6.49 (s, 2H), 6.12 (s, 1H), 4.20-4.12 (m, 1H), 3.24-3.19 (m, 2H), 2.42 (s, 3H); UHPLC-MS (ESI): Rt 0.64 min, m/z 393.2 [M+H]⁺.

### Example 44

### Synthesis of 6-((6-Aminopyrimidin-4-yl)amino)-1′-fluoro-8-methyl-1′,3′-dihydro-2H-spiro[imidazo[1,5-a]pyridine-3,2′-indene]-1,5-dione (4ET-01-010B)

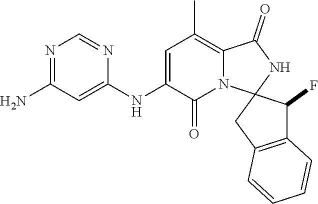 [SMILES: Cc1cc(Nc2cc(N)ncn2)c(=O)n2c1C(=O)NC21Cc2ccccc2[C@@H]1F]

First eluting atropisomer on reverse phase HPLC): ¹H NMR (400 MHz, DMSO-d₆) δ 8.60 (br s, 1H), 8.40 (s, 1H), 8.12 (s, 1H), 7.42-7.37 (m, 2H), 7.35-7.23 (m, 2H), 6.41 (s, 2H), 6.08 (s, 1H), 6.02 (br s, 0.5H), 5.88 (br s, 0.5H), 4.00 (d, J=16.0 Hz, 1H), 3.18-3.16 (m, 1H), 2.64-2.62 (m, 1H), 2.41 (s, 3H); UHPLC-MS (ESI): Rt 0.62 min, m/z 393.3 [M+H]⁺.

### Example 45

### Synthesis of N-(6-((1′-Hydroxy-8-methyl-1,5-dioxo-1,1′,3′,5-tetrahydro-2H-spiro[imidazo[1,5-a]pyridine-3,2′-inden]-6-yl)amino)pyrimidin-4-yl)cyclopropanecarboxamide (4ET-03-063)

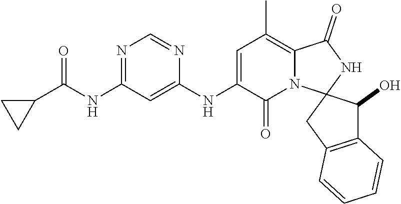 [SMILES: Cc1cc(Nc2cc(NC(=O)C3CC3)ncn2)c(=O)n2c1C(=O)NC21Cc2ccccc2[C@@H]1O]

¹H NMR (400 MHz, DMSO-d₆, one NH proton not detected) δ 10.81 (s, 1H), 8.18 (s, 1H), 8.56-8.50 (m, 2H), 7.88-7.72 (m, 2H), 7.72 (s, 1H), 7.68-7.62 (m, 1H), 7. 7.56-7.49 (m, 1H), 4.08 (d, J=12.0 Hz, 1H), 3.41 (d, J=12.0 Hz, 1H), 2.42 (s, 3H), 2.01-1.91 (m, 2H), 1.80-1.78 (m, 1H), 0.85-0.72 (m, 4H); UHPLC-MS (ESI): Rt 0.76 min, m/z 457.3 [M+H]⁺.

### Biological Example 1

### IC₅₀ Testing MNK Inhibitors

The ability of MNK inhibitors described herein to inhibit activity of MNK1 was tested using the recombinant human kinase, MNK1, in a substrate phosphorylation assay. IC₅₀ data is provided in Table 2 below. The ability of MNK inhibitors described herein to inhibit activity of MNK2 was tested using the recombinant human kinase, MNK2, in a substrate phosphorylation assay. IC₅₀ data is provided in Table 2 below.

The ability of MNK inhibitors described herein to inhibit eIF4E phosphorylation at Serine 209 in the human embryonic kidney (HEK) 293 cell line was tested by exposing the cells to compound for 2 hours and then measuring eIF4E phosphorylation with a phosphorylation-specific antibody in a fluorescent plate reader. IC₅₀ data is provided in Table 2. These experiments were done with HEK-293 cells plated on 96 well plates. Following treatment, cells were fixed with ice cold methanol for 10 min and then washed in 1× phosphate buffered saline (PBS) and the permeabilized with 0.02% Triton X-100 in 10% normal goat serum made up in PBS. Primary antibody was applied overnight at a concentration dilution of 1:2000 (p-eIF4E antibody from Cell Signaling ab76256). Following washing, cells were then exposed to secondary antibody conjugated to alexa-fluor 488 and then visualized on a Syngergy HTX plate reader. Fluorescence for p-eIF4E was measured and normalized to total DAPI fluorescence to determine the percentage of eIF4E phosphorylation in each well. Data were plotted in Graphpad Prism V8 to determine concentration-response effects and calculate IC₅₀ values. IC₅₀ data is provided in Table 2 below.

The ability of MNK inhibitors described herein to inhibit eIF4E phosphorylation at Serine 209 in Karpas 299, Human Non-Hodgkin's Ki-positive Large Cell Lymphoma cell line was tested by sandwich enzyme linked immune-absorbance assay. IC₅₀ data is provided in Table 2 below.

The ability of MNK inhibitors described herein to inhibit eIF4E phosphorylation at Serine 209 in the human osteosarcoma (U2OS) cell line was tested by exposing the cells to compound for 2 hours and then measuring eIF4E phosphorylation with a phosphorylation-specific antibody in a fluorescent plate reader. IC₅₀ data is provided in Table 2 below.

TABLE 2
MNK1, MNK2, HEK293 Cell, Karpas 299 Cell, and U2OS Cell IC₅₀
values for representative compounds of the disclosure

| | MNK1 | MNK2 | HEK 293 | Karpas 299 | U2OS cell |
|---|---|---|---|---|---|
| Compound | (nM) | (nM) | cell (nM) | cell (nM) | (nM) |
| 4ET-01-001 | 81.3 | 2.9 | 10.9 | — | — |
| 4ET-01-002 | 27.9 | 2.6 | 18.5 | 33 | 6.9 |
| 4ET-01-003 | 28.2 | 2.0 | 10.5 | 35 | — |
| 4ET-01-004 | 103.7 | 8.2 | 33.6 | — | — |
| 4ET-01-005 | 9.8 | 1.2 | 8.2 | 26 | 8.2 |
| 4ET-01-010A | 73.4 | 9.24 | 35.4 | — | — |
| 4ET-01-010B | 348 | 96.6 | 71.3 | — | — |
| 4ET-01-014A | 26.0 | 4.0 | 28.4 | — | — |
| 4ET-01-014B | 24.5 | 4.6 | 67.4 | — | — |
| 4ET-01-021 | 38.7 | 5.8 | 6.0 | — | — |
| 4ET-01-058 | 58.4 | 6.5 | 10.9 | — | — |
| 4ET-02-001 | 39.6 | 1.6 | 7.1 | 17 | — |
| 4ET-02-002 | inactive | inactive | — | — | — |
| 4ET-02-003 | 139.6 | 4.1 | 1.4 | — | — |
| 4ET-02-004 | 61.7 | 2.1 | 30.7 | — | 30 |
| 4ET-02-005 | 45.1 | 10.4 | 22.5 | — | — |
| 4ET-02-006 | 346.0 | 14.4 | — | — | — |
| 4ET-02-007 | 89.0 | 3.6 | — | — | — |
| 4ET-03-001 | 118.6 | 11.9 | 47.0 | — | — |
| 4ET-03-002 | 133.0 | 6.5 | 5.3 | — | — |
| 4ET-03-003 | 80.2 | 22.6 | 110.2 | — | — |
| 4ET-03-004 | 84.5 | 7.7 | 86.6 | — | — |
| 4ET-03-005 | 20.5 | 1.8 | 2.6 | 6.4 | 5.1 |
| 4ET-03-006 | 64.6 | 5.9 | 53 | — | — |
| 4ET-03-007 | 96.7 | 4.9 | 10.0 | — | — |
| 4ET-03-008 | 65.5 | 7.4 | 52.6 | — | — |
| 4ET-03-009 | 31.3 | 2.6 | 1.7 | 6.2 | 1.7 |
| 4ET-03-010 | 381.8 | 84.8 | 164.1 | — | |
| 4ET-03-011 | 203.3 | 27.8 | 32.4 | — | — |
| 4ET-03-012 | 441.4 | 273.2 | — | — | — |
| 4ET-03-013 | 204.6 | 8.0 | inactive | — | inactive |
| 4ET-03-014 | 107.0 | 32.8 | — | — | — |
| 4ET-03-015 | 19.1 | 1.4 | 2.7 | 3.0 | 2.4 |
| 4ET-03-016 | — | — | — | — | — |
| 4ET-03-017 | 74.3 | 3.0 | 0.9 | 1.0 | 0.4 |
| 4ET-03-035 | 128.1 | 4.30 | 8.7 | — | — |
| 4ET-03-036 | 117.8 | 5.65 | 8.6 | — | — |
| 4ET-03-039 | 31.6 | 1.62 | 2.6 | — | — |
| 4ET-03-050A | 35.0 | 1.32 | 2.6 | — | — |
| 4ET-03-050B | 40.9 | 6.84 | 149.9 | — | — |
| 4ET-03-052A | 4.71 | 0.50 | 19.2 | — | — |
| 4ET-03-052B | 19.9 | 1.63 | 11.7 | — | — |
| 4ET-03-054 | 117.8 | 5.7 | 8.6 | — | — |
| 4ET-03-063 | 35.0 | 3.0 | 17.0 | — | — |
| 4ET-03-066A | 47.9 | 3.4 | 6.5 | — | — |

— indicates compound was not tested

It was observed that many of the compounds had similar inhibition across multiple cell lines.

### Biological Example 2

### Pharmacokinetic Study—Compound 4ET-03-009 at 10 mg/kg

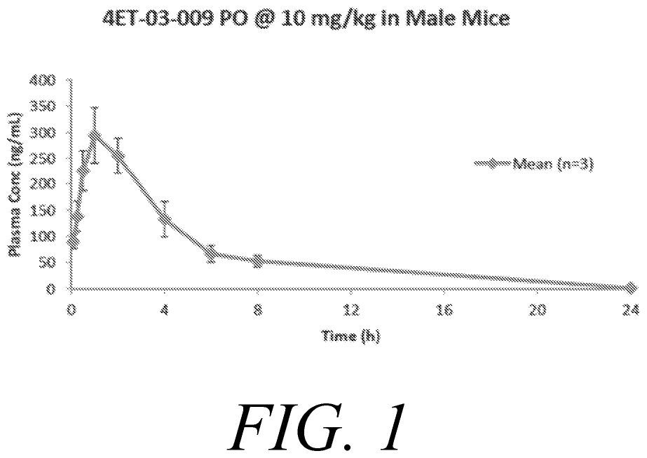

Compound 4ET-03-009 was dissolved in a 10% dimethylacetamide (DMA)/3000 polyethylene glycol (PEG) 300/400% propylene glycol (PG)/200% water solvent. CD-1 male mice were orally dosed with 10 mg/kg of compound 4ET-03-009 in 20 mL/kg of liquid. Plasma was collected at indicated timepoints and plasma concentration of compound 4ET-03-009 was measured using liquid chromatography-mass spectrometry/mass spectrometry (LC-MS/MS). Results are presented in FIG. 1 and Tables 3, 4, and 5. No abnormal clinical symptoms were observed during the study. T_{1/2} for the study was 3.5 hours, Cₘₐₓ was 716 nM, and AUC_{inf} was 1.62 hours*μg/mL.

TABLE 3
Plasma concentration of compound 4ET-
03-009 at various time points.

| Time | Plasma Conc. (ng/mL) | | | Mean | SD | CV |
|---|---|---|---|---|---|---|
| (hours) | (per mouse n = 3) | | | (ng/mL) | ng/mL | (%) |
| 0.083 | 79.8 | 85.3 | 106 | 90.4 | 13.8 | 15.3 |
| 0.25 | 106 | 147 | 161 | 138 | 28.6 | 20.7 |
| 0.5 | 202 | 270 | 205 | 226 | 38.4 | 17.0 |
| 1 | 331 | 318 | 233 | 294 | 53.2 | 18.1 |
| 2 | 227 | 271 | 216 | 255 | 33.6 | 13.2 |
| 4 | 104 | 169 | 124 | 132 | 33.3 | 25.2 |
| 6 | 49.3 | 80.2 | 70.6 | 66.7 | 15.8 | 23.7 |
| 8 | 40.6 | 62.9 | 53.9 | 52.5 | 11.2 | 21.4 |
| 24 | 1.64 | 2.28 | 1.84 | 1.92 | 0.327 | 17.1 |

SD = standard deviation, CV = percent coefficient of variation.

TABLE 4
Pharmacokinetic data for compound 4ET-03-009

| | t_{1/2} | Tₘₐₓ | Cₘₐₓ | C_{max/D} | Tₗₐₛₜ | Cₗₐₛₜ |
|---|---|---|---|---|---|---|
| Animal # | (hours) | (hours) | (ng/mL) | (kg/kL) | (hours) | (ng/mL) |
| 1 | 3.58 | 1.00 | 331 | 33.1 | 24.0 | 1.64 |
| 2 | 3.44 | 1.00 | 318 | 31.8 | 24.0 | 2.28 |
| 3 | 3.37 | 1.00 | 233 | 23.3 | 24.0 | 1.84 |
| Mean | 3.46 | 1.00 | 294 | 29.4 | 24.0 | 1.92 |
| (n = 3) | | | | | | |
| SD | 0.110 | 0 | 53.2 | 5.32 | 0 | 0.327 |
| % CV | 3.16 | 0 | 18.1 | 18.1 | 0 | 17.1 |

TABLE 5
Pharmacokinetic data for compound 4ET-03-009

| | AUCₗₐₛₜ | AUC_{inf} | AUC_{inf}/D | | AUC_{0-8 hours} |
|---|---|---|---|---|---|
| | (hours* | (hours* | (hours* | MRT_{inf} | (hours* |
| Animal # | ng/mL) | ng/mL) | kg/kL) | (hours) | ng/mL) |
| 1 | 1457 | 1465 | 147 | 4.12 | 1119 |
| 2 | 1870 | 1882 | 188 | 4.64 | 1349 |
| 3 | 1511 | 1520 | 152 | 4.74 | 1066 |
| Mean | 1613 | 1622 | 162 | 4.50 | 1178 |
| (n = 3) | | | | | |
| SD | 225 | 226 | 22.6 | 0.332 | 151 |
| % CV | 13.9 | 13.9 | 13.9 | 7.38 | 12.8 |

In Table 4 and 5 the abbreviations have the following meanings
SD=standard deviation
CV=percent coefficient of variation
t_{1/2}=time taken for half the initial dose of medicine administered to be eliminated from the body
Tₘₐₓ=time to reach Cₘₐₓ
Cₘₐₓ=maximum serum concentration
Cₘₐₓ/D=dose normalized Cₘₐₓ
Tₗₐₛₜ=time of last measurable concentration
Cₗₐₛₜ=last measurable plasma concentration
AUCₗₐₛₜ=area under the plasma concentration-time curve from time zero to time of last measurable concentration
AUC_{inf}=area under the plasma concentration-time curve from time zero to infinity
AUC_{inf}/D=dose normalized AUC_{inf}
MRT_{inf}=mean residence time to infinity
AUC_{0-8 hours}=area under the plasma concentration-time curve from time zero to 8 hours

It is therefore reasonable to conclude that MNK inhibitors of the present disclosure may safely be administered in a dose of 10 mg/kg. The plasma concentrations show that MNK inhibition will likely be achieved for 24 hrs because the plasma concentration at that time point would be expected to substantially inhibit MNK given the IC₅₀ of the compound.

In separate mice administered the same therapy, brain tissue was harvested 2 hours after dosing and brain concentration of the MNK inhibitor was measured by homogenizing brain tissue followed by LC-MS/MS analysis. Plasma concentration was measured as described above. Brain concentration and plasma ratio and other measured parameters are presented in Table 6. The data show that the compound enters the brain very poorly and can be considered to be peripherally restricted.

TABLE 6
Comparison data of plasma vs. brain of 4ET-03-009

| | | | | | | | | Mean |
|---|---|---|---|---|---|---|---|---|
| | Plasma or Tissue | | | | Mean | | | Tissue/ |
| | Homogenate Conc. | | | Mean | Tissue | SD | CV | Plasma |
| Matrix | (ng/mL) n = 3 | | | (ng/mL) | (ng/g) | (ng/mL) | (%) | Conc. Ratio |
| Plasma | 357 | 292 | 337 | 329 | n/a | 33.3 | 10.1 | n/a |
| Brain | 7.67 | 5.5 | 7.28 | 6.82 | 27.3 | 1.16 | 17.0 | 0.083 |

SD = standard deviation
CV = percent coefficient of variation

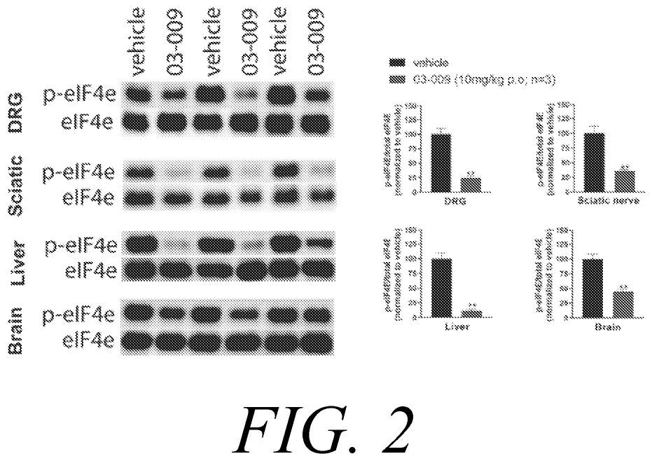

In separate mice administered the same therapy, 2 hours after dosing tissues were harvested from the sciatic nerve, liver, brain, and DRG and homogenized, then subjected to Western blot analysis for eIF4E and p-eIF4E. Control mice administered only the solvent vehicle with no MNK inhibitor were also assayed. Results are presented in FIG. 2, left panel. Amounts of p-eIF4E in each tissue based on the mean for all three mice administered the therapeutic and based on the mean for all three control mice are presented in FIG. 2, right panel. The MNK inhibitor significantly decreased peIF4E in all tissues assayed but the effect was greater in peripheral tissues than in the brain, as predicted by data in Table 6. Units on the y-axis are % of signal standardized to the control. Stars signify significant differences from vehicle treatment with a 2-way analysis of variance (ANOVA)test.

### Biological Example 3

### Pharmacokinetic Study—Compound 4ET-03-009 at 20 mg/kg

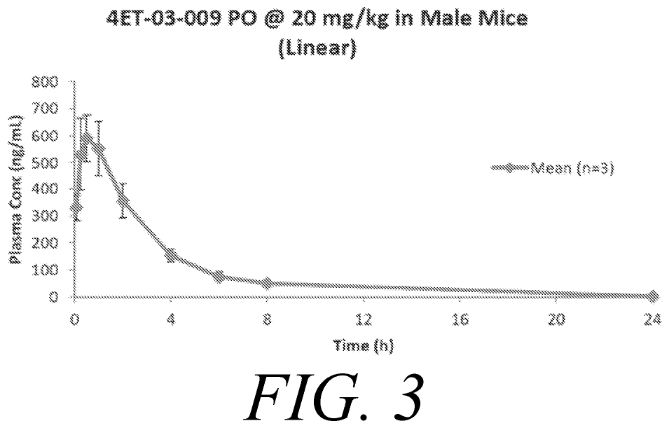

Compound 4ET-03-009 was dissolved in a 10% dimethylacetamide (DMA)/30% polyethylene glycol (PEG) 300/40% propylene glycol (PG)/20% water solvent. Dosing and assay were performed as described in Biological Example 2. Results are presented in FIG. 3 and Tables 7, 8, and 9, below. No abnormal clinical symptoms were observed during the study. T_{1/2} for the study was 3.6 hours, Cₘₐₓ was 1.4 μM, and AUC_{inf} was 2.26 hours*μg/mL.

TABLE 7
Plasma concentration of compound 4ET-
03-009 at various time points.

| Time | Plasma Conc. (ng/mL) | | | Mean | SD | CV |
|---|---|---|---|---|---|---|
| (hours) | (per mouse n = 3) | | | (ng/mL) | ng/mL | (%) |
| 0.083 | 304 | 384 | 304 | 331 | 46.2 | 14.0 |
| 0.25 | 665 | 529 | 396 | 530 | 135 | 25.4 |
| 0.5 | 666 | 607 | 497 | 590 | 85.8 | 14.5 |
| 1 | 649 | 561 | 448 | 553 | 101 | 18.2 |
| 2 | 408 | 372 | 286 | 355 | 62.7 | 17.6 |
| 4 | 180 | 144 | 135 | 153 | 23.8 | 15.6 |
| 6 | 93.6 | 71.3 | 58.7 | 74.5 | 17.7 | 23.7 |
| 8 | 57.6 | 51.4 | 41.5 | 50.2 | 8.12 | 16.2 |
| 24 | 2.75 | 2.01 | 1.89 | 2.22 | 0.466 | 21.0 |

SD = standard deviation,
CV = percent coefficient of variation

TABLE 8
Pharmacokinetic data for compound 4ET-03-009

| | t_{1/2} | Tₘₐₓ | Cₘₐₓ | Cₘₐₓ/D | Tₗₐₛₜ | Cₗₐₛₜ |
|---|---|---|---|---|---|---|
| Animal # | (hours) | (hours) | (ng/mL) | (kg/kL) | (hours) | (ng/mL) |
| 1 | 3.58 | 0.500 | 666 | 33.3 | 24.0 | 2.75 |
| 2 | 3.47 | 0.500 | 607 | 30.4 | 24.0 | 2.01 |
| 3 | 3.61 | 0.500 | 497 | 24.9 | 24.0 | 1.89 |
| Mean | 3.55 | 0.500 | 590 | 29.5 | 24.0 | 2.22 |
| (n = 3) | | | | | | |
| SD | 0.077 | 0 | 85.8 | 4.29 | 0 | 0.466 |
| % CV | 2.17 | 0 | 14.5 | 14.5 | 0 | 21.0 |

TABLE 9
Pharmacokinetic data for compound 4ET-03-009

| | AUCₗₐₛₜ | AUC_{inf} | AUC_{inf}/D | | AUC_{0-8 hours} |
|---|---|---|---|---|---|
| | (hours* | (hours* | (hours* | MRT_{inf} | (hours* |
| Animal # | ng/mL) | ng/mL) | kg/kL) | (hours) | ng/ml) |
| 1 | 2613 | 2627 | 131 | 3.63 | 2130 |
| 2 | 2274 | 2284 | 114 | 3.53 | 1847 |
| 3 | 1848 | 1858 | 92.9 | 3.63 | 1501 |
| Mean | 2245 | 2256 | 113 | 3.59 | 1826 |
| (n = 3) | | | | | |
| SD | 383 | 385 | 19.3 | 0.053 | 315 |
| % CV | 17.1 | 17.1 | 17.1 | 1.48 | 17.3 |

SD = standard deviation
CV = percent coefficient of variation
t_{1/2} = time taken for half the initial dose of medicine administered to be eliminated from the body;
Tₘₐₓ = time to reach Cₘₐₓ;
Cₘₐₓ = maximum serum concentration;
Cₘₐₓ/D = dose normalized Cₘₐₓ;
Tₗₐₛₜ = time of last measurable concentration;
Cₗₐₛₜ = last measurable plasma concentration;
AUCₗₐₛₜ = area under the plasma concentration-time curve from time zero to time of last measurable concentration;
AUC_{inf} = area under the plasma concentration-time curve fromtime zero to infinity;
AUC_{inf}/D = dose normalized AUC_{inf};
MRT_{inf} = mean residence timeto infinity;
AUC_{0-8 hours} = area under the plasma concentration-time curve from time zero to 8 hours

### Biological Example 4

### Blood Brain Barrier Permeability

Permeability of the blood-brain barrier to various MNK inhibitors of the present disclosure and eFT508 as a comparison was evaluated using an in vitro drug and metabolism pharmacokinetic (DMPK) study. In particular, the study was conducted in Madin Darby Canine Kidney (MDCK) cells that express the MDR1 gene (ABCB1) that encodes for the efflux protein, P-gp. MDCK-MDR1 is a stable-transfected cell line originating from MDCK cells, with over-expression of human MDR1 gene. Because MDCK-MDR1 permeability correlates well with brain exposure it is often utilized predictor of blood-brain-barrier penetration.

In the assay, test compounds were evaluated at 5 μM and the average of two experiments reported for Papp A-B and Papp B-A. Analysis was performed using LC-MS/MS. Apparent permeability (Papp) values are calculated using the following equation:

Papp=(dQ/dt)/A/C0

where dQ/dt is the initial rate of amount of test compound transported across cell monolayer, A is the surface area of the filter membrane, and C₀ is the initial concentration of the test compound, calculated for each direction using a 4-point calibration curve by LC-MS/MS. Net flux ratio between the two directional transports was calculated by the following equation:

Ratio=Papp,B−A/Papp,A−B

where Papp, B−A and Papp, A−B represent the apparent permeability of test compound from the basal to apical and apical to basal side of the cellular monolayer, respectively. Results are presented in Table 10, below.

TABLE 10
Results for permeability testing of representative compounds

| | Pₐₚₚ | | Efflux |
|---|---|---|---|
| | A-B × ¹⁰⁻⁶ | Pₐₚₚ B-A × ¹⁰⁻⁶ | ratio (ratio |
| Compound | cm/s^{#} | cm/s*^{#} | B-A/A-B) |
| eFT508 | 26.8 | 41.7 | 1.6 |
| 4ET-01-002 | 2.6 | 5.6 | 2.2 |
| 4ET-01-003 | 9.1 | 13.3 | 1.5 |
| 4ET-01-004 | 0.1 | 0.3 | 4.4 |
| 4ET-01-005 | 0.2 | 0.6 | 2.7 |
| 4ET-01-010A | 42.7 | 63.9 | 1.5 |
| 4ET-01-014A | 24.9 | 46.4 | 1.9 |
| 4ET-01-014B | 2.4 | 5.8 | 2.4 |
| 4ET-01-021 | 55.3 | 52.0 | 0.9 |
| 4ET-01-058 | 57.8 | 61.2 | 1.1 |
| 4ET-02-004 | 0.3 | 7.6 | 30.2 |
| 4ET-02-005 | 4.5 | 42.2 | 9.4 |
| 4ET-02-006 | 0.1 | 0.1 | 0.8 |
| 4ET-02-007 | 7.3 | 40.8 | 5.6 |
| 4ET-03-005 | 15.5 | 58.6 | 3.8 |
| 4ET-03-007 | 2.4 | 15.1 | 6.3 |
| 4ET-03-009 | 5.4 | 25.0 | 4.6 |
| 4ET-03-013 | 0.2 | 3.9 | 18.1 |
| 4ET-03-015 | 0.9 | 8.7 | 10.0 |
| 4ET-03-017 | 16.3 | 58.9 | 3.6 |
| 4ET-03-050A | 2.0 | 24.5 | 12.4 |
| 4ET-03-050B | 0.7 | 0.9 | 1.3 |
| 4ET-03-052A | 12.1 | 80.6 | 6.6 |
| 4ET-03-052B | 22.9 | 63.3 | 2.8 |
| 4ET-03-063 | 8.7 | 64.9 | 10.2 |

^{#}Apical to basolateral transport; *^{#}Basolateral to apical transport

### Biological Example 5

### Liver Microsome Stability

Liver microsome stability of MNK inhibitors of the present disclosure and eFT508 as a comparison was tested to assess half-life (T_{1/2}) and intrinsic clearance (CLᵢₙₜ) in both rodent and human liver microsomes. T_{1/2} and CLᵢₙₜ from in vitro liver microsomal assays are used to predict rate of metabolism in the liver. Compounds that are rapidly metabolized by liver microsomes are predicted to have limited systemic exposure and poor oral bioavailability. Results are presented in Table 11a.

TABLE 11a
Liver microsome stability of representative compounds

| | Rodent liver | Human liver | CLᵢₙₜ rodent | CLᵢₙₜ human |
|---|---|---|---|---|
| | microsomes t_{1/2} | microsomes t_{1/2} | (μL/ | (μL/ |
| Compound | (min) | (min) | min/mg) | min/mg) |
| eFT508 | 484^{#} | 737 | 2.86 | 1.88 |
| 4ET-01-002 | 514^{#} | 639 | 2.70 | 2.17 |
| 4ET-01-003 | 596^{#} | 719 | 2.33 | 1.93 |
| 4ET-01-004 | 145^{#} | 541 | 9.57 | 2.56 |
| 4ET-01-005 | 393* | 385 | 3.53 | 3.60 |
| 4ET-02-001 | 546^{#} | 552 | 2.54 | 2.51 |
| 4ET-02-003 | 370^{#} | 599 | 3.75 | 2.31 |
| 4ET-02-004 | 555* | 1112 | 2.50 | 1.25 |
| 4ET-02-005 | 102^{#} | 246 | 13.6 | 5.64 |
| 4ET-02-007 | 159* | 216 | 8.70 | 6.42 |
| 4ET-03-005 | 287^{#} | 1793 | 4.84 | 0.77 |
| 4ET-03-009 | 545^{#} | 643 | 2.54 | 2.16 |
| 4ET-03-015 | 436^{#} | 507 | 3.18 | 2.73 |

^{#}Rat liver microsomes;
*Mouse liver microsomes;
t_{1/2} = the half-life, where t_{1/2} is equal to 0.693/slope;
CLᵢₙₜ = the intrinsic hepatic clearance (μL/min/mg), where CLᵢₙₜ is equal to 0.693/(t_{1/2} × Cmp);
Cmp = microsomal protein concentration (mg/mL).

Liver microsome stability of MNK inhibitors of the present disclosure and eFT508 as a comparison was tested to assess half-life (T_{1/2}) in mouse liver microsomes. T_{1/2} from in vitro liver microsomal assays are used to predict rate of metabolism in the liver. Compounds that are rapidly metabolized by liver microsomes are predicted to have limited systemic exposure and poor oral bioavailability. Results are presented in Table 11b.

TABLE 11b
Liver microsome stability of representative compounds

| Compound | T_{1/2} (min) |
|---|---|
| eFT508 | 484 |
| 4ET-01-010A | 115 |
| 4ET-01-014A | 270 |
| 4ET-01-014B | 430 |
| 4ET-01-021 | 93 |
| 4ET-01-058 | 460 |
| 4ET-03-050A | 72 |
| 4ET-03-050B | 424 |
| 4ET-03-052A | 110 |
| 4ET-03-052B | 173 |
| 4ET-03-063 | 222 |

t_{1/2} = the half-life, where t_{1/2} is equal to 0.693/slope

### Biological Example 6

### Off-Target Kinase Screen

Specificity of MNK inhibitors of the present disclosure and eFT508 as a control was determined by measuring the effects of 1 μM test compound on kinase activity of Cdc2-like kinase 4 (CLK4), death-associated protein kinase-related apoptosis-inducing protein kinase 1 (DRAK1), and protein kinase G 2 (PKG2). These kinases were chosen because they are the only known off-target hit kinases for eFT508. These assays were done using recombinant human kinases (CLK4, DRAK1 and PKG2) in a substrate phosphorylation assay. % activity remaining after MNK inhibitor treatment is indicated in Table 12. These results indicate that some MNK inhibitors are highly specific, while others have significant effects on the activity of non-MNK kinases.

TABLE 12
Results for off-target kinase screen

| Compound | CLK4 | DRAK1 | PKG2 |
|---|---|---|---|
| eFT508 | 68 | 24 | 100 |
| 4ET-01-002 | 100 | 72 | 97 |
| 4ET-01-003 | 91 | 65 | 88 |
| 4ET-01-004 | 93 | 76 | 100 |
| 4ET-01-005 | 63 | 41 | 100 |
| 4ET-02-001 | 92 | 81 | 99 |
| 4ET-02-004 | 81 | 86 | 100 |
| 4ET-03-009 | 54 | 16 | 99 |
| 4ET-03-015 | 14 | 16 | 100 |
| 4ET-03-017 | 17 | 9 | 100 |

### Biological Example 7

### Pharmacokinetic Study—Compound 4ET-01-021 in CD-1 Male Mice with IV and PO Dosing

4ET-01-021 was dissolved in 10% dimethylacetamide (DMA)/90% propylene glycol (PG) for oral dosing and 10% DMI (dimethyl isosorbide)/15% EtOH (ethanol)/35% PG (propylene glycol)/40% NS (normal saline) for IV dosing. CD-1 male mice were dosed with 4ET-01-021 at 1.0 mg/kg (IV), 3.0 mg/kg (PO), or 10.0 mg/kg (PO). Plasma was collected at indicated timepoints and plasma concentration of 4ET-01-021 was measured using liquid chromatography-mass spectrometry/mass spectrometry (LC-MS/MS). Results are presented in Tables 13-16.

In mice, following IV administration with 1.0 mg/kg, plasma concentrations declined in a multiphasic manner with an initial concentration (Cₒ) of 0.860 μg/mL and a last measurable concentration (Cₗₐₛₜ) of 19.3 ng/mL at 24 h post dose. The compound displayed a low systemic clearance (CLₚ) of 6.97 mL/min/kg and a high steady-state volume of distribution (Vₛₛ) of 3.49 L/kg, suggesting insignificant metabolism and extensive tissue distribution. The total systemic exposure (AUC_{inf}) was 2.42 h*μg/mL with a terminal half-life (t_{1/2}) of 8.42 hr. Results are presented in Table 13; mean values averaged for 3 animals are shown.

TABLE 13
Results for CD-1 dosed via IV at 1.0 mg/kg (5.0 mL/kg)

| t_{1/2} | C₀ | Tₗₐₛₜ | Cₗₐₛₜ |
|---|---|---|---|
| (hr) | (ng/ml) | (hr) | (ng/ml) |
| 8.42 | 860 | 24.0 | 19.3 |
| AUCₗₐₛₜ | AUC_{inf} | AUC_{onf}/D | V_{z} |
| (hr · ng/mL) | (hr · ng/mL) | (hr · mg/mL) | (L/kg) |
| 2190 | 2422 | 2422 | 5.10 |
| CLₚ | MRT_{inf} | V₅₅ | |
| (mL/min/kg) | (hr) | (L/kg) | — |
| 6.97 | 8.32 | 3.49 | — |

Following oral administration to mice at 3 mg/kg, the compound reached its high peak plasma concentration (Cₘₐₓ) of 643 ng/mL within 30 min. After that, its plasma concentrations declined in a multiphasic manner with a last measurable concentration of 36.3 ng/mL at 24 h and a terminal half-life (t_{1/2}) of 7.11 hr. The total systemic exposure (AUC_{inf}) was 4.50 h*μg/mL with an oral bioavailability of 61.9%. Results are presented in Table 14; mean values averaged for 3 animals are shown.

TABLE 14
Results for CD-1mice orally dosed at 3 mg/kg (10.0 mL/kg)

| t_{1/2} | Cₘₐₓ | Tₘₐₓ | Tₗₐₛₜ | Cₗₐₛₜ |
|---|---|---|---|---|
| (hr) | (ng/ml) | (hr) | (hr) | (ng/ml) |
| 7.11 | 643 | 0.5 | 24 | 36.3 |
| AUCₗₐₛₜ | AUC_{inf} | AUC_{inf}/D | MRT_{inf} | |
| (hr · ng/mL) | (hr · ng/ml) | (hr*kg/kL) | (hr) | F (%) |
| 4130 | 4501 | 1500 | 8.41 | 61.9 |

Following oral administration at 10 mg/kg, the compound rapidly reached its high peak plasma concentration (Cₘₐₓ) of 1,903 ng/mL within 15 min. After that, its plasma concentrations declined in a multiphasic manner with a last measurable concentration of 99.5 ng/mL at 24 h and a terminal half-life (t_{1/2}) of 7.44 h. The total systemic exposure (AUC_{inf}) was 12.0 hr·μg/mL with an oral bioavailability of 49.6%. Results are presented in Table 15; mean values averaged for 3 animals are shown.

TABLE 15
Results for CD1 mice orally dosed at 10 mg/kg (10.0 mL/kg)

| t_{1/2} | Cₘₐₓ | Tmax | Tₗₐₛₜ | Cₗₐₛₜ |
|---|---|---|---|---|
| (hr) | (ng/mL) | (hr) | (hr) | (ng/ml) |
| 7.44 | 1903 | 0.25 | 24 | 99.5 |
| AUCₗₐₛₜ | AUC_{inf} | AUC_{inf}/D | MRT_{inf} | |
| (hr · ng/mL) | (hr · ng/ml) | (hr*kg/kL) | (hr) | F (%) |
| 10961 | 12016 | 1202 | 8.51 | 49.6 |

4ET-01-021 has a brain to plasma ratio of 0.142 (B:P ratio=0.142) 2 h post PO dosing in mice at 3.0 mg/kg, as shown in Table 16, below.

TABLE 16a
3.0 mg/kg PO dose in male CD-1 mouse

| | | Time | Plasma/Tissue Homogenate | | |
|---|---|---|---|---|---|
| Analyte | Matrix | (hr) | Concentration (ng/ml) | | |
| 4ET- | Plasma | 2 | 404 | 462 | 383 |
| 01-021 | Brain | 2 | 14.5 | 17.6 | 12.2 |

TABLE 16b
Mean values for 3.0 mg/kg PO dose in male CD-1 mouse

| | | | | Mean tissue/ |
|---|---|---|---|---|
| | | | Mean | plasma |
| | | Mean | Tissue | concentration |
| Analyte | Matrix | (ng/mL) | (ng/g) | ratio |
| 4ET- | Plasma | 416 | n/a | n/a |
| 01-021 | Brain | 14.8 | 59.1 | 0.142 |

### Biological Example 8

### In Vivo Efficacy Testing—IL-6 Evoked Grimace Test

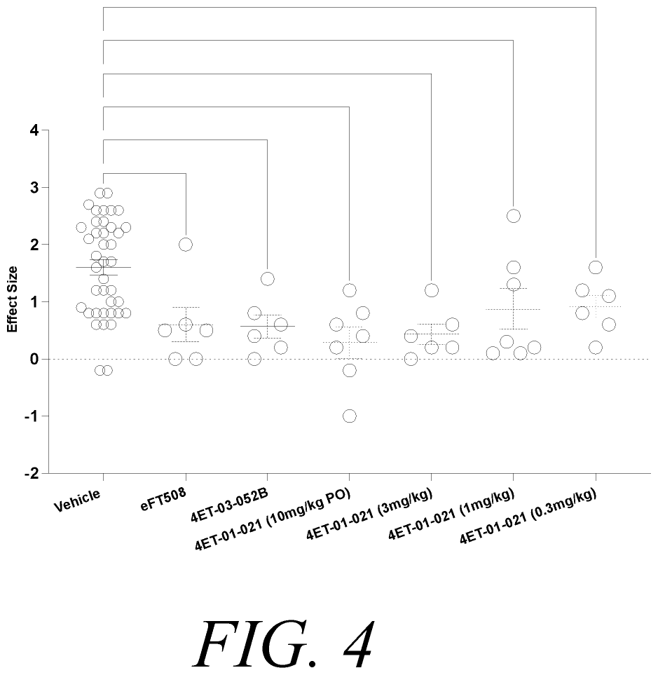

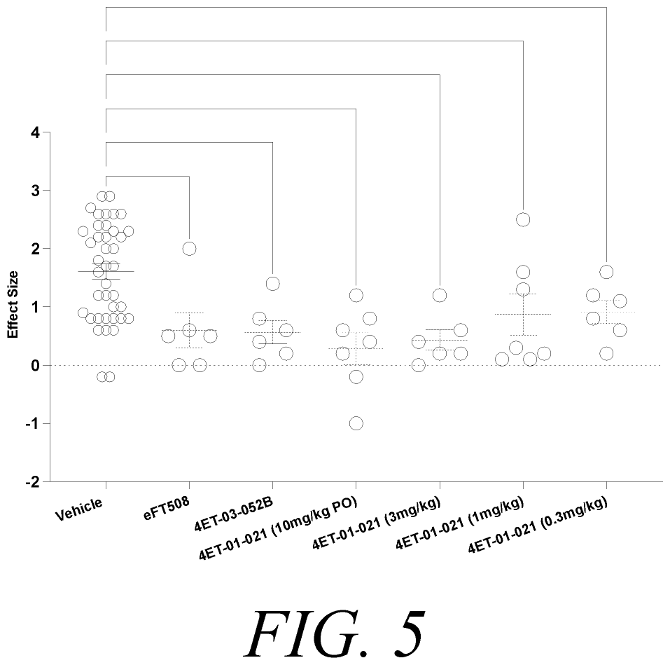

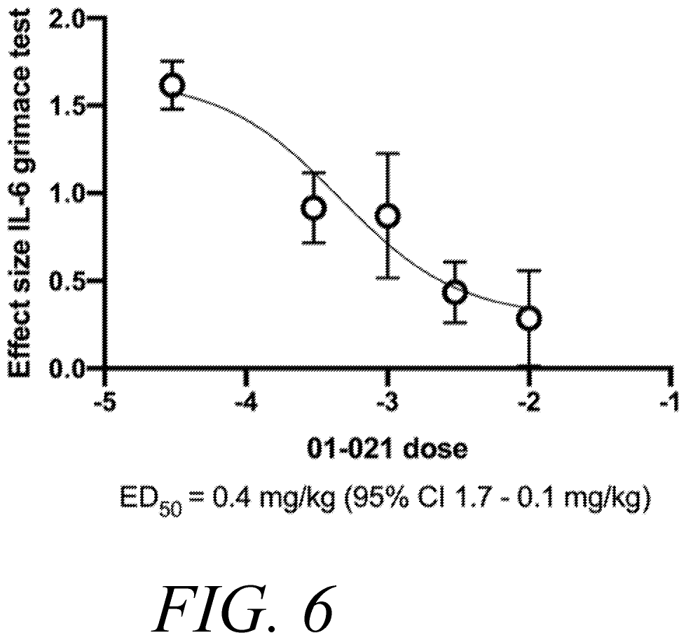

FIG. 4 shows evaluation of compounds in the IL-6 evoked grimace test. Institute for Cancer Research (ICR) mice were used in these experiments. Mice were bred in house at University of Texas at Dallas and used at between 12 and 24 weeks of age. Mice were habituated to plexiglass boxes of approximately 4×6 inches in size with openable tops. Habituation took place over 2 days with animals exposed to the boxes for at least 30 min. A blinded observer scored baseline grimacing using the method previously described by Mogil and colleagues (Langford, et al., Coding of facial expressions of pain in the laboratory mouse. Nat Methods (2010) 7:447-449.). On test day, mice were given test compounds 1 hr prior to injection of interleukin 6 (human recombinant IL-6, R&D Systems) which was given at a dose of 0.1 ng in saline via an intraplantar injection as described previously (Moy et al. The MNK-eIF4E Signaling Axis Contributes to Injury-Induced Nociceptive Plasticity and the Development of Chronic Pain. J Neurosci (2017) 37:7481-7499.). Test compounds were given orally (PO) via a flexible oral gavage canula built for mice. 4ET-01-021 (10.0, 3.0, and 1.0 mg/kg) and 4ET-03-052B (10.0 mg/kg) are efficacious in the IL-6 evoked grimace test in mice. FIG. 5 depicts a comparison of effect size in the IL-6 evoked grimace test. Effect sizes were calculated per mouse by subtracting the baseline grimace score from the sum of the grimace scores for 1 and 3 hr time points. 4ET-01-021 and 4ET-03-052B have statistically-significant effect sizes in the IL-6 evoked grimace test. FIG. 6 is a graph showing the effect size in the IL-6 evoked grimace test vs. dose of 4ET-01-021. 4ET-01-021 has an ED₅₀ of 0.4 mg/kg.

### Biological Example 9

### In Vivo Determination of eIF4E Phosphorylation in Different Tissues

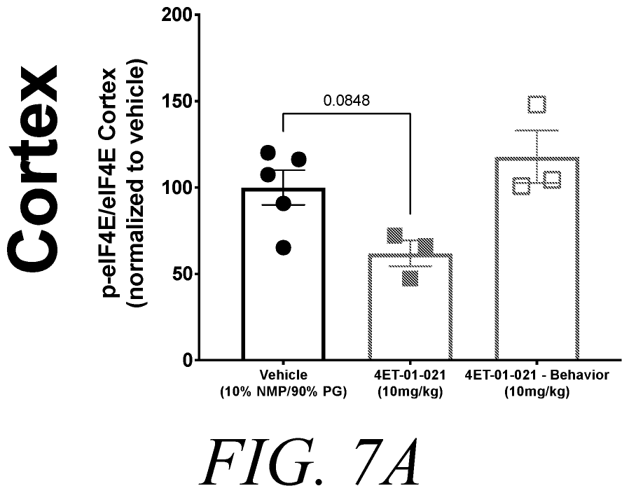

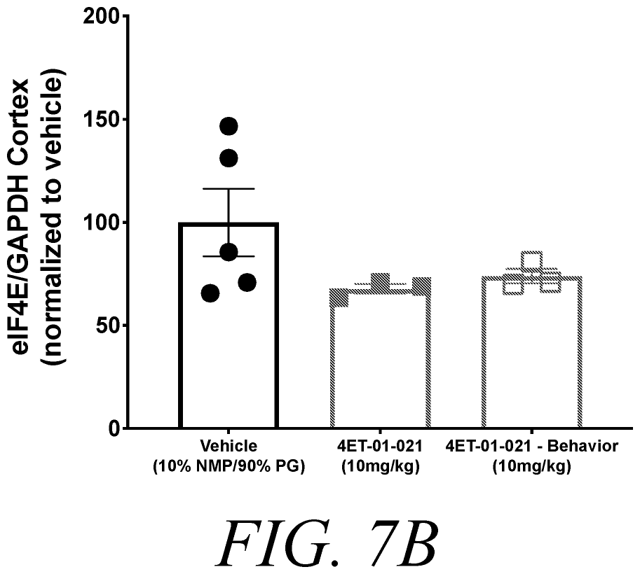

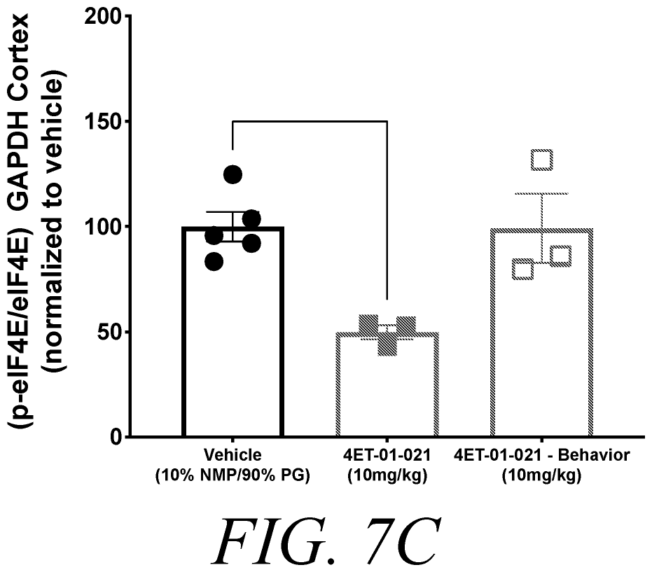

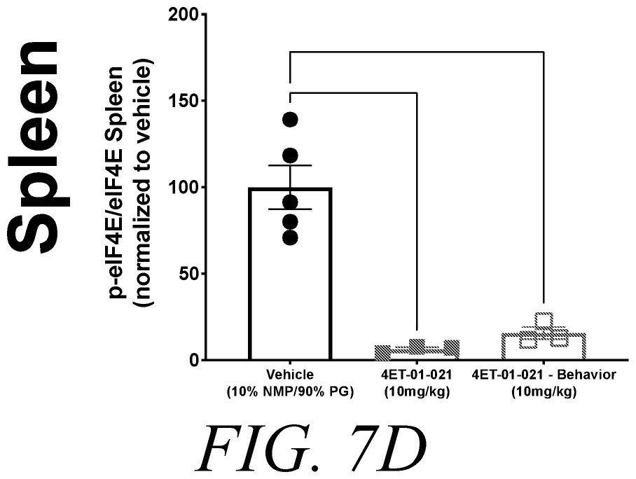

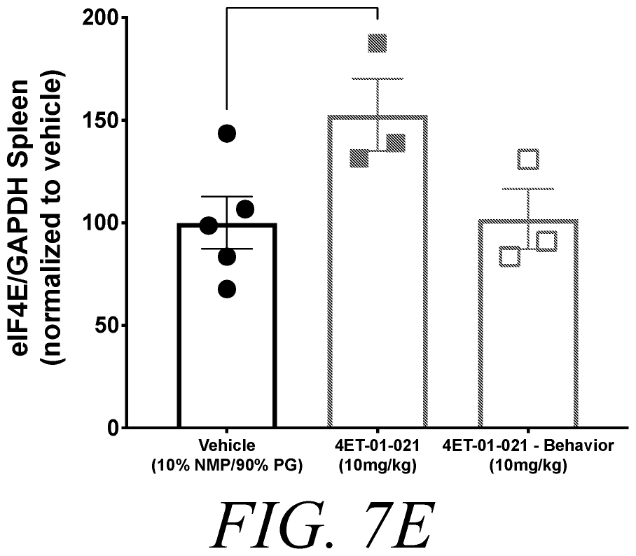

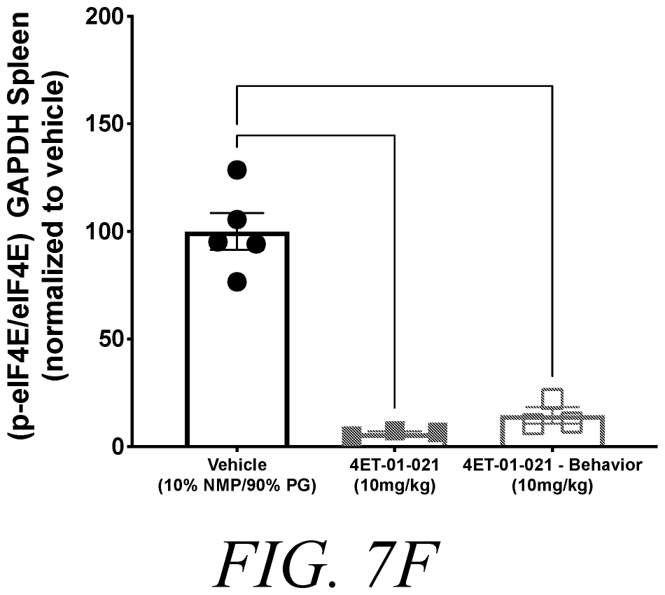

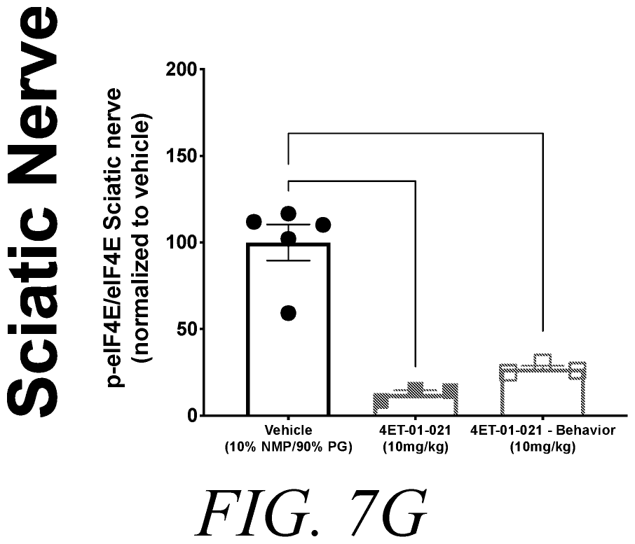

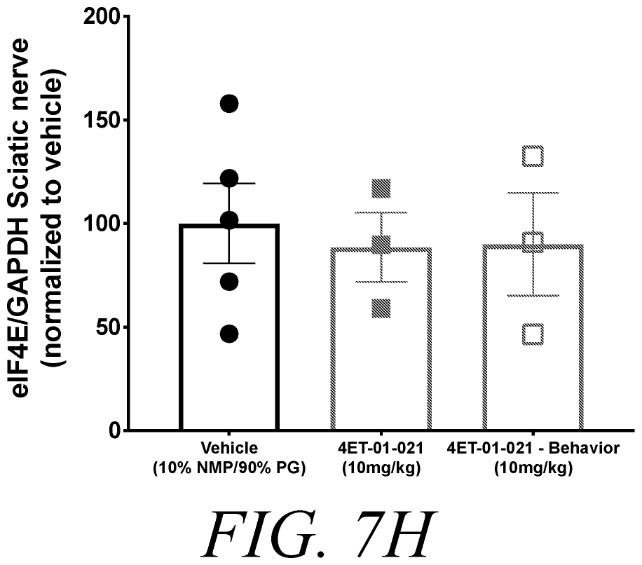

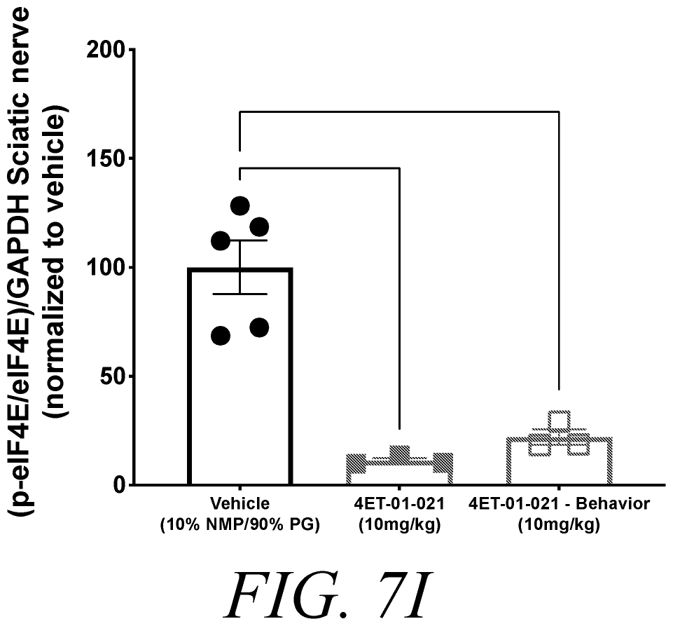

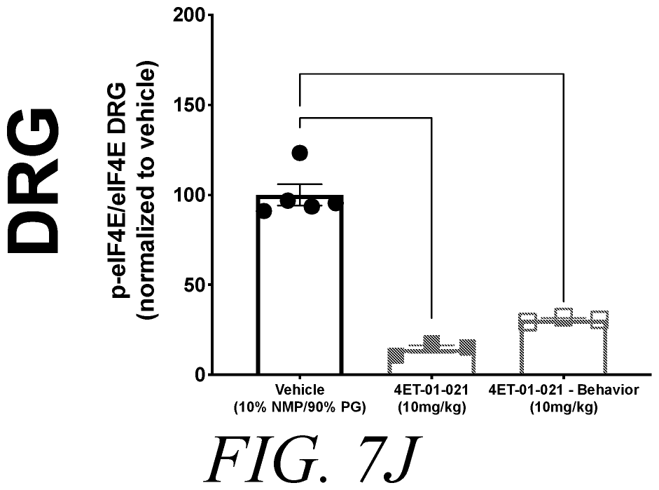

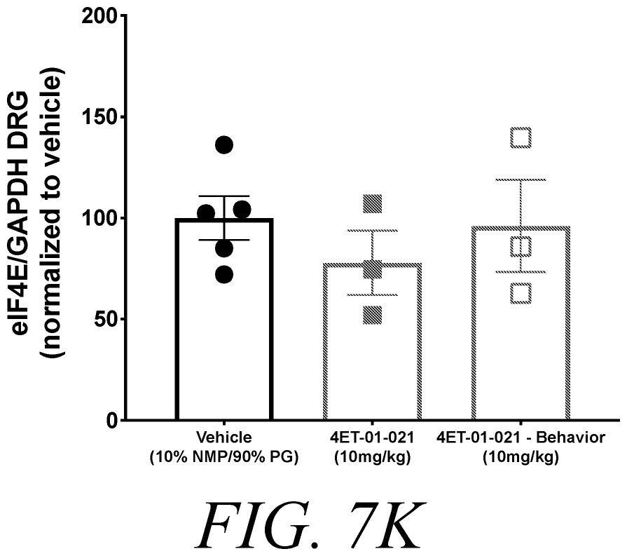

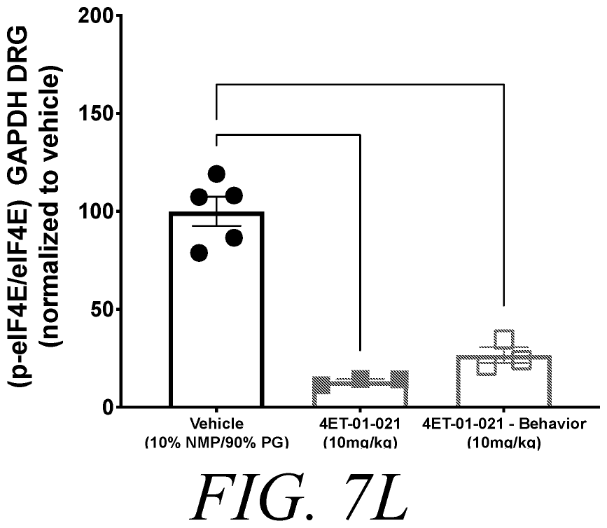

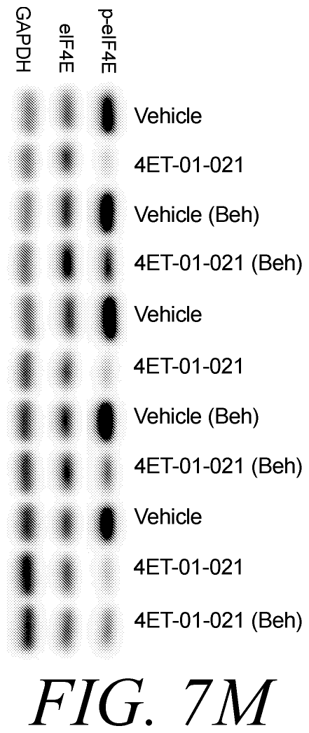

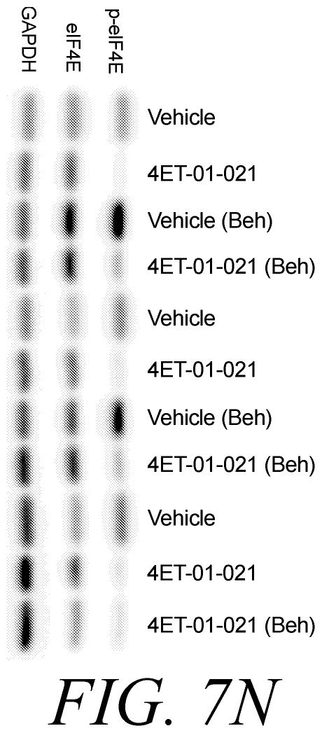

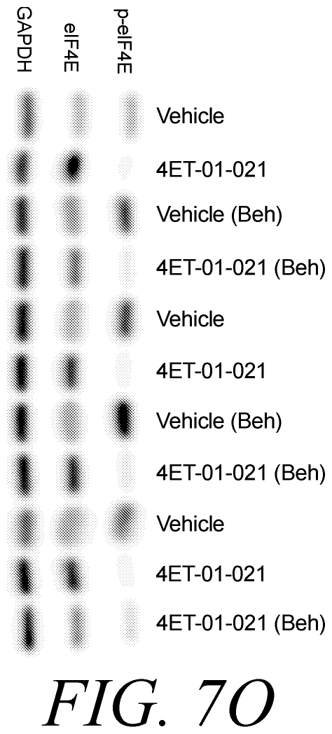

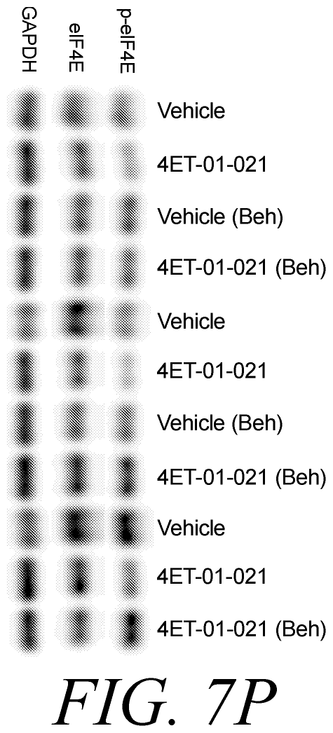

FIGS. 7A-P shows Western blot analysis in tissues from mice dosed with 4ET-01-021. Mice treated with 4ET-01-021 (PO; 10 mg/kg) showed significant inhibition of eIF4E phosphorylation, 2 hours or 4 hours (second treatment group, marked “behavior”, “Beh”) post dose, in DRGs, sciatic nerve, and spleen. However, 4ET-01-021 minimally-inhibited phosphorylation of eIF4E in the brain, as determined by Western blot analysis of brain tissue from the cortex.

1. A compound having the following Structure (II): 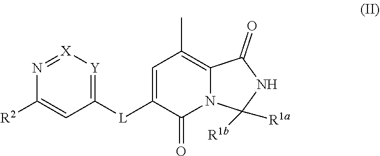
or a pharmaceutically acceptable salt, stereoisomer, tautomer, or prodrug thereof, wherein
R^{1a} is C₁-C₆ alkyl or aryl;
R^{1b} is C₁-C₆ alkyl or aryl,
or R^{1a} and R^{1b}, together with the carbon to which they are both attached, join to form cycloalkyl, cycloalkenyl, heterocyclyl, aryl, or heteroaryl;
R² is —NHR^{3a}, —NHC(═O)R^{3b}, —NHC(═S)R^{3b}, or —C(═O)R^{3c};
R^{3a} is hydrogen, C₁-C₆ alkyl, or C₃-C₆ cycloalkyl, each of which is optionally substituted with one or more substituents selected from the group consisting of hydroxyl, C₃-C₆ cycloalkyl, —NHS(O)₂CH₃, heterocyclyl, —C(═O)OH, —C(═O)N(R^{3d})R^{3d}, or —N(R^{3d})R^{3d};
R^{3b} is C₁-C₆ alkyl, C₃-C₆ cycloalkyl, or heterocyclyl each of which is optionally substituted with one or more substituents selected from the group consisting of hydroxyl, halo, C₁-C₆ alkyl, C₃-C₆ cycloalkyl, —NHS(O)₂CH₃, —N(R^{3d})R^{3d}, heterocyclyl, —C(═O)OH, —C(═O)N(R^{3d})R^{3d}, —NHC(═O)CH₃, —CH₂C(═O)OH,
R^{3c} is —N(R^{3d})R^{3d} or heterocyclyl;
R^{3d} is, at each occurrence, independently hydrogen, C₁-C₆ alkyl, or C₃-C₆ cycloalkyl;
L is —NH— or —CH₂NH—; and
X is N and Y is CH or X is CH and Y is N,
provided that:
when R^{1a} and R^{1b} are both —CH₃ or when R^{1a} and R^{1b} join to form a 5- or 6-membered cycloalkyl or heterocyclyl, then R² does not have the following structure:
—NH₂ or

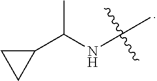 [SMILES: *NC(C)C1CC1]

2-4. (canceled)

5. The compound of claim 1, wherein R^{1a} is methyl or phenyl.

6. (canceled)

7. The compound of claim 1, wherein R^{1b} is methyl.

8. The compound of claim 1, wherein R^{1a} and R^{1b}, together with the carbon to which they are both attached, join to form cyclopentyl or cyclohexyl.

9. (canceled)

10. The compound of claim 1, wherein R^{1a} and R^{1b}, together with the carbon to which they are both attached, join to form cyclopentenyl, cyclohexenyl, or cycloheptenyl.

11. (canceled)

12. The compound of claim 1, wherein R^{1a} and R^{1b}, together with the carbon to which they are both attached, join to form heterocyclyl, aryl, or heteroaryl.

13-14. (canceled)

15. The compound of claim 1, wherein the compound has one of the following structures: 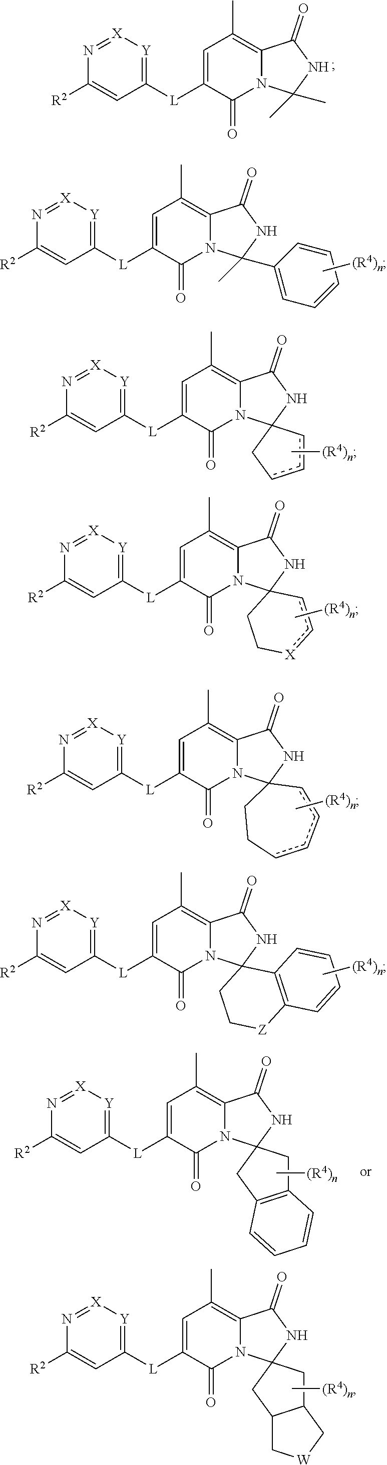
or a pharmaceutically acceptable salt, stereoisomer, tautomer, or prodrug thereof, wherein [symbol]
indicates a double or single bond;
R⁴ is, at each occurrence, independently C₁-C₆ alkyl, C₃-C₆ cycloalkyl, halo, haloalkyl, hydroxyl, —NHS(O)₂CH₃, or —C(O)OH,
or two R⁴, together with the carbon to which they are both attached, join to form a cycloalkyl;
W is N or O;
Z is C or O; and
n is 0, 1, 2, 3, or 4.

16. The compound of claim 15, wherein n is 0, 1, or 2.

17-24. (canceled)

25. The compound of claim 1, wherein R² has one of the following structures:

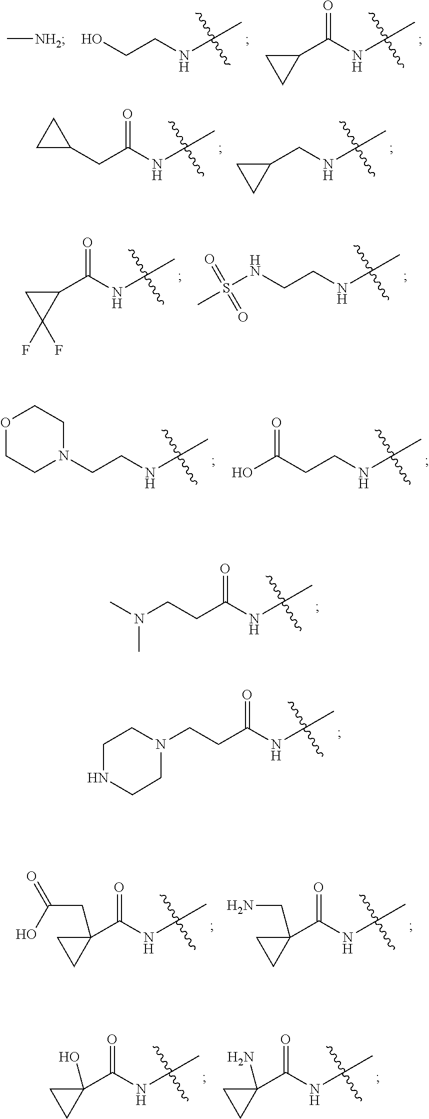

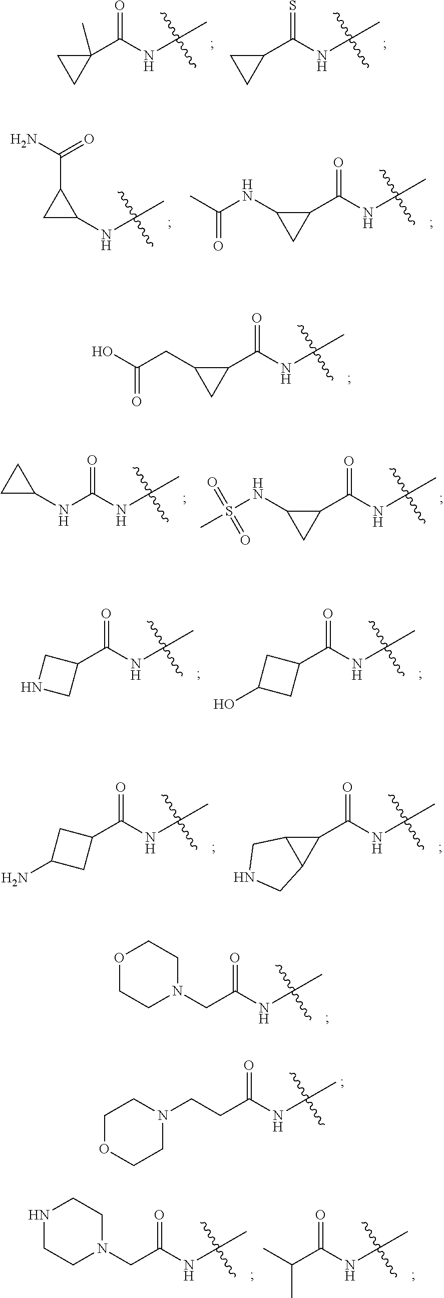

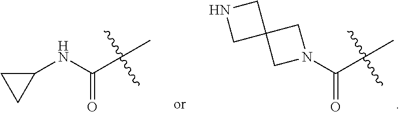 [SMILES: *C(=O)NC1CC1 ; *C(=O)N1CC2(CNC2)C1]

26. The compound of claim 1, wherein R² has one of the following structures:
—NH₂ or

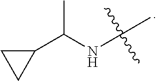 [SMILES: *NC(C)C1CC1]

27. The compound of claim 1, wherein X is CH and Y is N.

28. The compound of claim 1, wherein X is N and Y is CH.

29. The compound of claim 1, wherein L is —NH—.

30. The compound of claim 1, wherein L is —CH₂NH—.

31. The compound of claim 1, wherein the compound has one of the following structures: 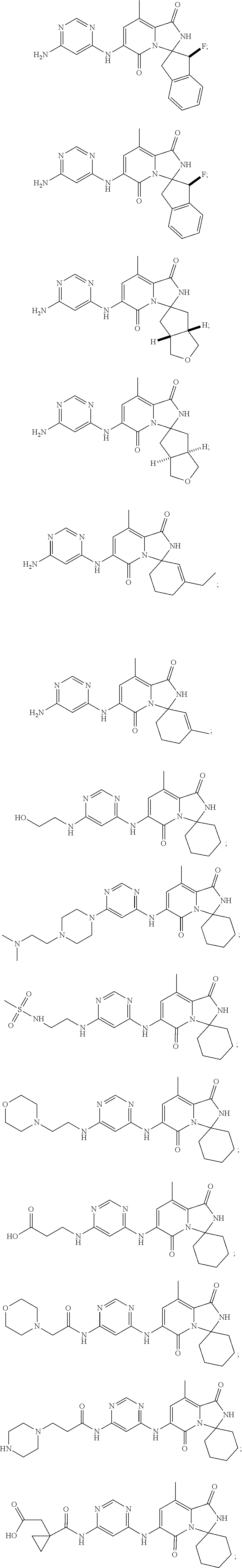 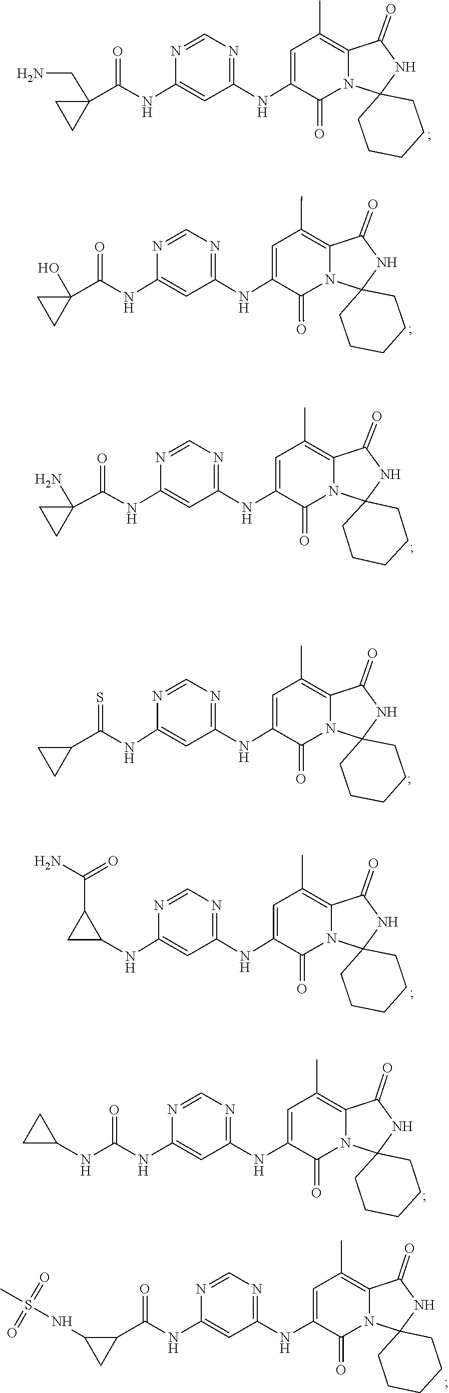 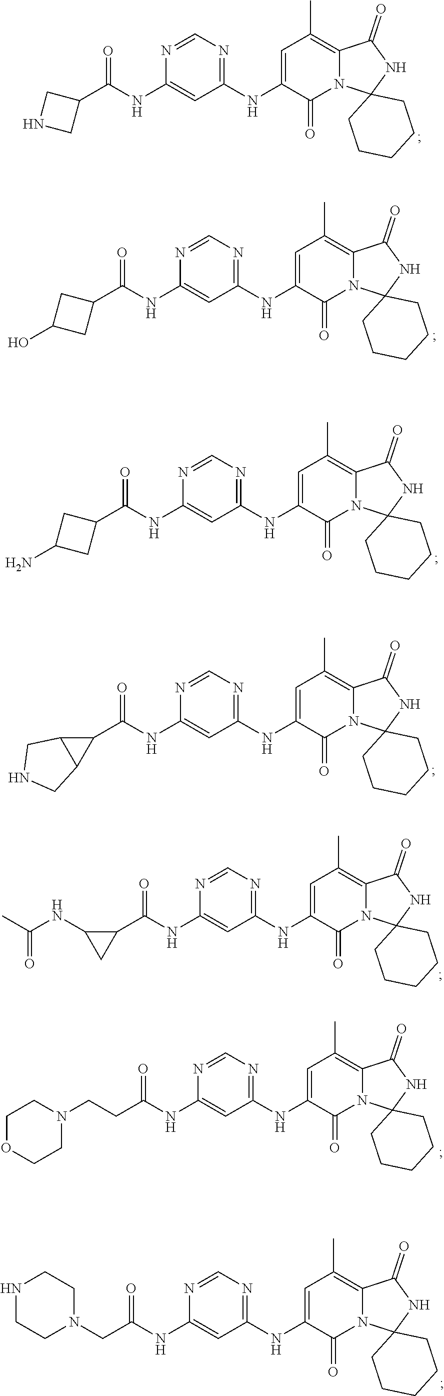 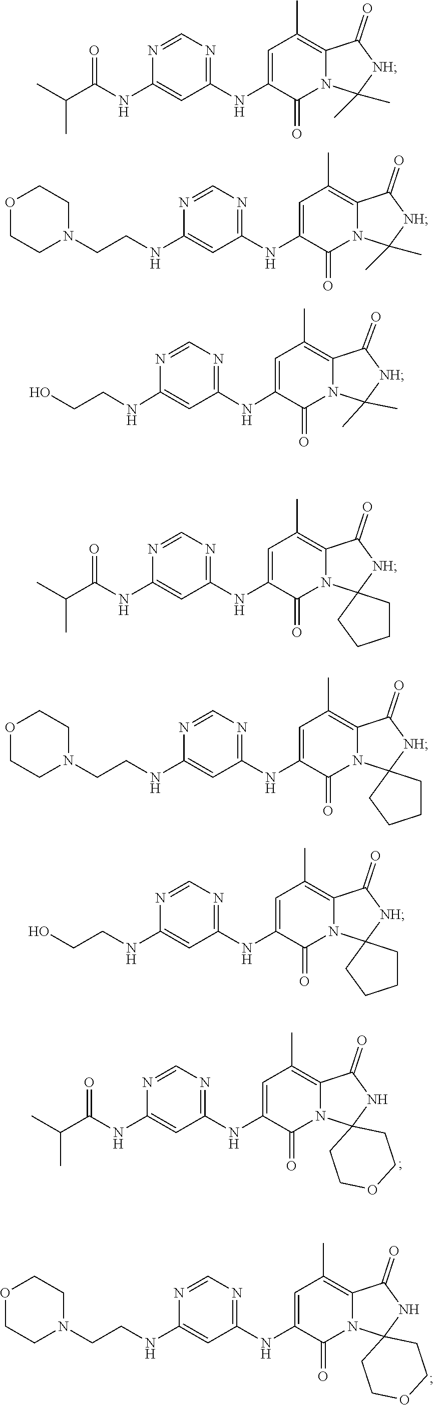 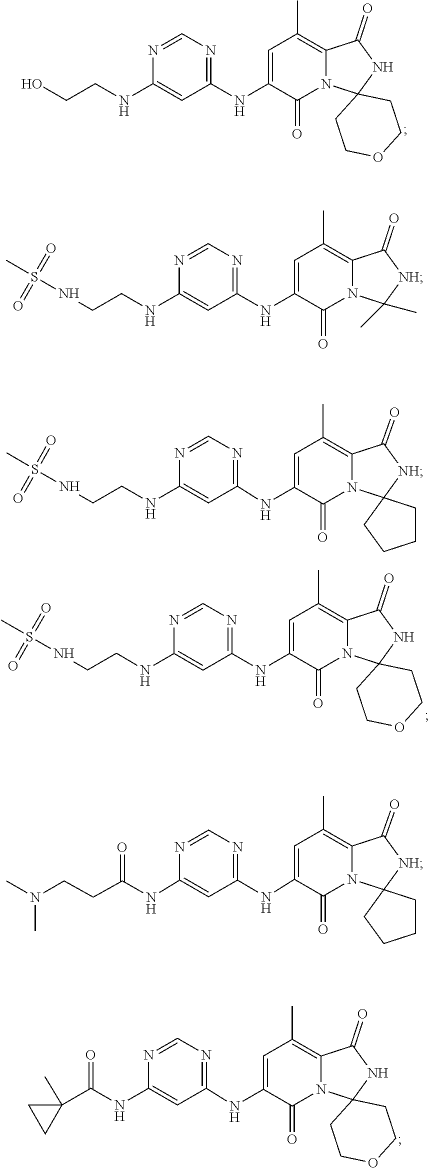 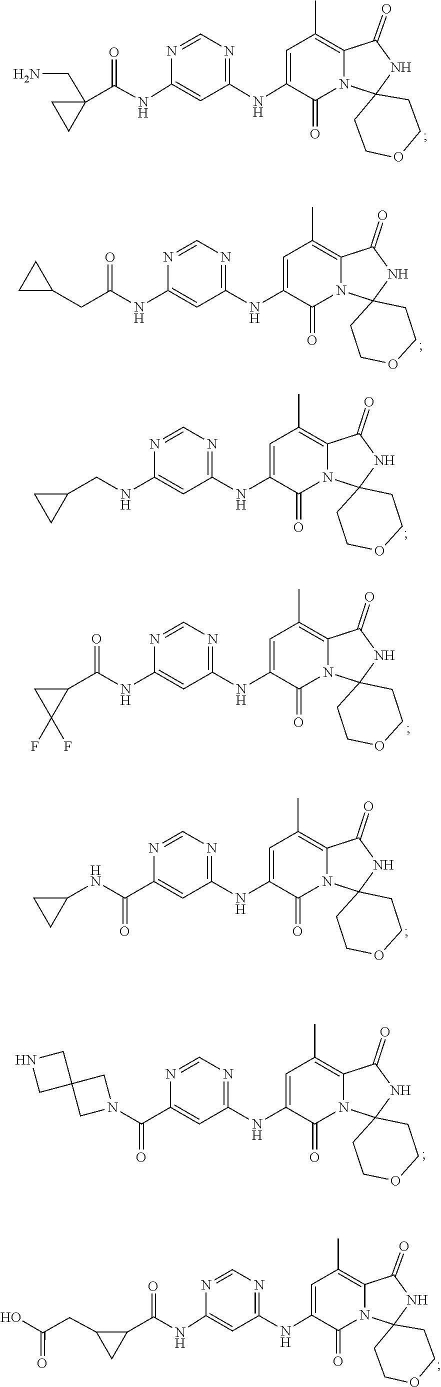 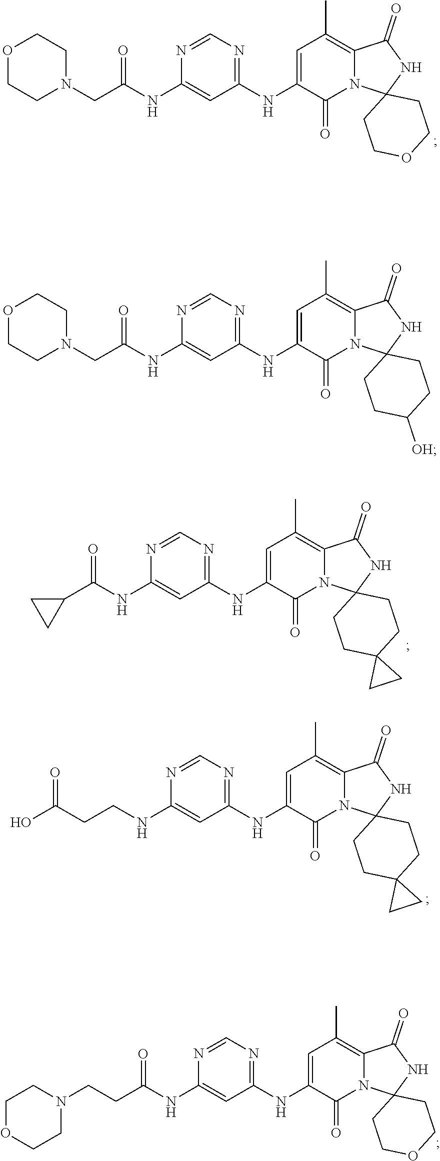 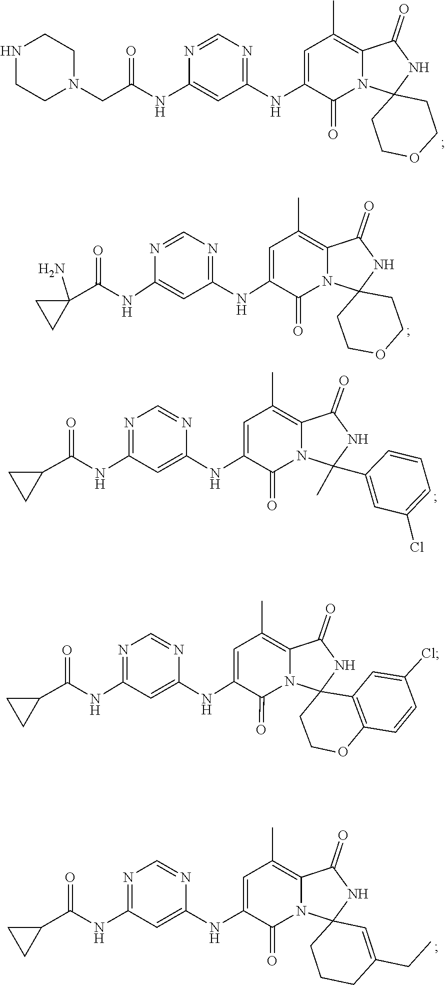 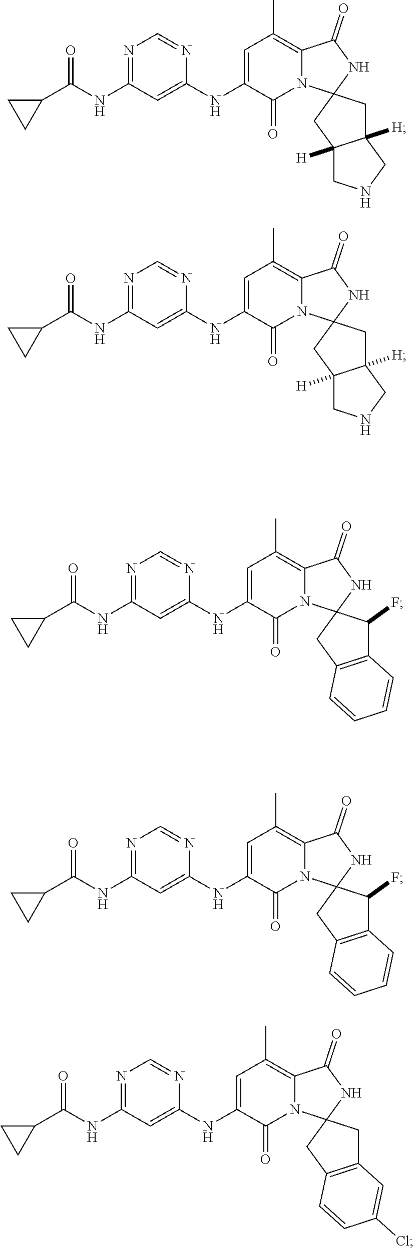 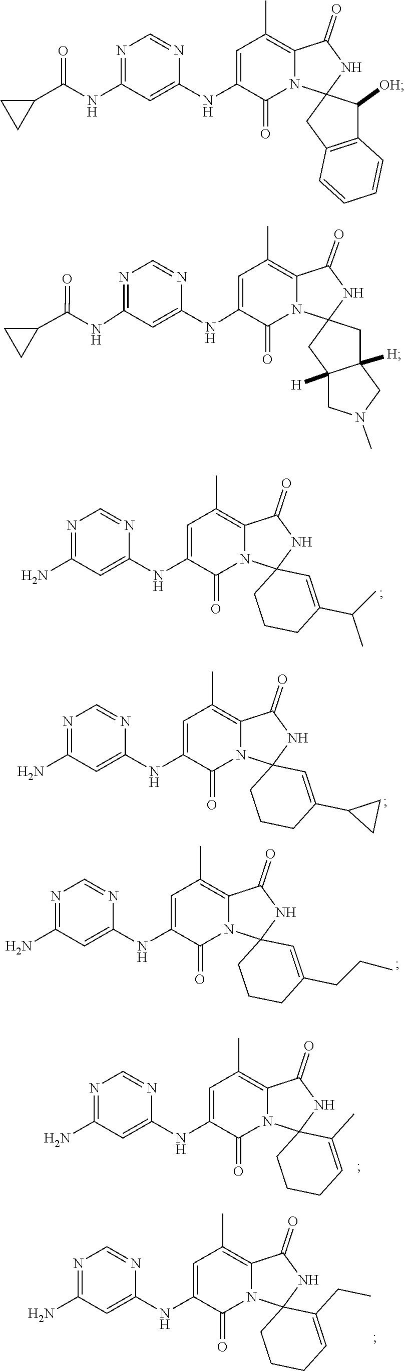 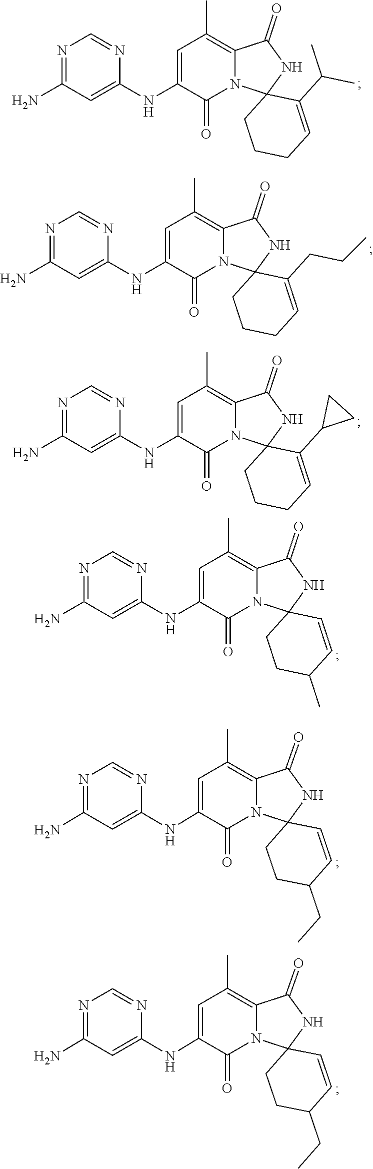 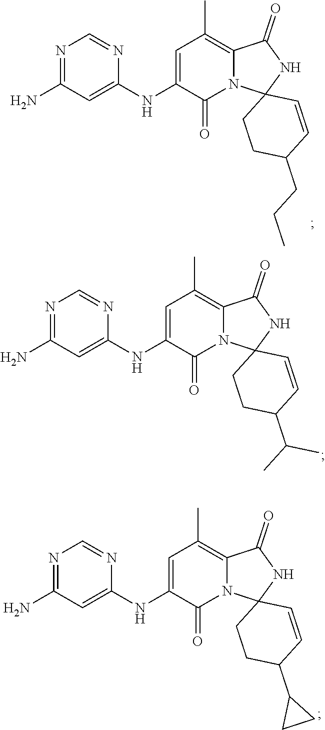 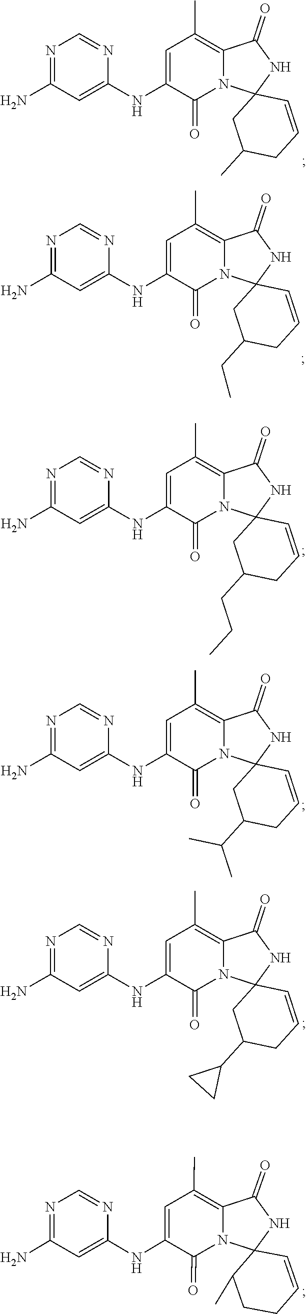 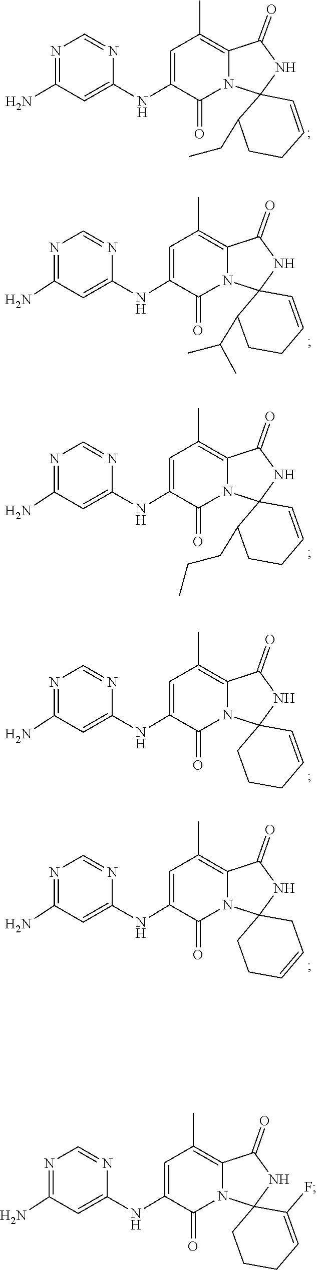 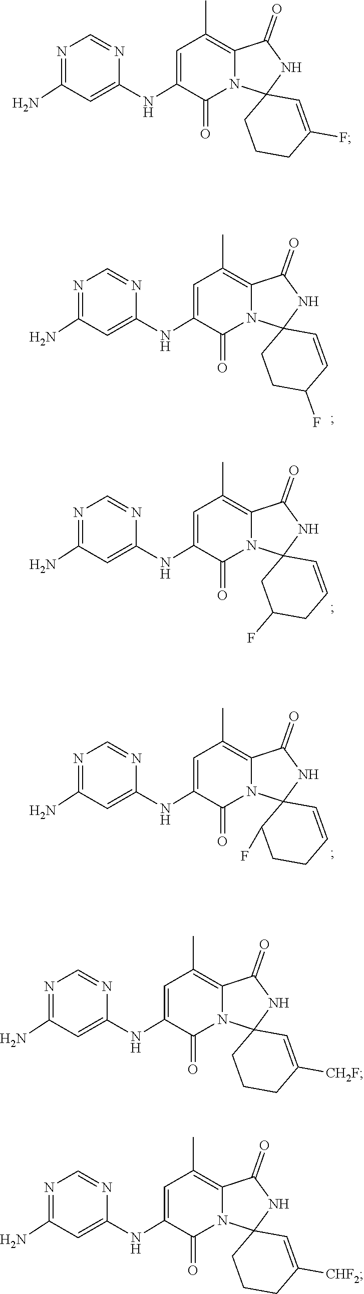 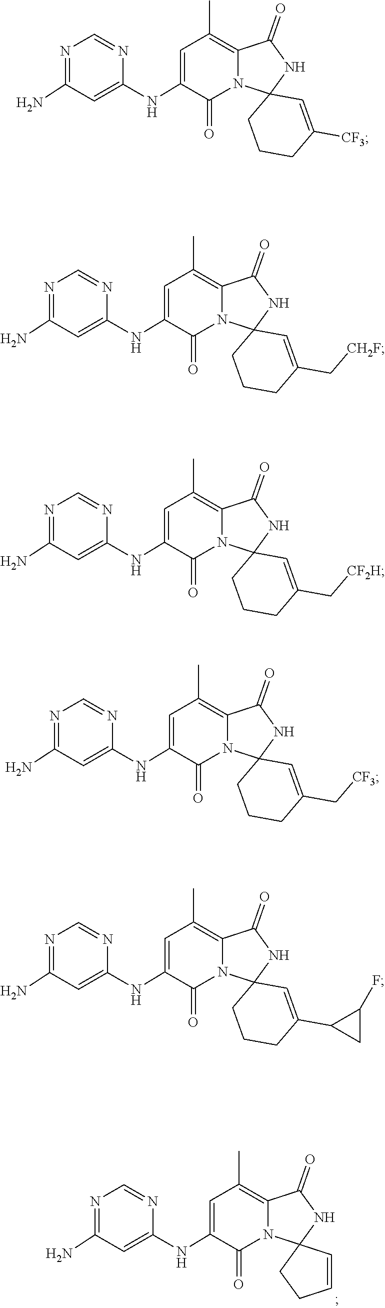 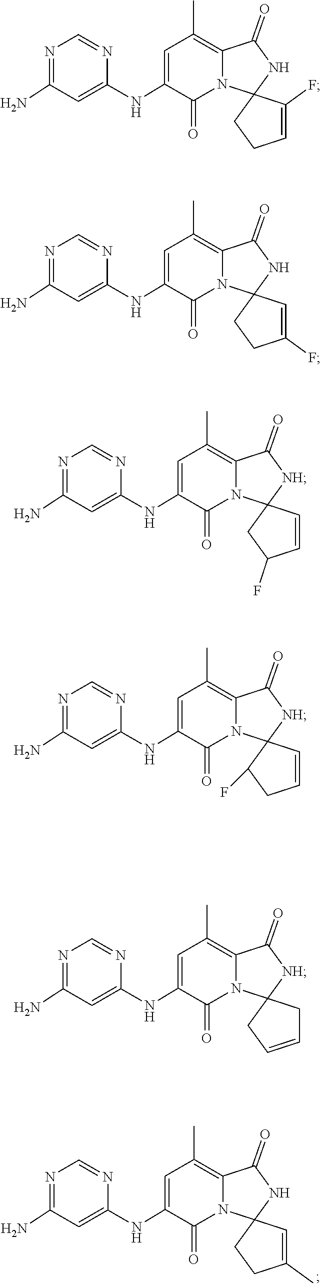 [SMILES: Cc1cc(Nc2cc(N)ncn2)c(=O)n2c1C(=O)NC21CCC=C1F ; Cc1cc(Nc2cc(N)ncn2)c(=O)n2c1C(=O)NC21C=C(F)CC1 ; Cc1cc(Nc2cc(N)ncn2)c(=O)n2c1C(=O)NC21C=CC(F)C1 ; Cc1cc(Nc2cc(N)ncn2)c(=O)n2c1C(=O)NC21C=CCC1F ; Cc1cc(Nc2cc(N)ncn2)c(=O)n2c1C(=O)NC21CC=CC1 ; CC1=CC2(CC1)NC(=O)c1c(C)cc(Nc3cc(N)ncn3)c(=O)n12] 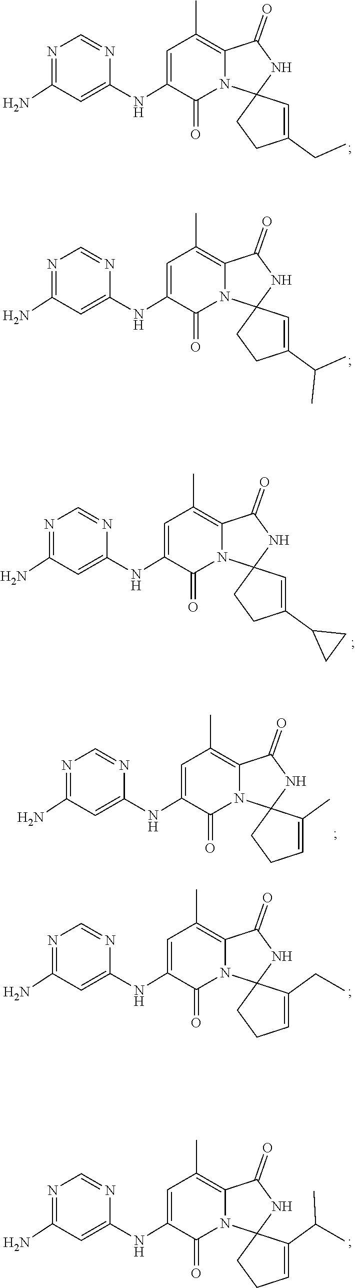 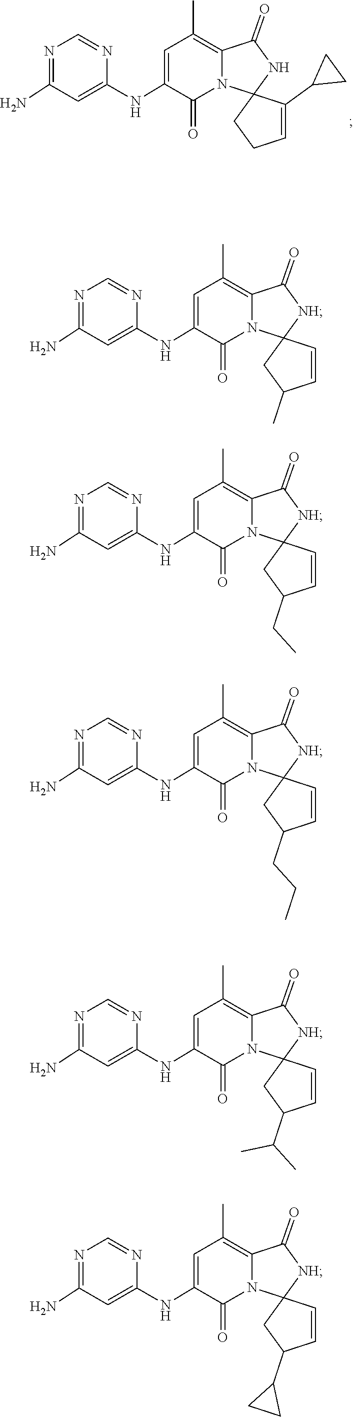 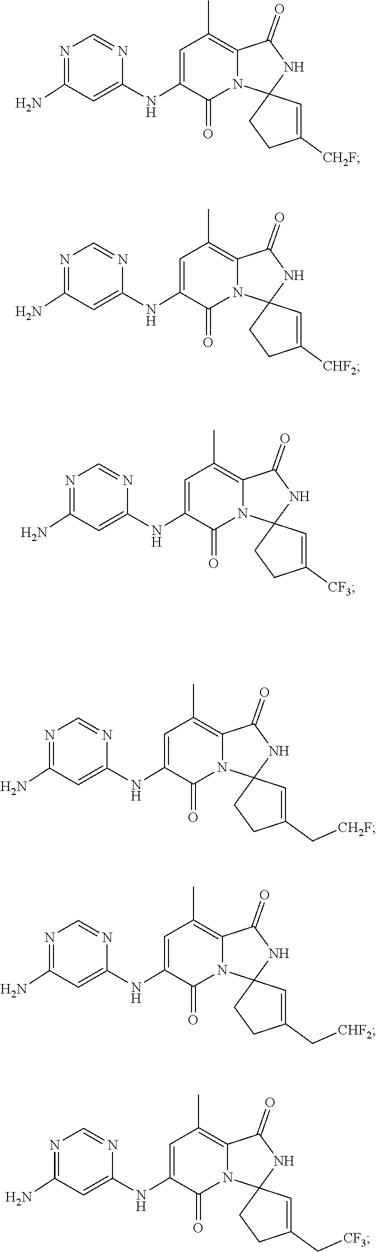 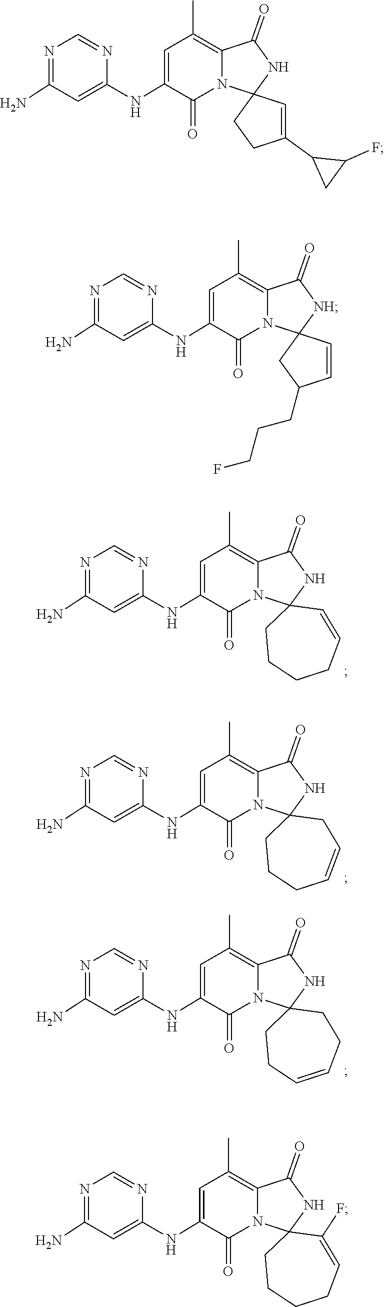 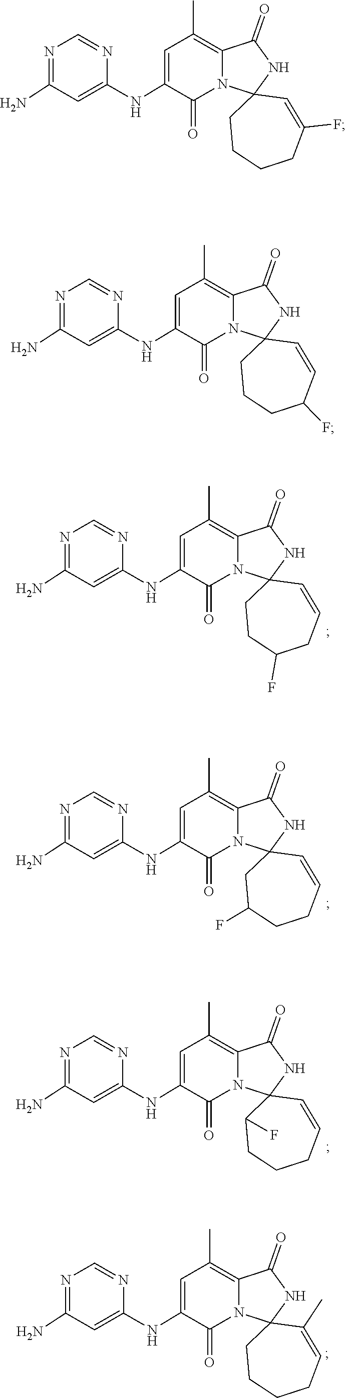 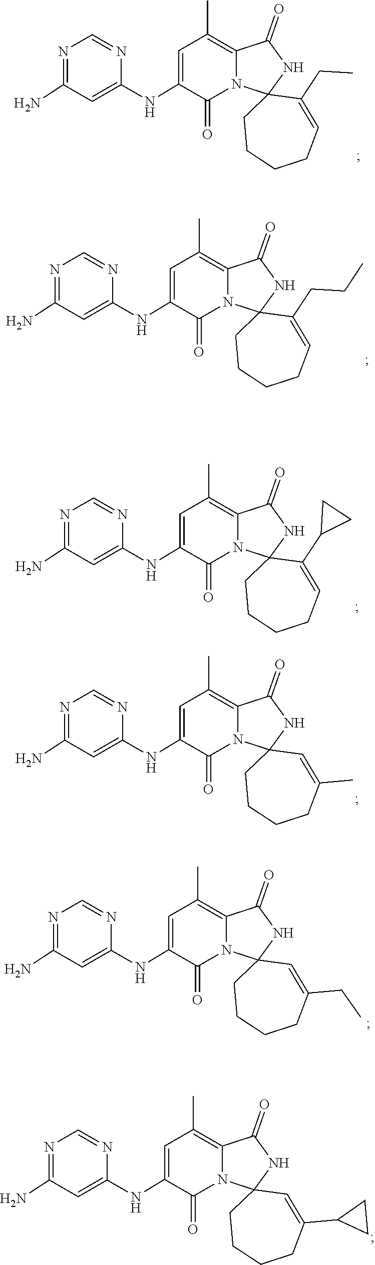 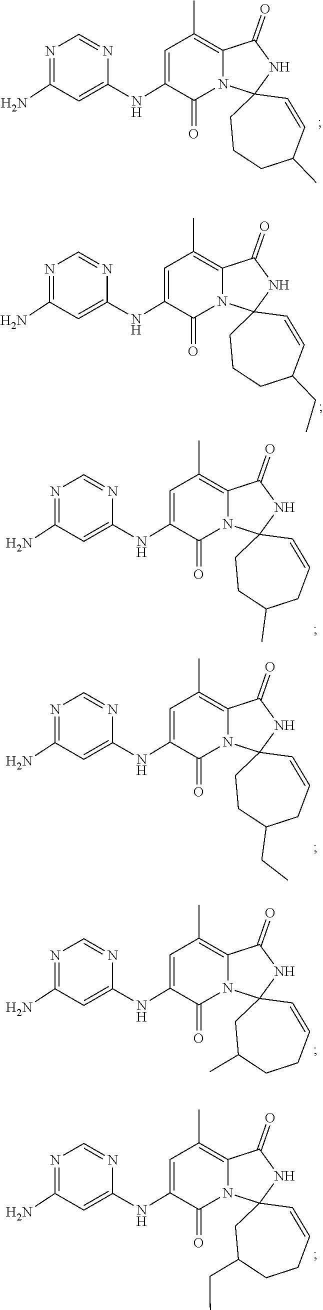 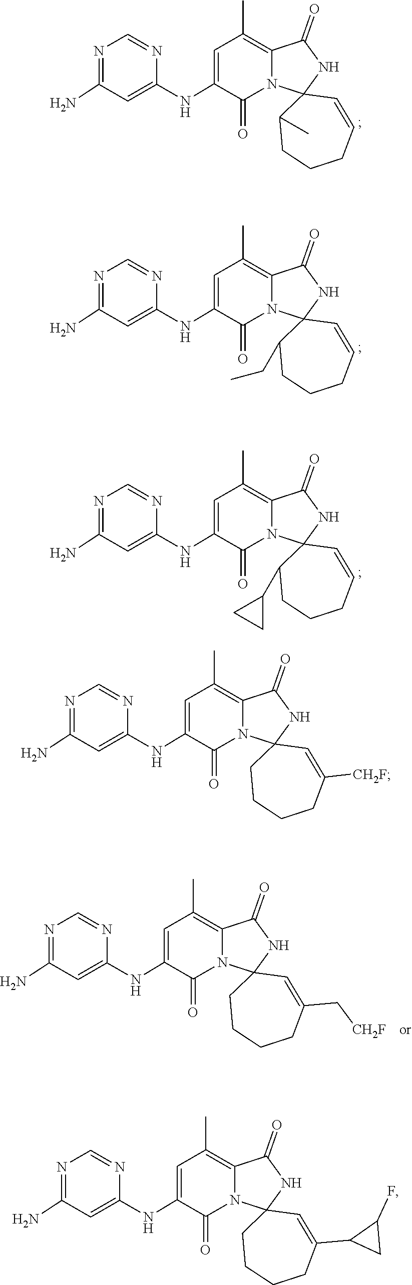
or a pharmaceutically acceptable salt, stereoisomer, tautomer, or prodrug thereof.

32. A pharmaceutical composition comprising the compound of claim 1, and a pharmaceutically acceptable carrier, diluent or excipient.

33. A method of treating a disease or disorder, comprising administering a therapeutically effective amount of a compound of claim 1, to a subject in need thereof.

34. (canceled)

35. The method of claim 33, wherein the disease or disorder is Huntington's disease, Alzheimer's, high fat induced obesity, Fragile X Symdrome, lupus, Covid19 related acute respiratory distress syndrome (ARDS), non-alcoholic fatty liver disease (NAFLD), or viral induced pain.

36. A method for treating neuropathic pain, the method comprising administering a therapeutically effective amount of a compound having the following Structure (II): 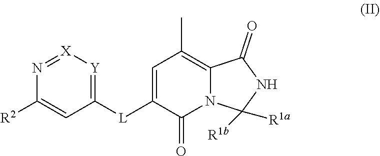
or a pharmaceutically acceptable salt, stereoisomer, tautomer, or prodrug thereof, wherein
R^{1a} is C₁-C₆ alkyl or aryl;
R^{1b} is C₁-C₆ alkyl or aryl,
or R^{1a} and R^{1b}, together with the carbon to which they are both attached, join to form cycloalkyl, cycloalkenyl, heterocyclyl, aryl, or heteroaryl;
R² is heterocyclyl, —NHR^{3a}, —NHC(═O)R^{3b}, —NHC(═S)R^{3b}, or —C(═O)R^{3c};
R^{3a} is hydrogen, C₁-C₆ alkyl, or C₃-C₆ cycloalkyl, each of which is optionally substituted with one or more substituents selected from the group consisting of hydroxyl, C₃-C₆ cycloalkyl, —NHS(O)₂CH₃, heterocyclyl, —C(═O)OH, —C(═O)N(R^{3d})R^{3d}, or —N(R^{3d})R^{3d};
R^{3b} is C₁-C₆ alkyl, C₃-C₆ cycloalkyl, or heterocyclyl each of which is optionally substituted with one or more substituents selected from the group consisting of hydroxyl, halo, C₁-C₆ alkyl, C₃-C₆ cycloalkyl, —NHS(O)₂CH₃, —N(R^{3d})R^{3d}, heterocyclyl, —C(═O)OH, —C(═O)N(R^{3d})R^{3d}, —NHC(═O)CH₃, —CH₂C(═O)OH,
R^{3c} is —N(R^{3d})R^{3d} or heterocyclyl;
R^{3d} is, at each occurrence, independently hydrogen, C₁-C₆ alkyl, or C₃-C₆ cycloalkyl;
L is —NH— or —CH₂NH—; and
X is N and Y is CH or X is CH and Y is N, to a subject in need thereof.

37. A method for treating neuropathic pain, the method comprising administering a therapeutically effective amount of a compound having one of the following structures: 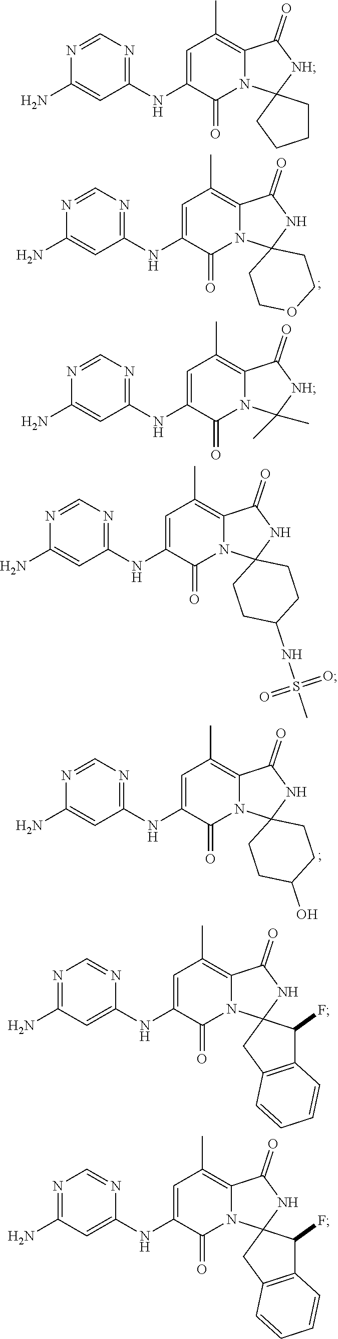 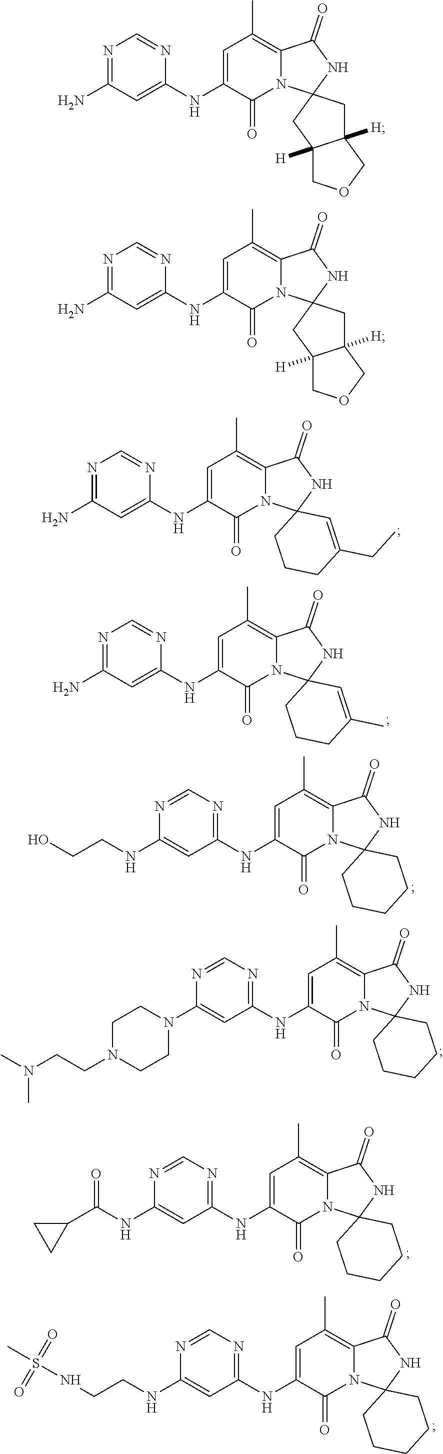 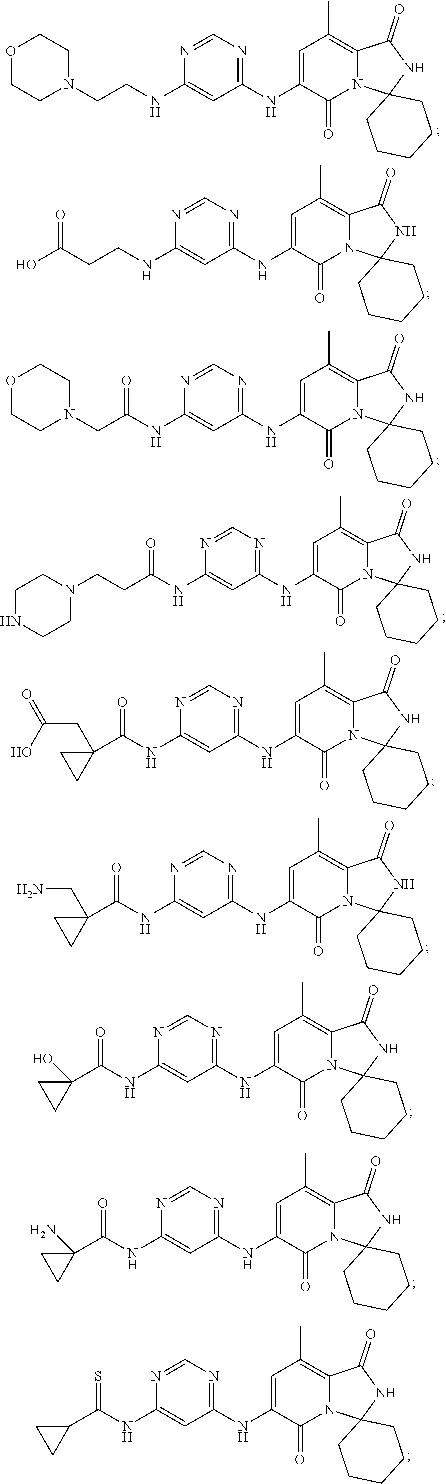 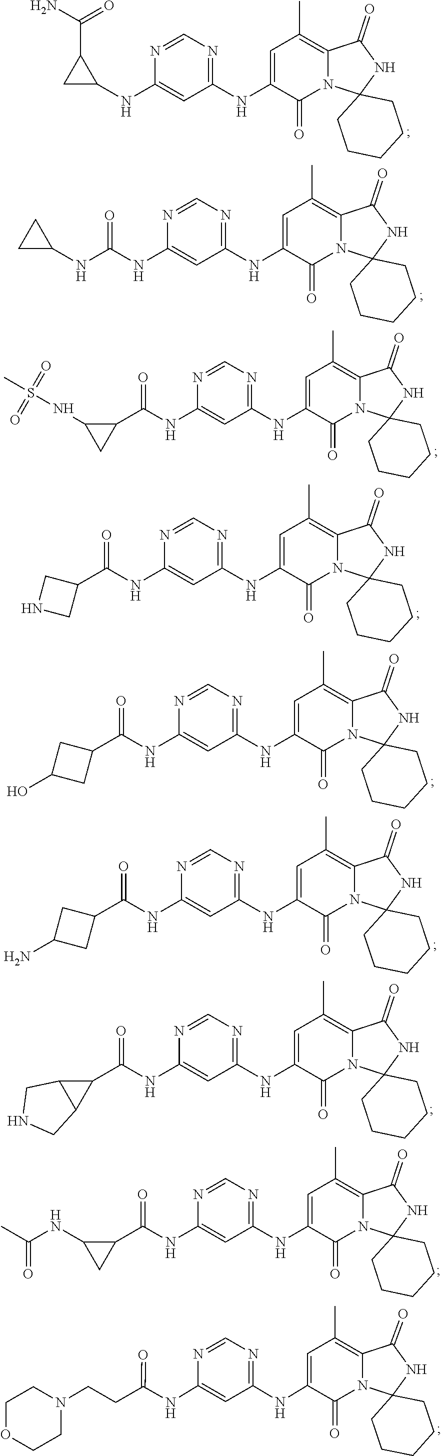 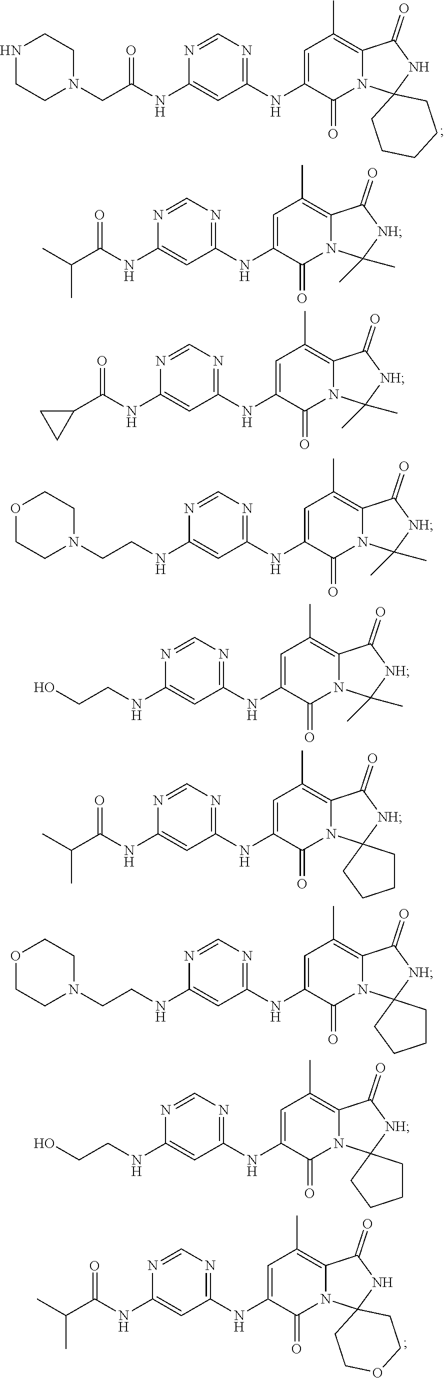 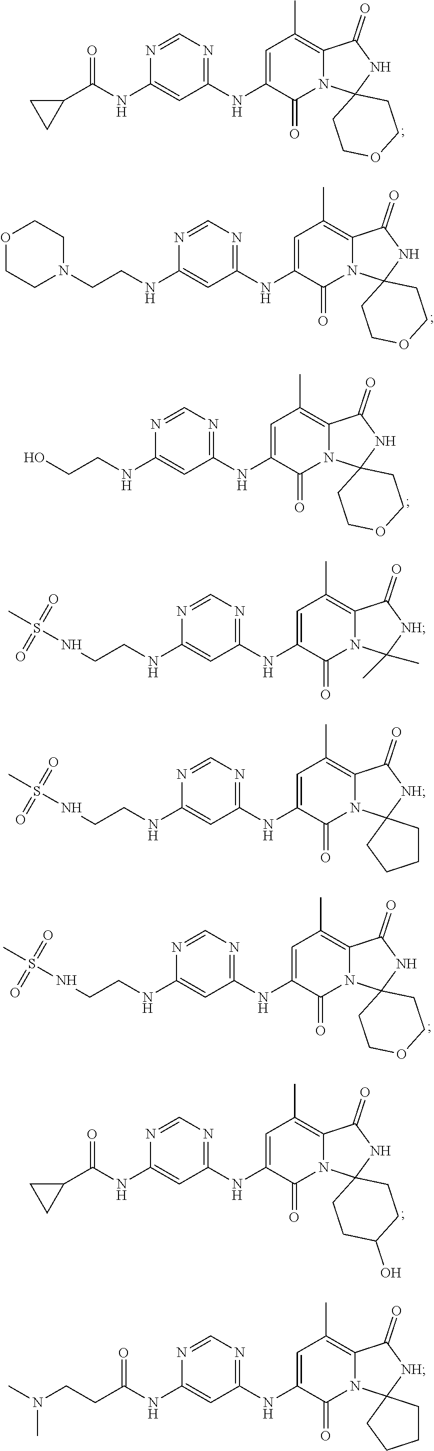 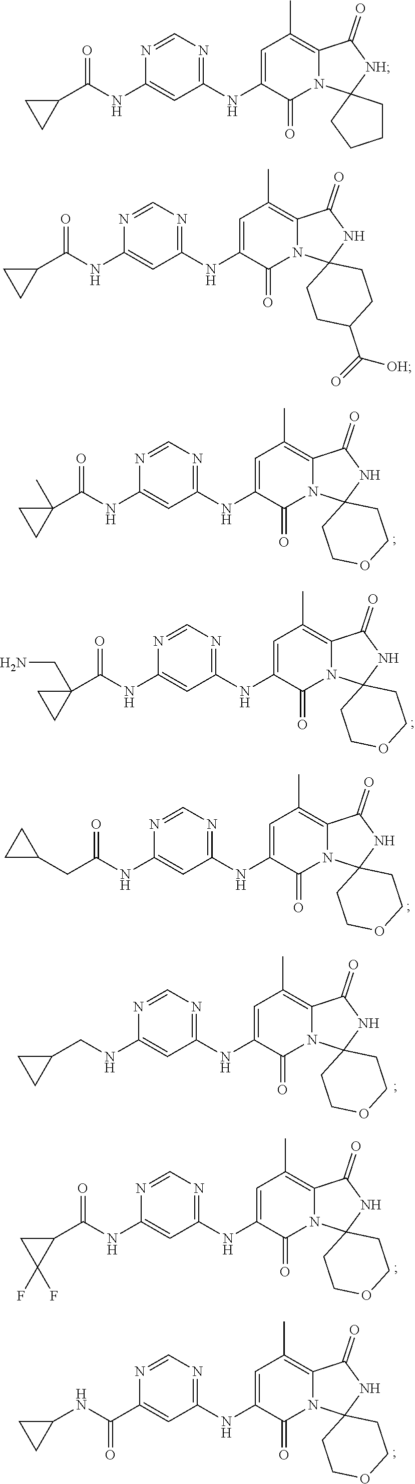 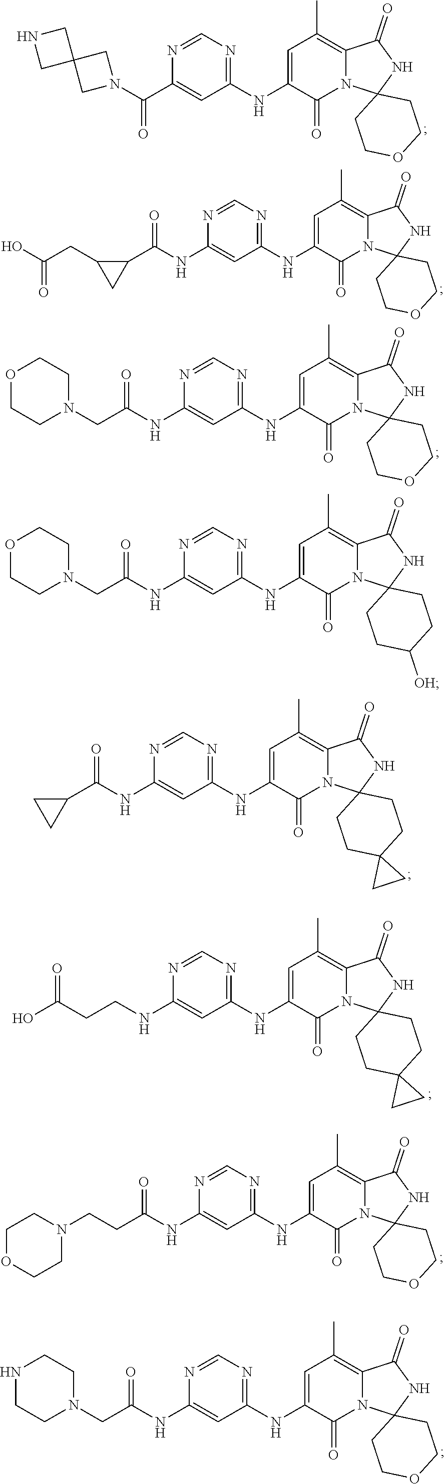 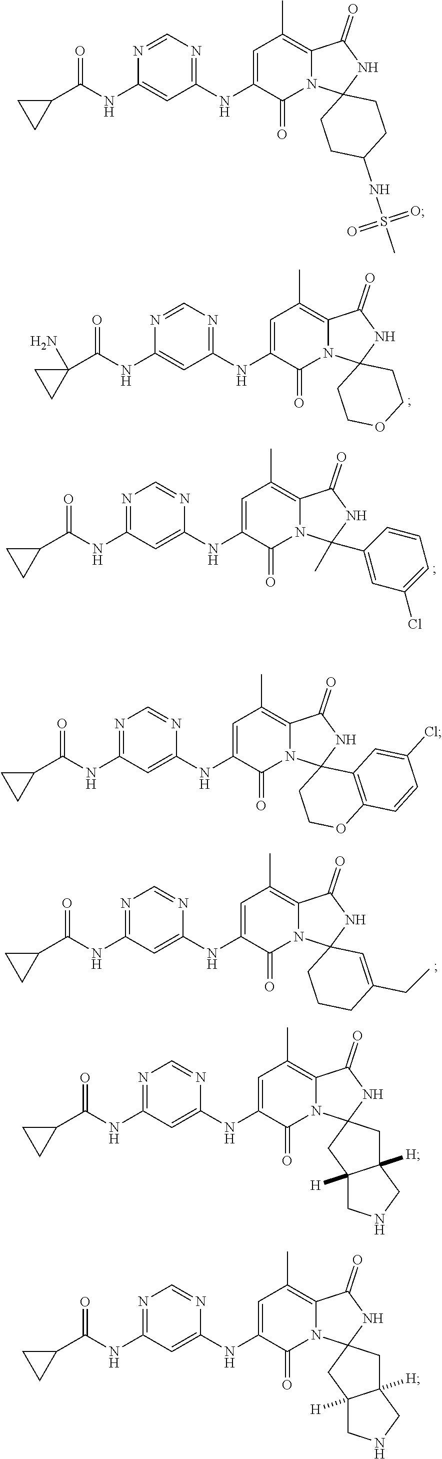 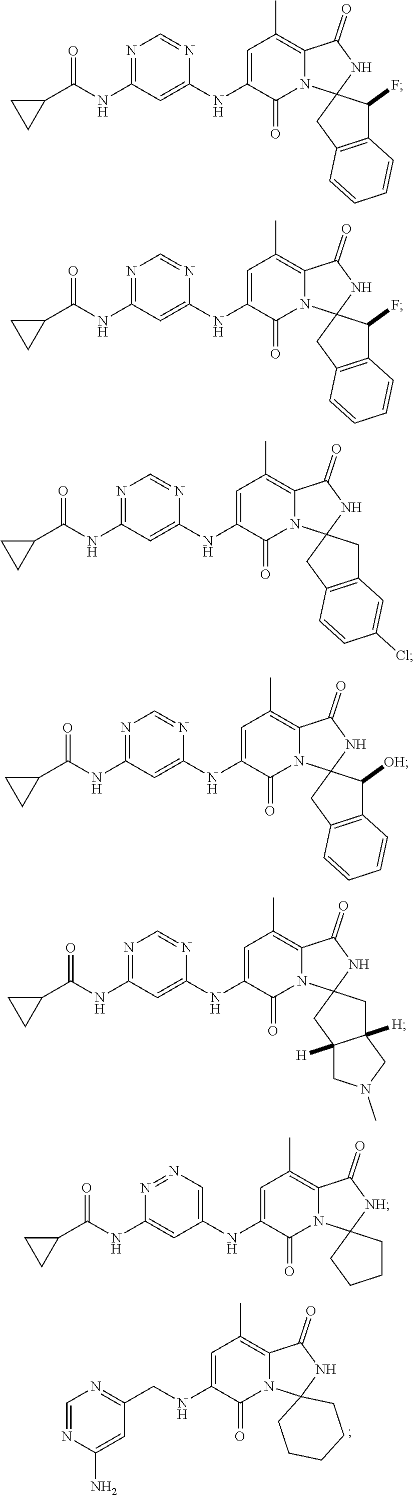 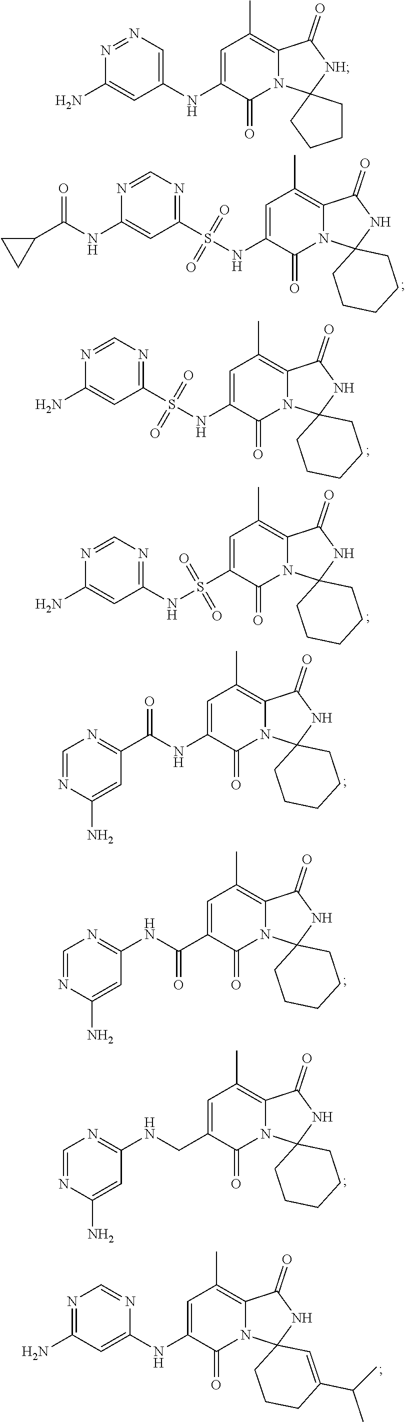 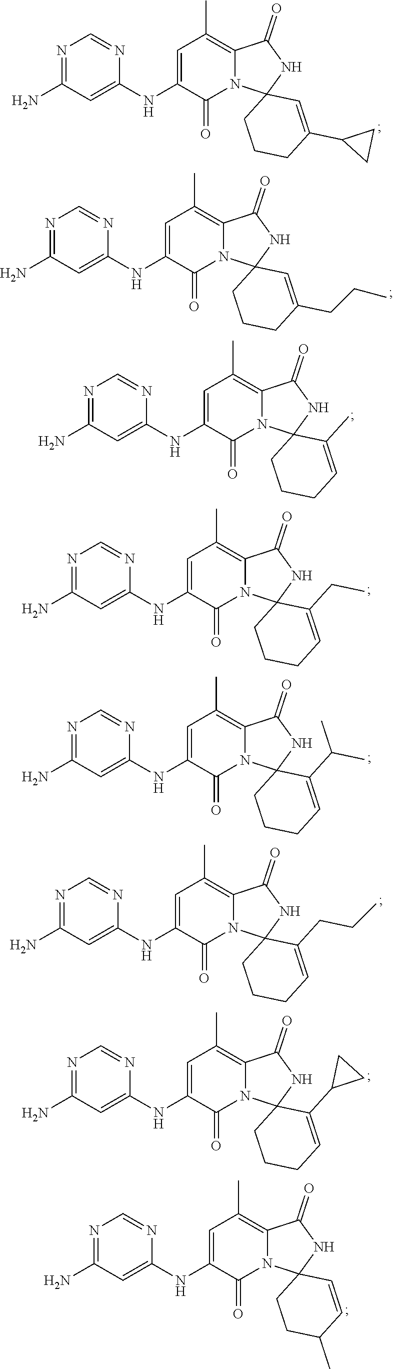 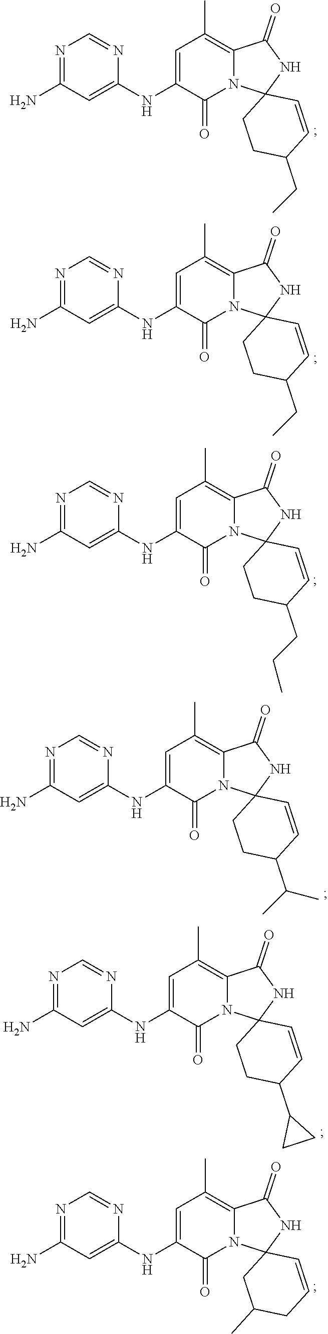 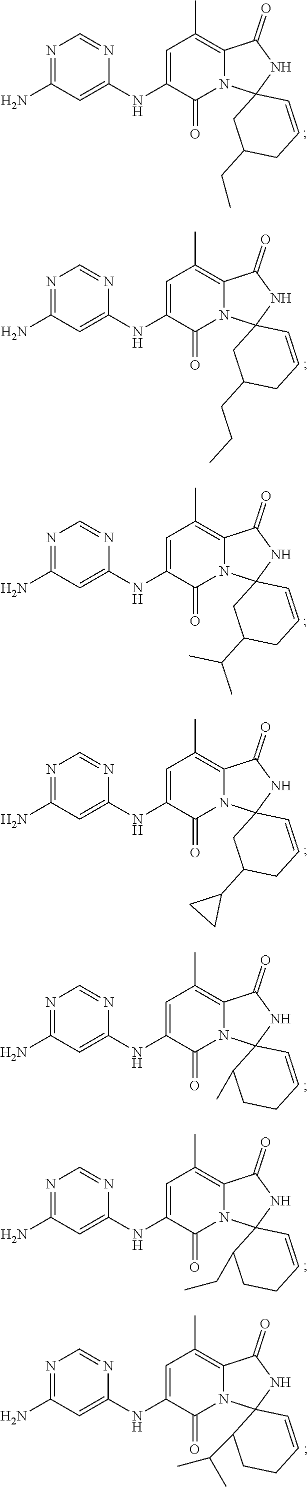 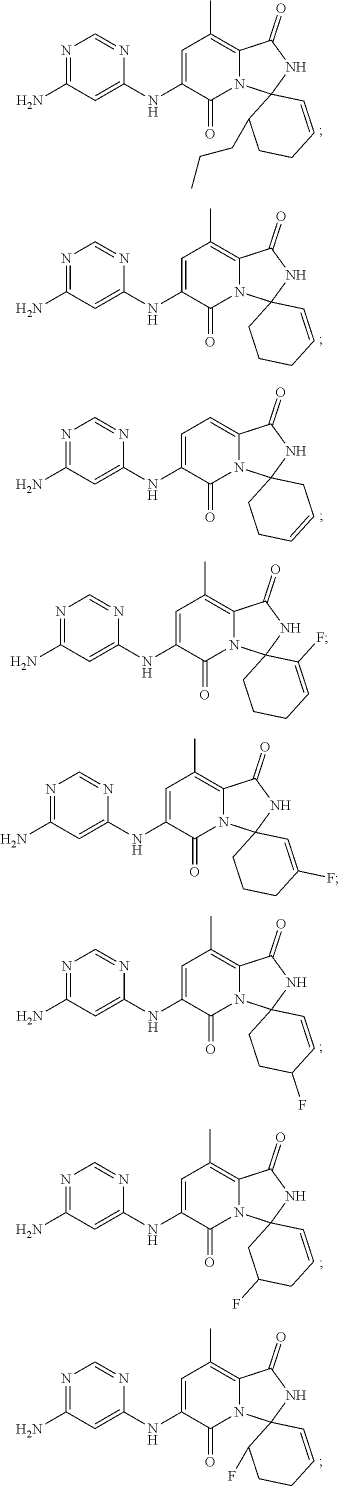 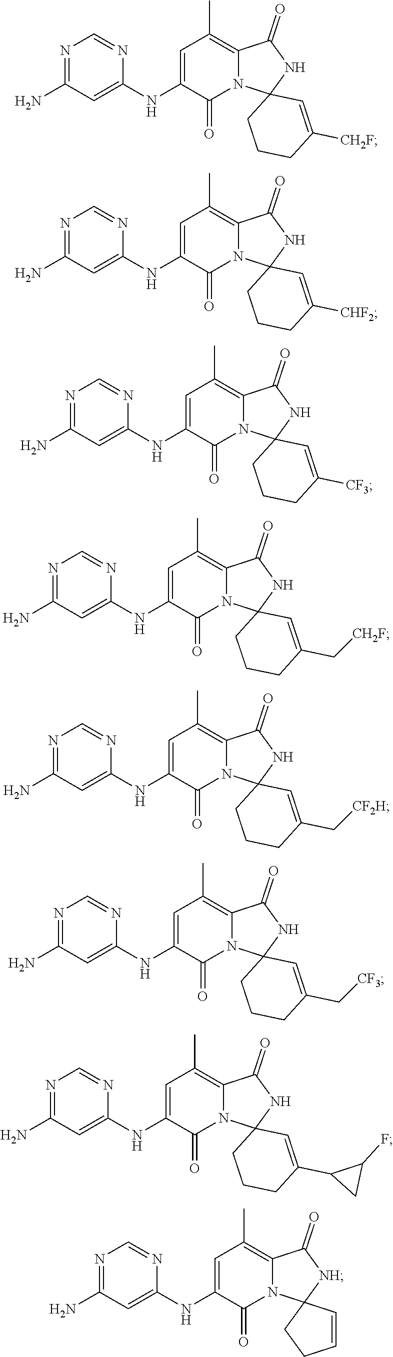 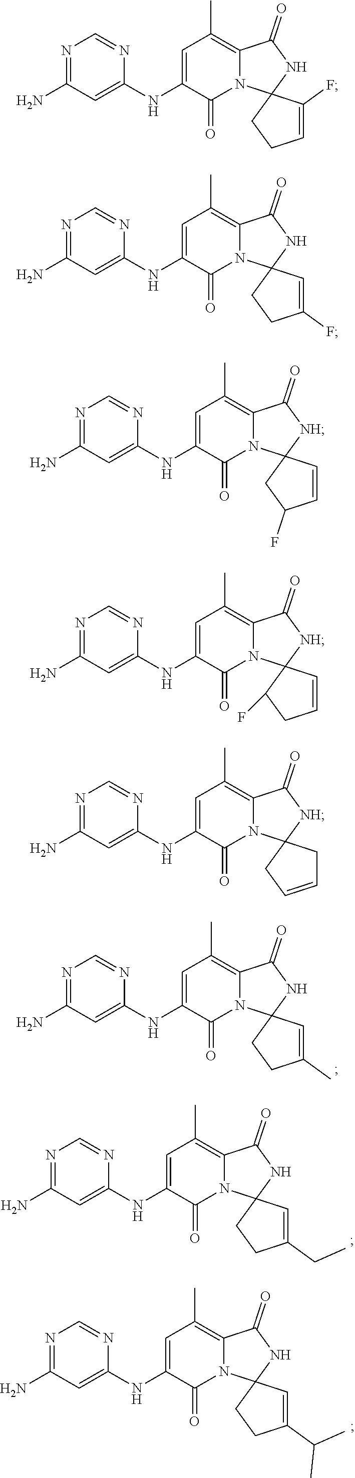 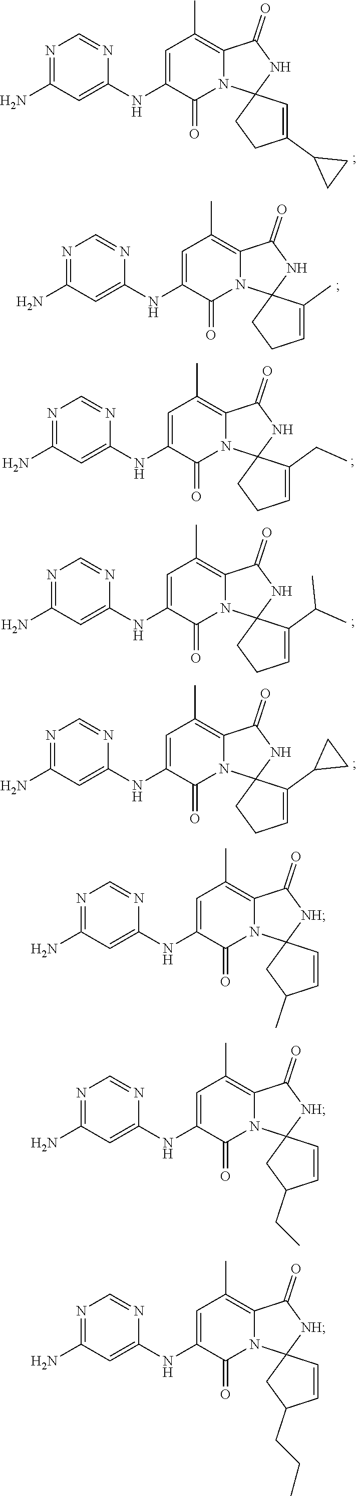 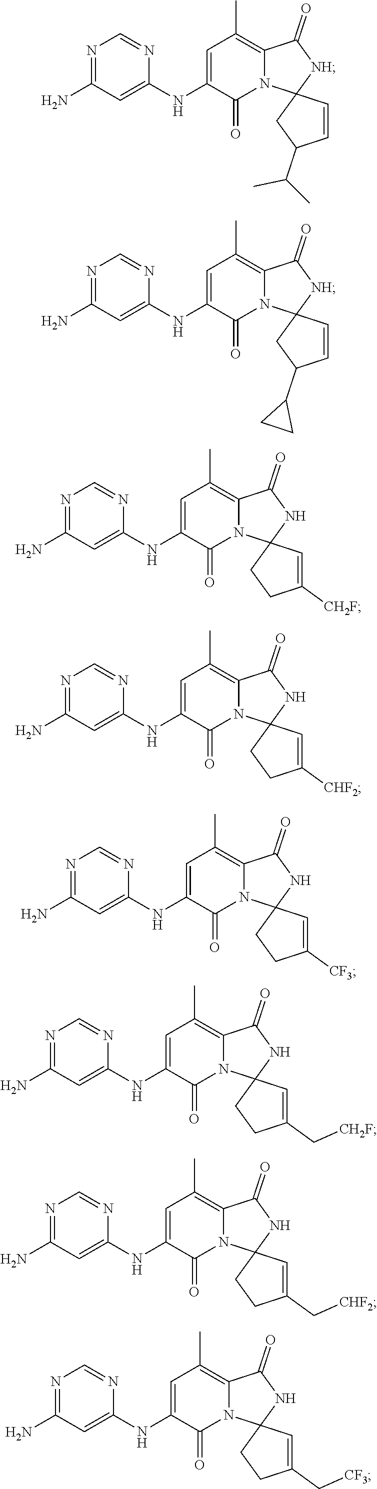 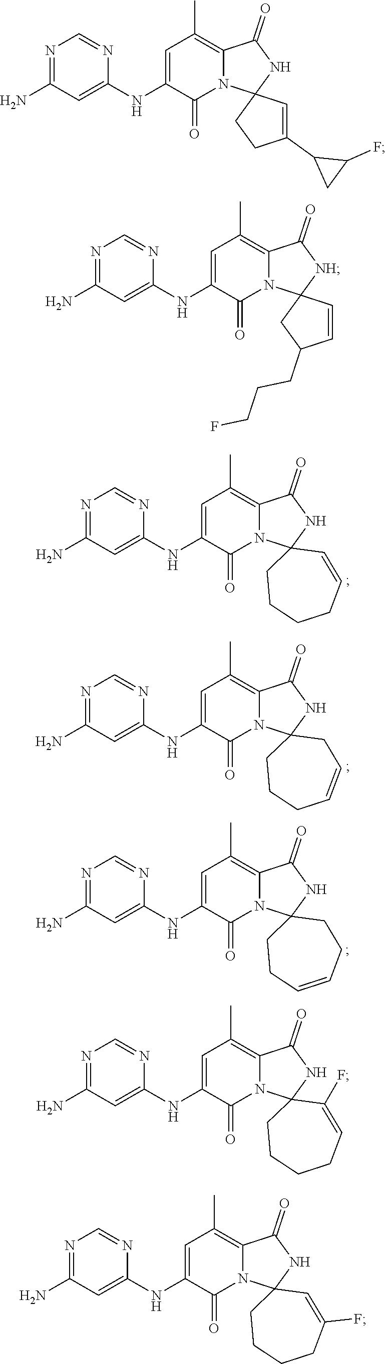 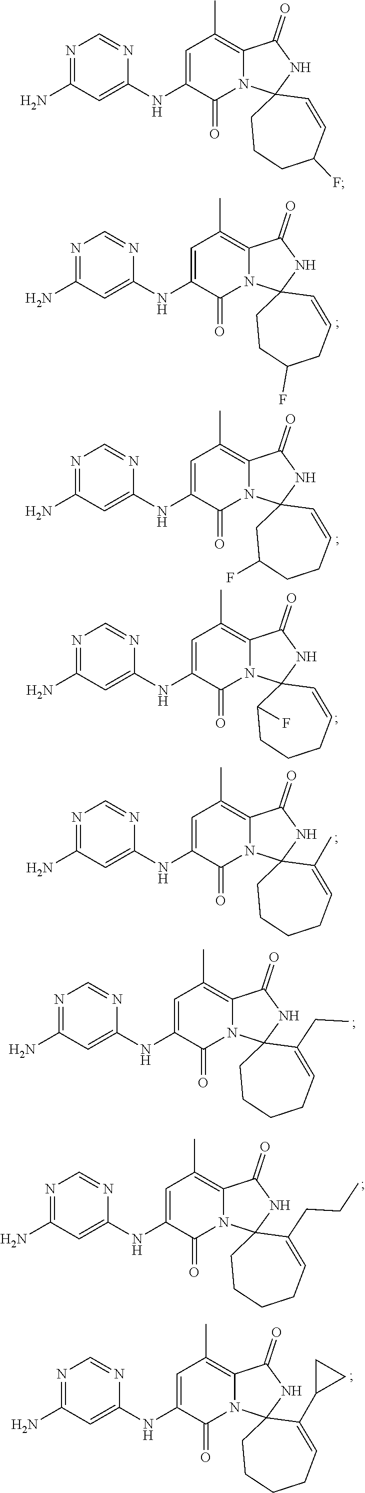 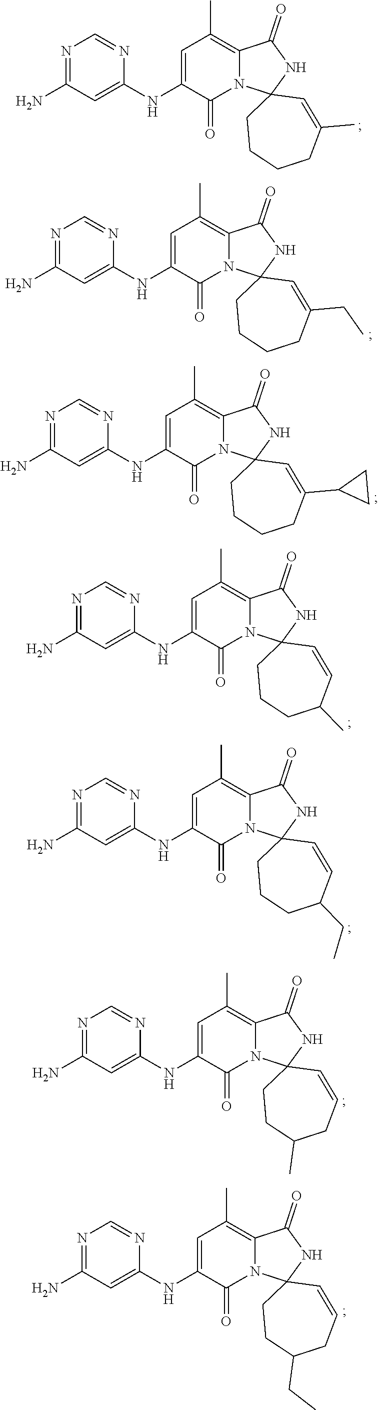 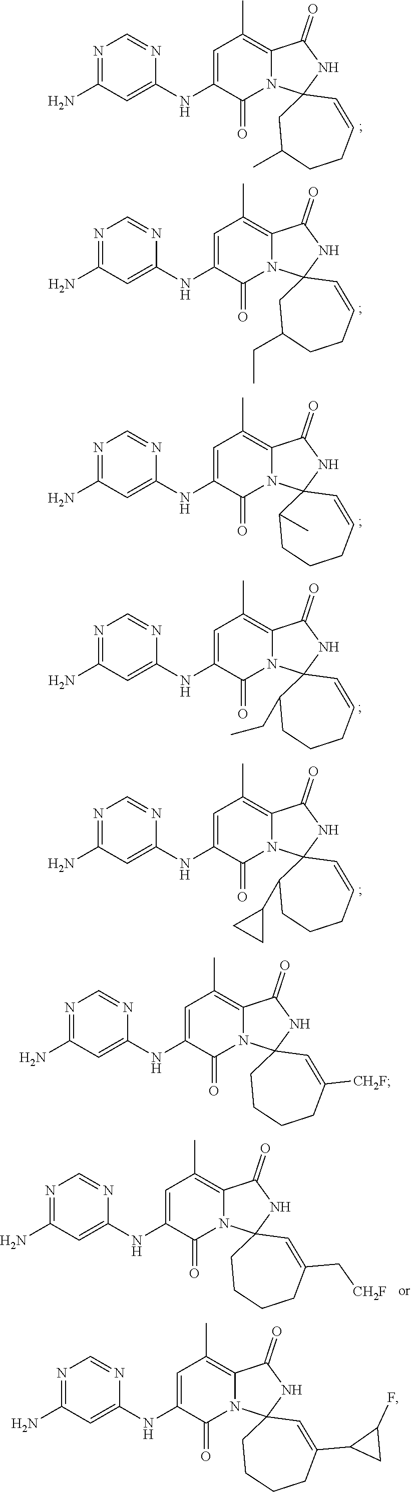
or a pharmaceutically acceptable salt, stereoisomer, tautomer, or prodrug thereof to a subject in need thereof.
